SEQUENCE LISTING (42 sequences)

SEQ ID NO: 1 - protein, 120 aa, Homo sapiens
EVTLKESGGGLVQPGGSRKLSCAASGFTFSTFGIHWVRQAPEKGLEWVAYISGASTTIYYADTVKGRFTISRDNPKNTLFLQMTSLRSEDTAMYYCARYGTSFPYGLDYWGQGTSVTVSS

SEQ ID NO: 2 - protein, 120 aa, Homo sapiens
EVTLVESGGGLVQPGGSRKLSCAASGFTFSTFGIHWVRQAPGKGLEWVAYISGASTTIYYADTVKGRFTISRDNSKNTLYLQMNSLRSEDTAMYYCARYGTSFPYGLDYWGQGTSVTVSS

SEQ ID NO: 3 - protein, 120 aa, Homo sapiens
EVTLVESGGGLVQPGGSLKLSCAASGFTFSTFGIHWVRQAPGKGLEWVAYISGASTTIYYADTVKGRFTISRDNSKNTLYLQMNSLRSEDTAMYYCARYGTSFPYGLDYWGQGTLVTVSS

SEQ ID NO: 4 - protein, 120 aa, Homo sapiens
EVQLVESGGGLVQPGGSLKLSCAASGFTFSTFGIHWVRQAPGKGLEWVAYISGASTTIYYADTVKGRFTISRDNSKNTLYLQMNSLRSEDTAMYYCARYGTSFPYGLDYWGQGTLVTVSS

SEQ ID NO: 5 - protein, 120 aa, Homo sapiens
EVQLVESGGGLVQPGGSLKLSCAASGFTFSTFGIHWVRQAPGKGLEWVAYISGASTTIYYADTVKGRFTISRDNSKNTLYLQMNSLRAEDTAVYYCARYGTSFPYGLDYWGQGTLVTVSS

SEQ ID NO: 6 - protein, 120 aa, Homo sapiens
EVQLVESGGGLVQPGGSLKLSCAASGFTFSTFGIHWVRQAPGKGLEWVAYISGASTTIYYADSVKGRFTISRDNSKNTLYLQMNSLRAEDTAVYYCARYGTSFPYGLDYWGQGTLVTVSS

SEQ ID NO: 7 - protein, 120 aa, Homo sapiens
EVQLVESGGGLVQPGGSLKLSCAASGFTFSTFGIHWVRQAPGKGLEWVASISGASTTIYYADSVKGRFTISRDNSKNTLYLQMNSLRAEDTAVYYCARYGTSFPYGLDYWGQGTLVTVSS

SEQ ID NO: 8 - protein, 111 aa, Homo sapiens
DIVMTQSPGSLAVSLGQRATISCKASQSVDYDGDSYMNWYQQKPGQPPKLLIYGASNLESGIPARFSGSGSGTDFTLNIHPVEEEDAASYYCQQSNEDPFTFGSGTKLEIK

SEQ ID NO: 9 - protein, 111 aa, Homo sapiens
DIVMTQSPGSLAVSLGERATINCKASQSVDYDGDSYMNWYQQKPGKAPKLLIYGASNLESGIPDRFSGSGSGTDFTLTISRLEEEDAASYYCQQSNEDPFTFGQGTKLEIK

SEQ ID NO: 10 - protein, 111 aa, Homo sapiens
DIVMTQSPDSLAVSLGERATINCKASQSVDYDGDSYMNWYQQKPGKAPKLLIYGASNLESGIPDRFSGSGSGTDFTLTISRLEPEDAASYYCQQSNEDPFTFGQGTKLEIK

SEQ ID NO: 11 - protein, 111 aa, Homo sapiens
DIVMTQSPDSLAVSLGERATINCKASQSVDYDGDSYMNWYQQKPGKAPKLLIYGASNLESGIPDRFSGSGSGTDFTLTISRLEPEDAAVYYCQQSNEDPFTFGQGTKLEIK

SEQ ID NO: 12 - protein, 111 aa, Homo sapiens
DIVMTQSPDSLAVSLGERATINCKSSQSVDYDGDSYMNWYQQKPGKAPKLLIYGASNLESGIPDRFSGSGSGTDFTLTISRLEPEDAASYYCQQSNEDPFTFGQGTKLEIK

SEQ ID NO: 13 - protein, 111 aa, Homo sapiens
DIVMTQSPDSLAVSLGERATINCKSSQSVDYDGDSYMNWYQQKPGKAPKLLIYGASNLESGIPDRFSGSGSGTDFTLTISRLEPEDAAVYYCQQSNEDPFTFGQGTKLEIK

SEQ ID NO: 14 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 107 aa, Homo sapiens [sequence omitted]

SEQ ID NO: 16 - protein, 5 aa, Homo sapiens
TFGIH

SEQ ID NO: 17 - protein, 17 aa, Homo sapiens
YISGASTTIYYADTVKG

SEQ ID NO: 18 - protein, 17 aa, Homo sapiens
YISGASTTIYYADSVKG

SEQ ID NO: 19 - protein, 17 aa, Homo sapiens
SISGASTTIYYADSVKG

SEQ ID NO: 20 - protein, 11 aa, Homo sapiens
YGTSFPYGLDY

SEQ ID NO: 21 - protein, 15 aa, Homo sapiens
KASQSVDYDGDSYMN

SEQ ID NO: 22 - protein, 15 aa, Homo sapiens
KSSQSVDYDGDSYMN

SEQ ID NO: 23 - protein, 7 aa, Homo sapiens
GASNLES

SEQ ID NO: 24 - protein, 9 aa, Homo sapiens
QQSNEDPFT

SEQ ID NO: 25 - DNA, 360 bp, Homo sapiens
  source 1..360: mol_type = other DNA
gaagtgaccctgaaagagtctggcggaggactggttcagcctggcggatctagaaagctgtcttgtgccgcctccggcttcaccttctctacctttggcatccactgggtccgacaggcccctgagaaaggattggagtgggtcgcctacatctccggcgcttccaccaccatctactacgccgataccgtgaagggcagattcaccatcagccgggacaaccccaagaacaccctgtttctgcagatgaccagcctgcggagcgaggacaccgccatgtactactgtgccagatacggcaccagctttccctacggcctggattattggggccagggaacctccgttacagtctcctca

SEQ ID NO: 26 - DNA, 360 bp, Homo sapiens
  source 1..360: mol_type = other DNA
gaagtgaccctggttgaatctggcggaggactggttcagcctggcggctctagaaagctgtcttgtgccgcctctggcttcaccttctccacctttggcatccactgggtccgacaggcccctggcaaaggattggagtgggtcgcctatatctccggcgcctccaccaccatctactacgccgataccgtgaagggcagattcaccatcagccgggacaactccaagaacaccctgtacctgcagatgaactccctgcggagcgaggacaccgccatgtactactgtgccagatacggcaccagctttccctacggcctggattattggggccagggaacctccgttacagtctcctca

SEQ ID NO: 27 - DNA, 360 bp, Homo sapiens
  source 1..360: mol_type = other DNA
gaagtgaccctggttgaatctggcggaggactggttcagcctggcggatctctgaagctgtcttgtgccgcctctggcttcaccttctccacctttggcatccactgggtccgacaggcccctggcaaaggattggagtgggtcgcctatatctccggcgcctccaccaccatctactacgccgataccgtgaagggcagattcaccatcagccgggacaactccaagaacaccctgtacctgcagatgaactccctgcggagcgaggacaccgccatgtactactgtgccagatacggcaccagctttccctacggcctggattattggggccagggaaccctggttacagtctcctca

SEQ ID NO: 28 - DNA, 360 bp, Homo sapiens
  source 1..360: mol_type = other DNA
gaagtgcagctggttgaatctggcggaggactggttcagcctggcggatctctgaagctgtcttgtgccgcctctggcttcaccttctccacctttggcatccactgggtccgacaggcccctggcaaaggattggagtgggtcgcctatatctccggcgcctccaccaccatctactacgccgataccgtgaagggcagattcaccatcagccgggacaactccaagaacaccctgtacctgcagatgaactccctgcggagcgaggacaccgccatgtactactgtgccagatacggcaccagctttccctacggcctggattattggggccagggaaccctggttacagtctcctca

SEQ ID NO: 29 - DNA, 360 bp, Homo sapiens
  source 1..360: mol_type = other DNA
gaagtgcagctggttgaatctggcggaggactggttcagcctggcggatctctgaagctgtcttgtgccgcctctggcttcaccttctccacctttggcatccactgggtccgacaggcccctggcaaaggattggagtgggtcgcctatatctccggcgcctccaccaccatctactacgccgataccgtgaagggcagattcaccatcagccgggacaactccaagaacaccctgtacctgcagatgaactccctgagagccgaggacaccgccgtgtactactgtgccagatacggcaccagctttccctacggcctggattattggggccagggaaccctggttacagtctcctca

SEQ ID NO: 30 - DNA, 360 bp, Homo sapiens
  source 1..360: mol_type = other DNA
gaagtgcagctggttgaatctggcggaggactggttcagcctggcggatctctgaagctgtcttgtgccgcctctggcttcaccttctccacctttggcatccactgggtccgacaggcccctggcaaaggattggagtgggtcgcctatatctccggcgcctccaccaccatctactacgccgattctgtgaagggcagattcaccatcagccgggacaactccaagaacaccctgtacctgcagatgaactccctgagagccgaggacaccgccgtgtactactgtgccagatacggcaccagctttccctacggcctggattattggggccagggaaccctggttacagtctcctca

SEQ ID NO: 31 - DNA, 360 bp, Homo sapiens
  source 1..360: mol_type = other DNA
gaagtgcagctggttgaatctggcggaggactggttcagcctggcggatctctgaagctgtcttgtgccgcctctggcttcaccttctccacctttggcatccactgggtccgacaggcccctggcaaaggattggaatgggtcgcctctatctccggcgcctccaccaccatctactacgccgattctgtgaagggcagattcaccatcagccgggacaactccaagaacaccctgtacctgcagatgaactccctgagagccgaggacaccgccgtgtactactgtgccagatacggcaccagctttccctacggcctggattattggggccagggaaccctggttacagtctcctca

SEQ ID NO: 32 - DNA, 333 bp, Homo sapiens
  source 1..333: mol_type = other DNA
gacatcgtgatgacacagtctccaggcagcctggctgtgtccttgggacagagagctaccatctcctgcaaggcctctcagtccgtggactacgacggcgactcctacatgaactggtatcagcagaagcccggccagcctcctaagctgttgatctacggcgcctccaacctggaaagcggcatccctgctagattctccggctctggctctggcaccgacttcaccctgaacatccatcctgtggaagaagaggacgccgcctcctactactgccagcagtctaacgaggaccccttcaccttcggctccggcacaaaattggagatcaaa

SEQ ID NO: 33 - DNA, 333 bp, Homo sapiens
  source 1..333: mol_type = other DNA
gacatcgtgatgacacagtctccaggcagcctggctgtgtctctgggagagagagccaccatcaactgcaaggcctctcagtccgtggactacgacggcgactcctacatgaactggtatcagcagaagcccggcaaggcccctaagctgttgatctacggcgcctccaacctggaaagcggcatccctgatagattctccggctctggctctggcaccgacttcaccctgaccatctctcggctggaagaggaagatgccgcctcctactactgccagcagtccaacgaggaccccttcacctttggccagggcacaaagttggagatcaaa

SEQ ID NO: 34 - DNA, 333 bp, Homo sapiens
  source 1..333: mol_type = other DNA
gacatcgtgatgacccagtctccagacagcctggctgtgtctctgggcgagagagccaccatcaactgcaaggcctctcagtccgtggactacgacggcgactcctacatgaactggtatcagcagaagcccggcaaggcccctaagctgttgatctacggcgcctccaacctggaaagcggcatccctgatagattctccggctctggctctggcaccgacttcaccctgaccatctccagactggaacctgaggacgccgcctcctactactgccagcagtctaacgaggaccccttcacctttggccagggcacaaagttggagatcaaa

SEQ ID NO: 35 - DNA, 333 bp, Homo sapiens
  source 1..333: mol_type = other DNA
gacatcgtgatgacccagtctccagacagcctggctgtgtctctgggcgagagagccaccatcaactgcaaggcctctcagtccgtggactacgacggcgactcctacatgaactggtatcagcagaagcccggcaaggcccctaagctgttgatctacggcgcctccaacctggaaagcggcatccctgatagattctccggctctggctctggcaccgacttcaccctgaccatctccagactggaacctgaggatgccgccgtgtactactgccagcagtctaacgaggaccccttcacctttggccagggcacaaagttggagatcaaa

SEQ ID NO: 36 - DNA, 333 bp, Homo sapiens
  source 1..333: mol_type = other DNA
gacatcgtgatgacccagtctccagacagcctggctgtgtctctgggcgagagagccaccatcaactgcaagtcctctcagtccgtggactacgacggcgactcctacatgaactggtatcagcagaagcccggcaaggcccctaagctgttgatctacggcgcctccaacctggaaagcggcatccctgatagattctccggctctggctctggcaccgacttcaccctgaccatctccagactggaacctgaggacgccgcctcctactactgccagcagtctaacgaggaccccttcacctttggccagggcacaaagttggagatcaaa

SEQ ID NO: 37 - DNA, 333 bp, Homo sapiens
  source 1..333: mol_type = other DNA
gacatcgtgatgacccagtctccagacagcctggctgtgtctctgggcgagagagccaccatcaactgcaagtcctctcagtccgtggactacgacggcgactcctacatgaactggtatcagcagaagcccggcaaggcccctaagctgttgatctacggcgcctccaacctggaaagcggcatccctgatagattctccggctctggctctggcaccgacttcaccctgaccatctccagactggaacctgaggatgccgccgtgtactactgccagcagtctaacgaggaccccttcacctttggccagggcacaaagttggagatcaaa

SEQ ID NO: 38 - protein, 450 aa, Homo sapiens
EVQLVESGGGLVQPGGSLKLSCAASGFTFSTFGIHWVRQAPGKGLEWVAYISGASTTIYYADTVKGRFTISRDNSKNTLYLQMNSLRAEDTAVYYCARYGTSFPYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 450 aa, Homo sapiens
EVQLVESGGGLVQPGGSLKLSCAASGFTFSTFGIHWVRQAPGKGLEWVAYISGASTTIYYADSVKGRFTISRDNSKNTLYLQMNSLRAEDTAVYYCARYGTSFPYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 218 aa, Homo sapiens
DIVMTQSPDSLAVSLGERATINCKASQSVDYDGDSYMNWYQQKPGKAPKLLIYGASNLESGIPDRFSGSGSGTDFTLTISRLEPEDAAVYYCQQSNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 1037 aa, Homo sapiens
MRGSGPRGAGRRRPPSGGGDTPITPASLAGCYSAPRRAPLWTCLLLCAALRTLLASPSNEVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKKCPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQAAPSPVTNVKKGKIAKNSISLSWQEPDRPNGIILEYEIKYFEKDQETSYTIIKSKETTITAEGLKPASVYVFQIRARTAAGYGVFSRRFEFETTPVFAASSDQSQIPVIAVSVTVGVILLAVVIGVLLSGSCCECGCGRASSLCAVAHPSLIWRCGYSKAKQDPEEEKMHFHNGHIKLPGVRTYIDPHTYEDPNQAVHEFAKEIEASCITIERVIGAGEFGEVCSGRLKLPGKRELPVAIKTLKVGYTEKQRRDFLGEASIMGQFDHPNIIHLEGVVTKSKPVMIVTEYMENGSLDTFLKKNDGQFTVIQLVGMLRGISAGMKYLSDMGYVHRDLAARNILINSNLVCKVSDFGLSRVLEDDPEAAYTTRGGKIPIRWTAPEAIAFRKFTSASDVWSYGIVMWEVVSYGERPYWEMTNQDVIKAVEEGYRLPSPMDCPAALYQLMLDCWQKERNSRPKFDEIVNMLDKLIRNPSSLKTLVNASCRVSNLLAEHSPLGSGAYRSVGEWLEAIKMGRYTEIFMENGYSSMDAVAQVTLEDLRRLGVTLVGHQKKIMNSLQEMKVQLVNGMVPL

SEQ ID NO: 42 - protein, 10 aa, Homo sapiens
GFTFSTFGIH

(19) United States
(12) Patent Application Publication
(10) Pub. No.: US 2025/0302984 A1
(43) Pub. Date: Oct. 2, 2025

(54) Antibody-Drug Conjugates Against EPH Receptor A5 (Epha5) for Treatment of Carcinoma

(71) Applicant: MBrace Therapeutics, Inc., San Diego, CA (US)

(72) Inventors: Fernanda I. STAQUICINI, Jersey City, NJ (US); Steve Alley, Seattle, WA (US)

(73) Assignee: MBrace Therapeutics, Inc., San Diego, CA (US)

(21) Appl. No.: 19/094,543

(22) Filed: Mar. 28, 2025

Related U.S. Application Data
(60) Provisional application No. 63/571,291, filed on Mar. 28, 2024.
(60) Provisional application No. 63/736,175, filed on Dec. 19, 2024.

(51) Int. Cl. A61K 47/68; A61P 35/00

(52) U.S. Cl. CPC A61K 47/6889; A61K 47/68031; A61K 47/6849; A61P 35/00

(57) ABSTRACT

The present invention relates to immunoconjugates specific for human EPH receptor A5 (EphA5), methods of treatment of cancers (e.g., carcinomas including squamous cell carcinoma, and colorectal cancer) using said immunoconjugates, and related pharmaceutical compositions of said immunoconjugates.

### RELATED APPLICATIONS

This application claims priority to U.S. Provisional Patent Application Ser. No. 63/571,291 filed on Mar. 28, 2024, and U.S. Provisional Patent Application Ser. No. 63/736,175 filed on Dec. 19, 2024, each of which is hereby incorporated by reference into this application in its entirety.

### INCORPORATION OF SEQUENCE LISTING

A computer readable form of the Sequence Listing is filed with this application by electronic submission and is incorporated into this application by reference in its entirety. The Sequence Listing is contained in the file created on Mar. 28, 2025, having the file name MBRACE006.xml, and is 45,639 bytes in size (as measured in the MS-Windows® operating system).

### FIELD OF THE DISCLOSURE

This disclosure relates to antibodies, binding polypeptides, and immunoconjugates including antibody-drug conjugates (ADCs) having binding activity for human EPH receptor A5 (EphA5), and compositions comprising and methods for using as well as making the same.

### BACKGROUND

EphA5 is part of the Eph receptor family, a subgroup of receptor tyrosine kinases (RTKs) that are involved in various biological processes, including cell-cell communication, neural development, and the regulation of cell migration and positioning. EphA5 is expressed in the nervous system where it plays a role in axon guidance and the development of neural circuits, but it is also involved in various signaling pathways related to cell growth, survival, and differentiation. EphA5 and its signaling pathways are of interest for therapeutic research, particularly in cancer, where EphA5 has been shown to be expressed on some cancers (Staquicini F I, et al. Receptor tyrosine kinase EphA5 is a functional molecular target in human lung cancer. J Biol Chem. 2015; 290(12):7345-7359; Chen X, et al. EphA5 protein, a potential marker for distinguishing histological grade and prognosis in ovarian serous carcinoma. J Ovarian Res. 2016; 9(83):1-7; Giaginis C, et al. Clinical significance of ephrin (eph)-A1, -A2, -A4, -A5 and -A7 receptors in pancreatic ductal adenocarcinoma. Pathol Oncol Res. 2010; 16(2):267-276). Antibodies against EphA5 have been shown to sensitize lung cancer cells to radiation (Staquicini F I, et al. Receptor tyrosine kinase EphA5 is a functional molecular target in human lung cancer. J Biol Chem. 2015; 290(12):7345-7359).

Antibody-drug conjugates (ADC) have been proven to be a biologic that can deliver drug to targeted cells with high efficiency and minimized off-target effect. The receptor tyrosine kinase EphA5 is a surface molecule expressed in many types of human cancer cell lines and biopsies.

Previous efforts to target EphA5 have been ineffective. Small molecule EphA5 modulators are not targeted to cancerous cells and therefore are generally unacceptably toxic to healthy cells and tissues. On the other hand, mAb-targeted EphA5 modulators have been exceedingly difficult to produce in view of toxicities to the expressing cells. Effective therapies against cancer expressing EphA5 therefore remain urgently needed for numerous cancer indications, including in particular castration resistant prostate cancer.

### SUMMARY OF THE INVENTION

Cancer therapies targeting tumor-specific targets like EphA5, like those disclosed herein, address the needs identified above.

EphA5 has been found to be overexpressed in some cancer types, including but not limited to lung, prostate, and breast cancers. The present invention relates in certain aspects to antibodies, binding polypeptides, and immunoconjugates specific for human EPH receptor A5 (EphA5), and methods of treatment using these molecules and/or fragments thereof, including antibody-drug conjugates comprising these molecules and/or fragments thereof.

In certain aspects, this disclosure described antibody-drug conjugates, or ADCs (discussed in more detail below), comprising one or more of the antibodies or antibody fragments described herein.

In certain aspects, the invention provides an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising a heavy chain variable region comprising an amino acid sequence having at least about 85%, 90%, 91%, 92%, 93%, 94%, 95%, 96%, 97%, 98%, or 99% sequence identity to SEQ ID NO: 1.

In certain embodiments of the above aspects or any other aspect or embodiment disclosed herein, the antibody or antigen-binding fragment comprises a light chain comprising a light chain variable region comprising an amino acid sequence having at least about 85%, 90%, 91%, 92%, 93%, 94%, 95%, 96%, 97%, 98%, or 99% sequence identity to SEQ ID NO: 8.

In certain embodiments of the above aspects or any other aspect or embodiment disclosed herein, the heavy chain variable region comprises three heavy chain complementarity-determining regions (HCDRs), wherein HCDR1 comprises the amino acid sequence set forth in SEQ ID NO: 16, HCDR2 comprises an amino acid sequence selected from the group consisting of SEQ ID NOs: 17-19, and HCDR3 comprises the amino acid sequence set forth in SEQ ID NO: 20.

In certain embodiments of the above aspects or any other aspect or embodiment disclosed herein, the light chain variable region comprises three light chain complementarity-determining regions (LCDRs), wherein LCDR1 comprises an amino acid sequence selected from the group consisting of SEQ ID NOs: 21 and 22, LCDR2 comprises the amino acid sequence set forth in SEQ ID NO: 23, and LCDR3 comprises the amino acid sequence set forth in SEQ ID NO: 24.

In certain embodiments, the invention provides the antibody or antigen-binding fragment described above or herein, and/or aspects or features of said antibody or antigen-binding fragment.

In certain embodiments or aspects thereof described above or herein, the heavy chain variable region comprises a HCDR1 comprising the amino acid sequence set forth in SEQ ID NO: 16, a HCDR2 comprising the amino acid sequence set forth in SEQ ID NO: 17, and a HCDR3 comprising the amino acid sequence set forth in SEQ ID NO: 20; and the light chain variable region comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain variable region comprises a HCDR1 comprising the amino acid sequence set forth in SEQ ID NO: 16, a HCDR2 comprising the amino acid sequence set forth in SEQ ID NO: 18, and a HCDR3 comprising the amino acid sequence set forth in SEQ ID NO: 20; and the light chain variable region comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain variable region comprises a HCDR1 comprising the amino acid sequence set forth in SEQ ID NO: 16, a HCDR2 comprising the amino acid sequence set forth in SEQ ID NO: 19, and a HCDR3 comprising the amino acid sequence set forth in SEQ ID NO: 20; and the light chain variable region comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain variable region comprises a HCDR1 comprising the amino acid sequence set forth in SEQ ID NO: 16, a HCDR2 comprising the amino acid sequence set forth in SEQ ID NO: 17, and a HCDR3 comprising the amino acid sequence set forth in SEQ ID NO: 20; and the light chain variable region comprises a LCDR1 comprising the amino acid sequence set forth in SEQ ID NO: 22, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain variable region comprises a HCDR1 comprising the amino acid sequence set forth in SEQ ID NO: 16, a HCDR2 comprising the amino acid sequence set forth in SEQ ID NO: 18, and a HCDR3 comprising the amino acid sequence set forth in SEQ ID NO: 20; and the light chain variable region comprises a LCDR1 comprising the amino acid sequence set forth in SEQ ID NO: 22, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain variable region comprises a HCDR1 comprising the amino acid sequence set forth in SEQ ID NO: 16, a HCDR2 comprising the amino acid sequence set forth in SEQ ID NO: 19, and a HCDR3 comprising the amino acid sequence set forth in SEQ ID NO: 20; and the light chain variable region comprises a LCDR1 comprising the amino acid sequence set forth in SEQ ID NO: 22, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the amino acid sequence of the heavy chain variable region has at least about 85%, 90%, 91%, 92%, 93%, 94%, 95%, 96%, 97%, 98%, or 99% sequence identity to an amino acid sequence selected from the group consisting of SEQ ID NOs: 2-7.

In certain embodiments of the above aspects or any other aspect or embodiment disclosed herein, the heavy chain variable region comprises an amino acid sequence selected from the group consisting of SEQ ID NOs: 2-7.

In certain embodiments of the above aspects or any other aspect or embodiment disclosed herein, the heavy chain variable region consists of an amino acid sequence selected from the group consisting of SEQ ID NOs: 2-7.

In certain embodiments of the above aspects or any other aspect or embodiment disclosed herein, the heavy chain variable region is encoded by a nucleic acid sequence selected from the group consisting of SEQ ID NOs: 25-31.

In certain embodiments of the above aspects or any other aspect or embodiment disclosed herein, the amino acid sequence of the light chain variable region has at least about 85%, 90%, 95%, 96%, 97%, 98%, or 99% sequence identity to an amino acid sequence selected from the group consisting of SEQ ID NOs: 9-13.

In certain embodiments of the above aspects or any other aspect or embodiment disclosed herein, the light chain variable region comprises an amino acid sequence selected from the group consisting of SEQ ID NOs: 9-13.

In certain embodiments of the above aspects or any other aspect or embodiment disclosed herein, the light chain variable region consists of an amino acid sequence selected from the group consisting of SEQ ID NOs: 9-13.

In certain embodiments of the above aspects or any other aspect or embodiment disclosed herein, the light chain variable region is encoded by a nucleic acid sequence selected from the group consisting of SEQ ID NOs: 32-37.

In certain embodiments of the above aspects or any other aspect or embodiment disclosed herein, the heavy chain variable region is set forth in SEQ ID NO: 2 and the light chain variable region is set forth in SEQ ID NO: 9.

In certain embodiments of the above aspects or any other aspect or embodiment disclosed herein, the heavy chain variable region is set forth in SEQ ID NO: 2 and the light chain variable region is set forth in SEQ ID NO: 10.

In certain embodiments of the above aspects or any other aspect or embodiment disclosed herein, the heavy chain variable region is set forth in SEQ ID NO: 2 and the light chain variable region is set forth in SEQ ID NO: 11.

In certain embodiments of the above aspects or any other aspect or embodiment disclosed herein, the heavy chain variable region is set forth in SEQ ID NO: 2 and the light chain variable region is set forth in SEQ ID NO: 12.

In certain embodiments of the above aspects or any other aspect or embodiment disclosed herein, the heavy chain variable region is set forth in SEQ ID NO: 2 and the light chain variable region is set forth in SEQ ID NO: 13.

In certain embodiments of the above aspects or any other aspect or embodiment disclosed herein, the heavy chain variable region is set forth in SEQ ID NO: 3 and the light chain variable region is set forth in SEQ ID NO: 9.

In certain embodiments of the above aspects or any other aspect or embodiment disclosed herein, the heavy chain variable region is set forth in SEQ ID NO: 3 and the light chain variable region is set forth in SEQ ID NO: 10.

In certain embodiments of the above aspects or any other aspect or embodiment disclosed herein, the heavy chain variable region is set forth in SEQ ID NO: 3 and the light chain variable region is set forth in SEQ ID NO: 11.

In certain embodiments of the above aspects or any other aspect or embodiment disclosed herein, the heavy chain variable region is set forth in SEQ ID NO: 3 and the light chain variable region is set forth in SEQ ID NO: 12.

In certain embodiments of the above aspects or any other aspect or embodiment disclosed herein, the heavy chain variable region is set forth in SEQ ID NO: 3 and the light chain variable region is set forth in SEQ ID NO: 13.

In certain embodiments of the above aspects or any other aspect or embodiment disclosed herein, the heavy chain variable region is set forth in SEQ ID NO: 4 and the light chain variable region is set forth in SEQ ID NO: 9.

In certain embodiments of the above aspects or any other aspect or embodiment disclosed herein, the heavy chain variable region is set forth in SEQ ID NO: 4 and the light chain variable region is set forth in SEQ ID NO: 10.

In certain embodiments of the above aspects or any other aspect or embodiment disclosed herein, the heavy chain variable region is set forth in SEQ ID NO: 4 and the light chain variable region is set forth in SEQ ID NO: 11.

In certain embodiments of the above aspects or any other aspect or embodiment disclosed herein, the heavy chain variable region is set forth in SEQ ID NO: 4 and the light chain variable region is set forth in SEQ ID NO: 12.

In certain embodiments of the above aspects or any other aspect or embodiment disclosed herein, the heavy chain variable region is set forth in SEQ ID NO: 4 and the light chain variable region is set forth in SEQ ID NO: 13.

In certain embodiments of the above aspects or any other aspect or embodiment disclosed herein, the heavy chain variable region is set forth in SEQ ID NO: 5 and the light chain variable region is set forth in SEQ ID NO: 9.

In certain embodiments of the above aspects or any other aspect or embodiment disclosed herein, the heavy chain variable region is set forth in SEQ ID NO: 5 and the light chain variable region is set forth in SEQ ID NO: 10.

In certain embodiments of the above aspects or any other aspect or embodiment disclosed herein, the heavy chain variable region is set forth in SEQ ID NO: 5 and the light chain variable region is set forth in SEQ ID NO: 11.

In certain embodiments of the above aspects or any other aspect or embodiment disclosed herein, the heavy chain variable region is set forth in SEQ ID NO: 5 and the light chain variable region is set forth in SEQ ID NO: 12.

In certain embodiments of the above aspects or any other aspect or embodiment disclosed herein, the heavy chain variable region is set forth in SEQ ID NO: 5 and the light chain variable region is set forth in SEQ ID NO: 13.

In certain embodiments of the above aspects or any other aspect or embodiment disclosed herein, the heavy chain variable region is set forth in SEQ ID NO: 6 and the light chain variable region is set forth in SEQ ID NO: 9.

In certain embodiments of the above aspects or any other aspect or embodiment disclosed herein, the heavy chain variable region is set forth in SEQ ID NO: 6 and the light chain variable region is set forth in SEQ ID NO: 10.

In certain embodiments of the above aspects or any other aspect or embodiment disclosed herein, the heavy chain variable region is set forth in SEQ ID NO: 6 and the light chain variable region is set forth in SEQ ID NO: 11.

In certain embodiments of the above aspects or any other aspect or embodiment disclosed herein, the heavy chain variable region is set forth in SEQ ID NO: 6 and the light chain variable region is set forth in SEQ ID NO: 12.

In certain embodiments of the above aspects or any other aspect or embodiment disclosed herein, the heavy chain variable region is set forth in SEQ ID NO: 6 and the light chain variable region is set forth in SEQ ID NO: 13.

In certain embodiments of the above aspects or any other aspect or embodiment disclosed herein, the heavy chain variable region is set forth in SEQ ID NO: 7 and the light chain variable region is set forth in SEQ ID NO: 9.

In certain embodiments of the above aspects or any other aspect or embodiment disclosed herein, the heavy chain variable region is set forth in SEQ ID NO: 7 and the light chain variable region is set forth in SEQ ID NO: 10.

In certain embodiments of the above aspects or any other aspect or embodiment disclosed herein, the heavy chain variable region is set forth in SEQ ID NO: 7 and the light chain variable region is set forth in SEQ ID NO: 11.

In certain embodiments of the above aspects or any other aspect or embodiment disclosed herein, the heavy chain variable region is set forth in SEQ ID NO: 7 and the light chain variable region is set forth in SEQ ID NO: 12.

In certain embodiments of the above aspects or any other aspect or embodiment disclosed herein, the heavy chain variable region is set forth in SEQ ID NO: 7 and the light chain variable region is set forth in SEQ ID NO: 13.

In certain embodiments of the above aspects or any other aspect or embodiment disclosed herein, the heavy chain variable region comprises a sequence that has at least 90% sequence identity to the sequence set forth in SEQ ID NO: 2 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 2 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 2 and the light chain variable region comprises a sequence that has at least 90% sequence identity to the sequence set forth in SEQ ID NO: 11.

In certain embodiments of the above aspects or any other aspect or embodiment disclosed herein, the heavy chain variable region comprises a sequence that has at least 90% sequence identity to the sequence set forth in SEQ ID NO: 2 and the light chain variable region comprises a sequence that has at least 90% sequence identity to the sequence set forth in SEQ ID NO: 12.

In certain embodiments of the above aspects or any other aspect or embodiment disclosed herein, the heavy chain variable region comprises a sequence that has at least 90% sequence identity to the sequence set forth in SEQ ID NO: 2 and the light chain variable region comprises a sequence that has at least 90% sequence identity to the sequence set forth in SEQ ID NO: 13.

In certain embodiments of the above aspects or any other aspect or embodiment disclosed herein, the heavy chain variable region comprises a sequence that has at least 90% sequence identity to the sequence set forth in SEQ ID NO: 3 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 3 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 3 and the light chain variable region comprises a sequence that has at least 90% sequence identity to the sequence set forth in SEQ ID NO: 11.

In certain embodiments of the above aspects or any other aspect or embodiment disclosed herein, the heavy chain variable region comprises a sequence that has at least 90% sequence identity to the sequence set forth in SEQ ID NO: 3 and the light chain variable region comprises a sequence that has at least 90% sequence identity to the sequence set forth in SEQ ID NO: 12.

In certain embodiments of the above aspects or any other aspect or embodiment disclosed herein, the heavy chain variable region comprises a sequence that has at least 90% sequence identity to the sequence set forth in SEQ ID NO: 3 and the light chain variable region comprises a sequence that has at least 90% sequence identity to the sequence forth in SEQ ID NO: 13.

In certain embodiments of the above aspects or any other aspect or embodiment disclosed herein, the heavy chain variable region comprises a sequence that has at least 90% sequence identity to the sequence set forth in SEQ ID NO: 4 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 4 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 4 and the light chain variable region comprises a sequence that has at least 90% sequence identity to the sequence set forth in SEQ ID NO: 11.

In certain embodiments of the above aspects or any other aspect or embodiment disclosed herein, the heavy chain variable region comprises a sequence that has at least 90% sequence identity to the sequence set forth in SEQ ID NO: 4 and the light chain variable region comprises a sequence that has at least 90% sequence identity to the sequence set forth in SEQ ID NO: 12.

In certain embodiments of the above aspects or any other aspect or embodiment disclosed herein, the heavy chain variable region comprises a sequence that has at least 90% sequence identity to the sequence set forth in SEQ ID NO: 4 and the light chain variable region comprises a sequence that has at least 90% sequence identity to the sequence set forth in SEQ ID NO: 13.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 11.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 12.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 13.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 11.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 12.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 13.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 11.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 12.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 13.

In certain aspects, the invention provides an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising a heavy chain variable region comprising a heavy chain complementarity-determining region 1 (HCDR1) comprising the sequence set forth in SEQ ID NO: 16, a HCDR2 comprising the sequence set forth in SEQ ID NO: 18, a HCDR3 set forth in SEQ ID NO: 20.

In certain embodiments of the above aspects or any other aspect or embodiment disclosed herein, the antibody or antigen-binding fragment comprises a light chain comprising a light chain variable region comprising a light chain complementarity determining region 1 (LCDR1) comprising the sequence set forth in SEQ ID NO: 21, a LCDR2 comprising the sequence set forth in SEQ ID NO: 23, and an LCDR3 comprising the sequence set forth in SEQ ID NO: 24.

In certain aspects, the invention provides an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising a heavy chain variable region comprising a heavy chain complementarity-determining region 1 (HCDR1) comprising the sequence set forth in SEQ ID NO: 16, a HCDR2 comprising the sequence set forth in SEQ ID NO: 19, a HCDR3 set forth in SEQ ID NO: 20.

In certain embodiments of the above aspects or any other aspect or embodiment disclosed herein, the antibody or antigen-binding fragment comprises a light chain comprising a light chain variable region comprising a light chain complementarity determining region 1 (LCDR1) comprising the sequence set forth in SEQ ID NO: 21, an LCDR2 comprising the sequence set forth in SEQ ID NO: 23, and an LCDR3 comprising the sequence set forth in SEQ ID NO: 24.

In certain aspects, the invention provides an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising a heavy chain variable region comprising a heavy chain complementarity-determining region 1 (HCDR1) comprising the sequence set forth in SEQ ID NO: 16, a HCDR2 comprising the sequence set forth in SEQ ID NO: 17, a HCDR3 set forth in SEQ ID NO: 20.

In certain embodiments of the above aspects or any other aspect or embodiment disclosed herein, the antibody or antigen-binding fragment comprises a light chain comprising a light chain variable region comprising a complementarity determining region 1 (LCDR1) comprising the sequence set forth in SEQ ID NO: 22, an LCDR2 comprising the sequence set forth in SEQ ID NO: 23, and an LCDR3 comprising the sequence set forth in SEQ ID NO: 24.

In certain aspects, the invention provides an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising a heavy chain variable region comprising a heavy chain complementarity-determining region 1 (HCDR1) comprising the sequence set forth in SEQ ID NO: 16, a HCDR2 comprising the sequence set forth in SEQ ID NO: 18, a HCDR3 set forth in SEQ ID NO: 20.

In certain embodiments of the above aspects or any other aspect or embodiment disclosed herein, the antibody or antigen-binding fragment comprises a light chain comprising a light chain variable region comprising a light chain complementarity determining region 1 (LCDR1) comprising the sequence set forth in SEQ ID NO: 22, LCDR2 comprising the sequence set forth in SEQ ID NO: 23, and an LCDR3 comprising the sequence set forth in SEQ ID NO: 24.

In certain aspects, the invention provides an antibody or antigen-binding fragment thereof.

In certain embodiments of the above aspects or any other aspect or embodiment disclosed herein, the antibody or antigen-binding fragment comprises a heavy chain comprising a heavy chain variable region comprising the amino acid sequence set forth in SEQ ID NO: 5.

In certain embodiments of the above aspects or any other aspect or embodiment disclosed herein, the antibody or antigen-binding fragment comprises a light chain comprising a light chain variable region comprising the amino acid sequence set forth in SEQ ID NO: 11.

In certain aspects, the invention provides an antibody or antigen-binding fragment thereof.

In certain embodiments of the above aspects or any other aspect or embodiment disclosed herein, the antibody or antigen-binding fragment comprises a heavy chain comprising a heavy chain variable region comprising the amino acid sequence set forth in SEQ ID NO: 6.

In certain embodiments of the above aspects or any other aspect or embodiment disclosed herein, the antibody or antigen-binding fragment comprises a light chain comprising a light chain variable region comprising the amino acid sequence set forth in SEQ ID NO: 11.

In certain embodiments of the above aspects or any other aspect or embodiment disclosed herein, the antibody or antigen-binding fragment thereof is selected from the group consisting of a full-length antibody, a Fab, and a single-chain variable fragment (scFv).

In certain embodiments of the above aspects or any other aspect or embodiment disclosed herein, the antibody or antigen-binding fragment thereof is a full-length antibody.

In certain embodiments of the above aspects or any other aspect or embodiment disclosed herein, the antibody is humanized.

In certain embodiments of the above aspects or any other aspect or embodiment disclosed herein, the heavy chain further comprises a constant domain of a human immunoglobulin heavy chain and the light chain further comprises a constant domain of a human light chain.

In certain embodiments of the above aspects or any other aspect or embodiment disclosed herein, the constant domain of a human immunoglobulin heavy chain is from an IgG1 heavy chain.

In certain embodiments of the above aspects or any other aspect or embodiment disclosed herein, the constant domain of a human immunoglobulin heavy chain comprises the amino acid sequence set forth in SEQ ID NO: 14.

In certain embodiments of the above aspects or any other aspect or embodiment disclosed herein, the constant domain of a human light chain is a from a human kappa light chain.

In certain embodiments of the above aspects or any other aspect or embodiment disclosed herein, the constant domain of a human light chain comprises the amino acid sequence set forth in SEQ ID NO: 15.

In certain aspects, the invention provides an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising at least about 85%, 90%, 91%, 92%, 93%, 94%, 95%, 96%, 97%, 98%, or 99% sequence identity to the amino acid sequence set forth in SEQ ID NO: 38.

In certain embodiments of the above aspects or any other aspect or embodiment disclosed herein, the antibody or antigen-binding fragment comprises a light chain comprising at least about 85%, 90%, 91%, 92%, 93%, 94%, 95%, 96%, 97%, 98%, or 99% sequence identity to the amino acid sequence set forth in SEQ ID NO: 40.

In certain embodiments of the antibody or antigen-binding fragment of the above aspect or any other aspect or embodiment disclosed herein, the variable region of the heavy chain comprises a HCDR1 comprising the amino acid sequence set forth in SEQ ID NO: 16, a HCDR2 comprising the amino acid sequence set forth in SEQ ID NO: 17, and a HCDR3 comprising the amino acid sequence set forth in SEQ ID NO: 20.

In certain embodiments of the above aspects or any other aspect or embodiment disclosed herein, the variable region of the light chain comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain is set forth in SEQ ID NO: 38 and the light chain is set forth in SEQ ID NO: 40.

In certain aspects, the invention includes an antibody or antigen-binding fragment thereof that specifically binds to an epitope of human EPH Receptor A5.

In certain embodiments of the above aspects or any other aspect or embodiment disclosed herein, the antibody or antigen-binding fragment comprises a heavy chain comprising at least 85%, 90%, 91%, 92%, 93%, 94%, 95%, 96%, 97%, 98%, or 99% sequence identity to the amino acid sequence set forth in SEQ ID NO: 39.

In certain embodiments of the above aspects or any other aspect or embodiment disclosed herein, the antibody or antigen-binding fragment comprises a light chain comprising at least about 85%, 90%, 91%, 92%, 93%, 94%, 95%, 96%, 97%, 98%, or 99% sequence identity to the amino acid sequence set forth in SEQ ID NO: 40.

In certain embodiments of the antibody or antigen-binding fragment of the above aspects or any other aspect or embodiment disclosed herein, the variable region of the heavy chain comprises a HCDR1 comprising the amino acid sequence set forth in SEQ ID NO: 16, a HCDR2 comprising the amino acid sequence set forth in SEQ ID NO: 18, and a HCDR3 comprising the amino acid sequence set forth in SEQ ID NO: 20.

In certain embodiments of the above aspects or any other aspect or embodiment disclosed herein, the variable region of the light chain comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

In certain embodiments of the above aspects or any other aspect or embodiment disclosed herein, the heavy chain is set forth in SEQ ID NO: 39 and the light chain is set forth in SEQ ID NO: 40.

In certain embodiments of the above aspects or any other aspect or embodiment disclosed herein, the dissociation constant (K_{D}) for binding to human EpHA5 is less than 1.25×10⁻⁹ M.

In certain embodiments of the above aspects or any other aspect or embodiment disclosed herein, the dissociation constant (K_{D}) for binding to human EpHA5 is between 8×10⁻¹⁰ M and 1.1×10⁻⁹ M.

In certain embodiments of the above aspects or any other aspect or embodiment disclosed herein, the antibody or antigen-binding fragment has a dissociation constant (K_{D}) for binding to human EpHA5 that is within 2-fold better than a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the dissociation constant (K_{D}) for binding to human EpHA5 is more than 1.2 fold better than a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the dissociation constant (K_{D}) is determined by surface plasmon resonance, or “SPR” analysis.

In certain embodiments of the above aspects or any other aspect or embodiment disclosed herein, the antibody or antigen-binding fragment of any of the above aspects or any other aspect or embodiment disclosed herein exhibits increased thermal stability compared to a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the antibody or antigen-binding fragment of any of the above aspects or any other aspect or embodiment disclosed herein has a melting temperature profile with a Tm1 that is increased greater than at or about 5° C. compared to a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the antibody or antigen-binding fragment of any of the above aspects or any other aspect or embodiment disclosed herein has a melting temperature profile with a Tm1 that is increased greater than about 10° C. compared to a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the Tm1 of the thermal unfolding curve of the antibody or antigen-binding fragment is greater than 60° C.

In certain embodiments of the above aspects or any other aspect or embodiment disclosed herein, the Tm1 of the antibody or antigen-binding fragment is between about 60° C. and about 70° C.

In certain embodiments of the above aspects or any other aspect or embodiment disclosed herein, the Tm1 of the antibody or antigen-binding fragment is about 64° C., about 65° C., about 66° C., about 67° C., about 68° C., or about 69° C.

In certain embodiments of the above aspects or any other aspect or embodiment disclosed herein, the melting temperature profile is monophasic.

In certain embodiments of the above aspects or any other aspect or embodiment disclosed herein, the onset temperature of aggregation (Tagg) is increased greater than about 1° C., greater than about 2° C., greater than about 3° C., greater than about 4° C., or greater than about 5° C. compared to a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the onset temperature of aggregation (Tagg) is greater than about 67° C.

In certain embodiments of the above aspects or any other aspect or embodiment disclosed herein, the onset temperature of aggregation (Tagg) is between about 67° C. and about 71° C.

In certain embodiments the Tagg of the antibody or antigen-binding fragment is about 67° C., about 68° C., about 69° C., about 70° C., or about 71° C.

In certain embodiments of the above aspects or any other aspect or embodiment disclosed herein, the antibody or antigen-binding fragment binds to cell surface human EphA5.

In certain embodiments of the above aspects or any other aspect or embodiment disclosed herein, the antibody or fragment binds human EphA5 expressed on the surface of cells, optionally an H460 cell line, with an EC50 that is increased compared to a reference antibody, wherein the reference antibody is 11C12 or an antibody comprising a variable heavy chain set forth in SEQ ID NO: 1 and a variable light chain set forth in SEQ ID NO: 8, optionally wherein the reference antibody is of the same form.

In certain embodiments of the above aspects or any other aspect or embodiment disclosed herein, the antibody or fragment binds human EphA5 expressed on the surface of cells, optionally an H460 cell line, with an EC50 of less than or equal to 0.020 μg/mL optionally wherein the EC50 is determined by flow cytometry.

In certain embodiments of the above aspects or any other aspect or embodiment disclosed herein, the antibody or fragment binds human EphA5 expressed on the surface of cells, optionally an H460 cell line, with an EC50 of between about 0.010 μg/mL and 0.020 μg/mL, optionally wherein the EC50 is determined by flow cytometry.

In certain embodiments of the above aspects or any other aspect or embodiment disclosed herein, the antibody or fragment binds human EphA5 expressed on the surface of cells, optionally an H460 cell line, with an EC50 of about 0.015 μg/mL, about 0.016 μg/mL, about 0.017 μg/mL, about 0.018 μg/mL, about 0.019 μg/mL, or about 0.020 μg/mL, optionally wherein the EC50 is determined by flow cytometry.

In certain embodiments of the above aspects or any other aspect or embodiment disclosed herein, the antibody or antigen-binding fragment is internalized by human EphA5-expressing cells.

In certain aspects, the invention includes a nucleic acid encoding the heavy chain of the antibody or antigen-binding fragment thereof of any one of the above aspects or any other aspect or embodiment disclosed herein.

In certain aspects, the invention includes a nucleic acid encoding the light chain of the antibody or antigen-binding fragment thereof of any one of the above aspects or any other aspect or embodiments disclosed herein.

In certain aspects, the invention includes a nucleic acid encoding the heavy chain and the light chain of the antibody or antigen-binding fragment thereof of any one of the above aspects or any other aspect or embodiments disclosed herein.

In certain aspects, the invention includes a vector comprising the nucleic acid of any one of the above aspects or any other aspect or embodiments disclosed herein.

In certain embodiments of the above aspects or any other aspect or embodiment disclosed herein, the vector is an expression vector.

In certain aspects, the invention includes a vector comprising a nucleic acid encoding the heavy chain and a nucleic acid encoding the light chain, wherein the heavy chain and light chain is of the antibody or antigen-binding fragment of any one of the above aspects or any other aspect or embodiments disclosed herein.

In certain embodiments of the above aspects or any other aspect or embodiment disclosed herein, the vector is a bicistronic vector.

In certain aspects, the invention includes a vector system, comprising a first vector comprising a first nucleic acid encoding a heavy chain and a second vector comprising a second nucleic acid encoding a light chain, wherein the heavy chain and the light chain is of the antibody or antigen-binding fragment of any one of the above aspects or any other aspect or embodiments disclosed herein.

In certain embodiments of the above aspects or any other aspect or embodiment disclosed herein, the first vector and the second vector are each an expression vector.

In certain aspects, the invention includes a host cell comprising the vector of any one of the above aspects or any other aspect or embodiments disclosed herein.

In certain embodiments of the above aspects or any other aspect or embodiment disclosed herein, the host cell is a mammalian cell.

In certain aspects, the invention includes a method of producing an antibody, comprising introducing a vector of any one of the above aspects or any other aspect or embodiments disclosed herein into a host cell, culturing the host cell under conditions for expression of an antibody or antigen binding fragment from the host cell, and isolating or purifying the antibody or antigen-binding fragment.

In certain embodiments of the above aspects or any other aspect or embodiment disclosed herein, the host cell is a mammalian cell.

In certain aspects of this disclosure, the host cell is a human cell.

In certain aspects, this disclosure includes an immunoconjugate having the formula Ab-(L-D)x, with the terms “Ab”, “L” and “D” as defined herein.

In certain aspects of this disclosure, Ab of the formula Ab-(L-D)x is an antibody or antigen-binding fragment thereof that selectively binds to EphA5, such as the antibodies disclosed herein. In certain aspects of this disclosure, Ab is an antibody that specifically binds to an epitope of EphA5, the Ab comprising the heavy chain sequence set forth in SEQ ID NO: 6 and the light chain sequence set forth in SEQ ID NO: 11. In certain aspects of this disclosure, Ab is an antibody that specifically binds to an epitope of EphA5, the Ab comprising the heavy chain sequence set forth in SEQ ID NO: 39 and the light chain sequence set forth in SEQ ID NO: 40. In certain aspects of this disclosure, Ab is an antibody that specifically binds to an epitope of EphA5, the Ab comprising the heavy chain sequence set forth in SEQ ID NO: 38 and the light chain sequence set forth in SEQ ID NO: 40. In aspects of this disclosure, Ab is an antibody that specifically binds to an epitope of EphA5, the Ab comprising the heavy chain CDR sequences set forth in SEQ ID NOs: 16, 18 and 20, and the light chain sequences set forth in SEQ ID NOs: 21, 23 and 24.

In certain aspects, the antibodies described herein and above, and the ADC comprising these antibodies, comprise sequences having at least 90% sequence identity to the amino acid sequences disclosed herein and above.

In certain aspects of this disclosure, L is a linker. Linkers (“L”) and linker drug constructs “L-D” as used herein are in some cases presented as linker reagents, i.e., linker reagents have reactive moieties which can react with, and covalently bond to, an appropriate atom or atoms of the antibody or antigen binding fragment. Ab-(L-D)x and Ab-L-D refer to an antibody drug conjugate (ADC) where one or more reactive linker moieties have reacted with and formed one or more covalent bonds with an atom or atoms of an antibody or antigen-binding fragment.

In certain aspects of this disclosure, the linker L contains a cleavable linker moiety.

In certain embodiments of the above aspects or any other aspect or embodiment disclosed herein of this disclosure, the cleavable linker moiety is a tetrapeptide.

In certain aspects of this disclosure, the linker L is a cathepsin-cleavable linker moiety.

In certain embodiments of the above aspects or any other aspect or embodiment disclosed herein of this disclosure, the cathepsin-cleavable linker moiety comprises a valine-citrulline (Val-Cit).

In certain aspects of this disclosure, the linker L comprises a self-immolative linker, see, for example, Edupuganti, V et al. Recent Patents on Anti-Cancer Drug Discovery 16.4 (2021): 479-497.)

In certain embodiments of the above aspects or any other aspect or embodiment disclosed herein of this disclosure, the self-immolative linker moiety comprises p-aminobenzyloxycarbonyl (PAB).

In certain embodiments of the above aspects or any other aspect or embodiment disclosed herein of this disclosure, the linker also includes a polyethylene glycol (“PEG”) chain.

In certain embodiments of the above aspects or any other aspect or embodiment disclosed herein of this disclosure, the PEG chain is composed of from 24 to 6 polyethylene glycol (—CH₂—CH₂—O—) units.

In certain aspects of this disclosure, the linker L comprises two or more of the aspects or embodiments described herein.

In certain aspects of this disclosure, the linker reagent is MC-Val-Cit-PAB, which incorporates the MC (maleimidocaproyl) reactive moiety:

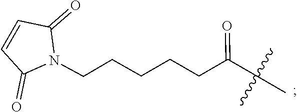 [SMILES: *C(=O)CCCCCN1C(=O)C=CC1=O]

the Val-Cit (valine-citrulline) cleavable moiety:

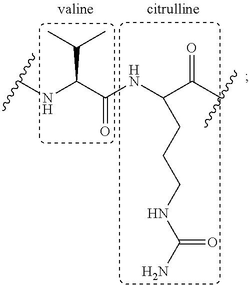 [SMILES: *N[C@H](C(=O)NC(CCCNC(N)=O)C(*)=O)C(C)C]

and the PAB (p-aminobenzyloxycarbonyl) moiety:

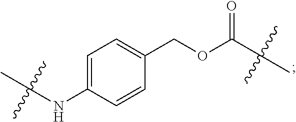 [SMILES: *Nc1ccc(COC(*)=O)cc1]

to form the linker reagent MC-Val-Cit-PAB having the structure:

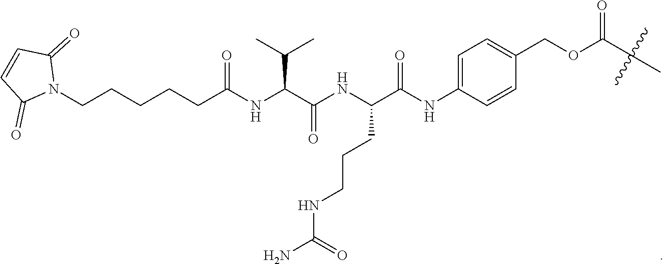 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

wherein the frilled line indicates the attachment point of the cytotoxic drug D.

In certain embodiments of the above aspects or any other aspect or embodiment disclosed herein, the linker reagent is TBR-Glu-(Val-Cit-PAB)-amino-PEG₂₄-Me, having the structure:

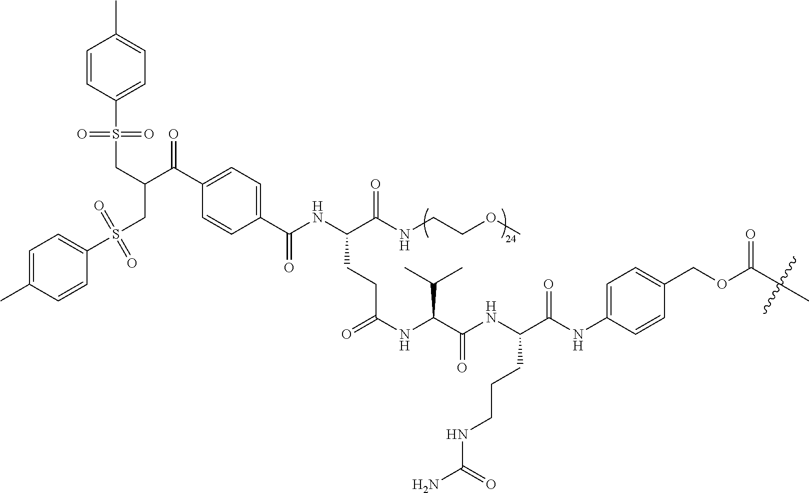

or TBR-[Glu-(Val-Cit-PAB)]₂-Glu-(amino-PEG₂₄-Me)₂, having the structure:

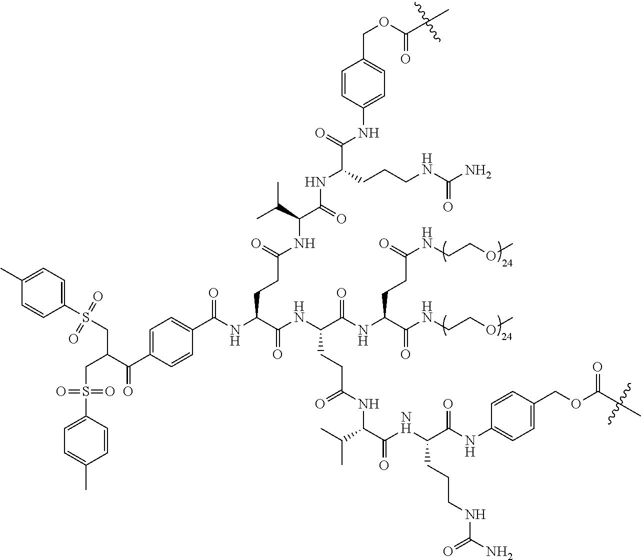

wherein the frilled line in each instance indicates the attachment point of the cytotoxic drug D.

In certain embodiments of the above aspects or any other aspect or embodiment disclosed herein, the linker is a pH cleavable linker.

In certain embodiments of the above aspects or any other aspect or embodiment disclosed herein, the linker is CL2A, having the structure:

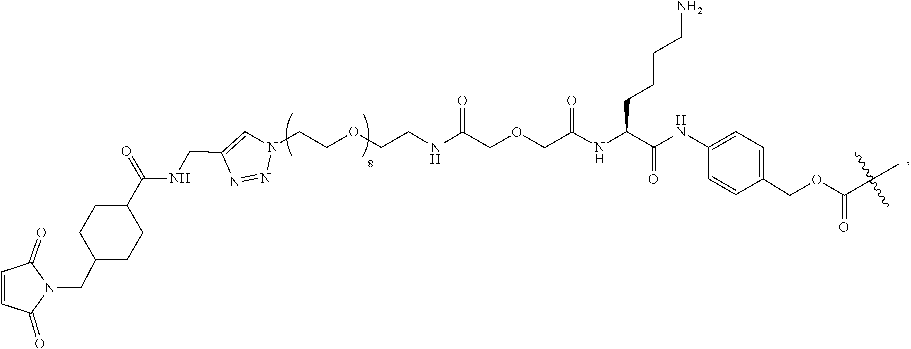

wherein the frilled line indicates the attachment point of the cytotoxic drug D.

In certain embodiments of the above aspects or any other aspect or embodiment disclosed herein, the cytotoxic drug is an auristatin.

In certain aspects, the cytotoxic drug D is an auristatin or an auristatin analog.

In certain embodiments of the above aspects or any other aspect or embodiment disclosed herein, the auristatin is monomethyl auristatin E (MMAE) having the structure:

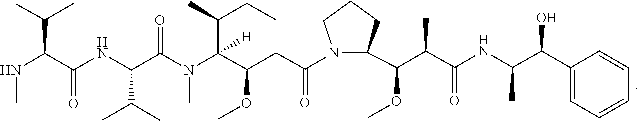 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In certain embodiments of the above aspects or any other aspect or embodiment disclosed herein, the L-D reagent is MC-Val-Cit-PAB-MMAE having the structure:

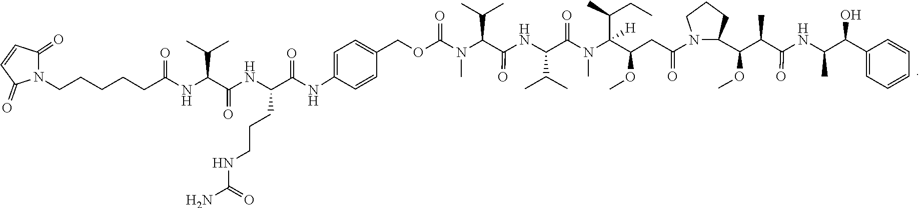 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In certain embodiments of the above aspects or any other aspect or embodiment disclosed herein, the L-D reagent is TBR-Glu-(Val-Cit-PAB-MMAE)-amino-PEG₂₄-Me, having the structure:

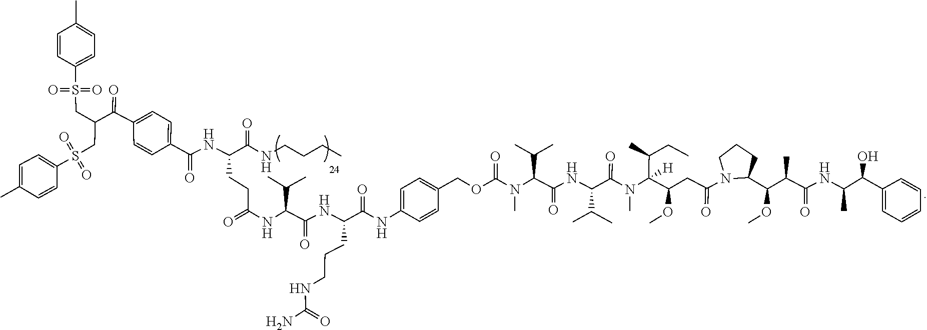

In certain aspects, the invention includes an immunoconjugate having the formula Ab-(L-D).

In certain embodiments of the above aspects or any other aspect or embodiment disclosed herein, Ab is an antibody that specifically binding to an epitope of human EPH Receptor A5 (EphA5) comprising the heavy chain set forth in SEQ ID NO: 38 and the light chain set forth in SEQ ID NO: 40, the heavy chain set forth in SEQ ID NO: 39 and the light chain set forth in SEQ ID NO: 40, the heavy chain set forth in SEQ ID NO: 6 and the light chain set forth in SEQ ID NO: 11, as described in greater detail herein and below.

In certain embodiments of the above aspects or any other aspect or embodiment disclosed herein, Ab is an antibody that specifically binding to an epitope of human EPH Receptor A5 (EphA5) comprising:
(a) a first VH CDR set forth in SEQ ID NO: 16;
(b) a second VH CDR set forth in SEQ ID NO: 18;
(c) a third VH CDR set forth in SEQ ID NO: 20;
(d) a first VL CDR set forth in SEQ ID NO: 21;
(e) a second VL CDR set forth in SEQ ID NO: 23; and
(f) a third VL CDR set forth in SEQ ID NO: 24.

In certain aspects, the invention includes an immunoconjugate having the formula Ab-(L-D) and the bridged disulfide structure:

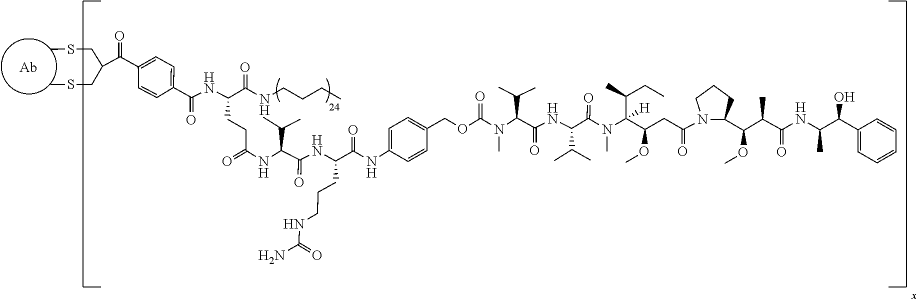

wherein x is from 1 to 8, and wherein the antibody (Ab) is as described herein and below, and in particular as described immediately above.

In certain embodiments of the above aspects or any other aspect or embodiment disclosed herein, the L-D is TBR-Glu-(Val-Cit-PAB-MMAE)-amino-PEG₂₄-Me, having the structure:

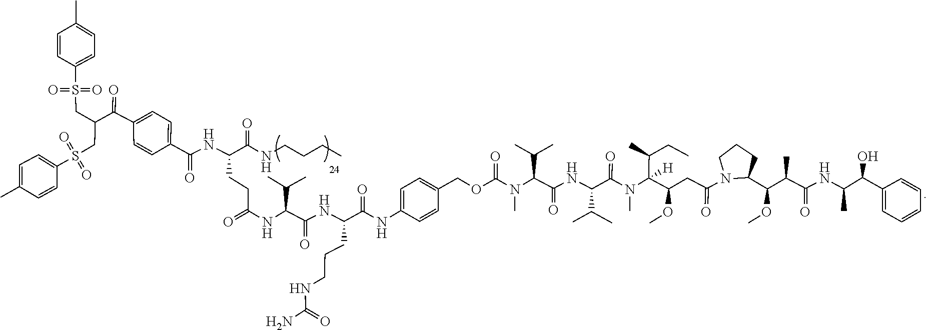

In certain embodiments of the above aspects or any other aspect or embodiment disclosed herein, the cytotoxic drug is SN38, having the structure:

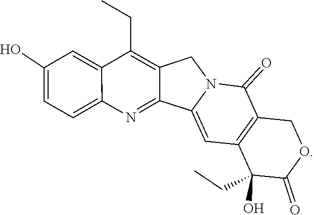 [SMILES: CCc1c2c(nc3ccc(O)cc13)-c1cc3c(c(=O)n1C2)COC(=O)[C@]3(O)CC]

In certain embodiments of the above aspects or any other aspect or embodiment disclosed herein, the L-D reagent is CL2A-SN38, having the structure:

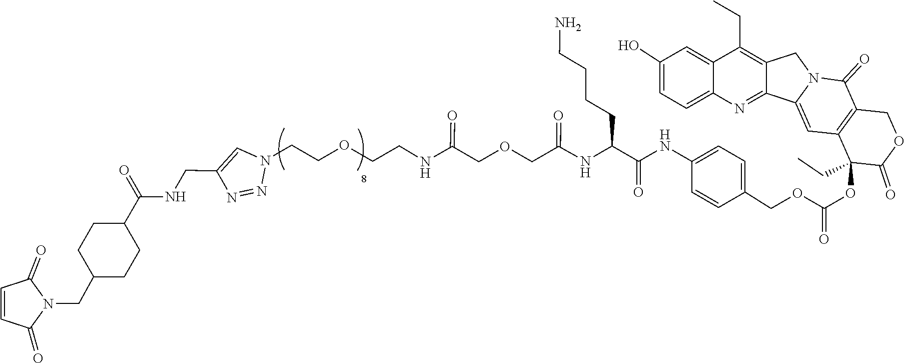

In certain embodiments of the above aspects or any other aspect or embodiment disclosed herein, the L-D reagent is TBR-[Glu-(Val-Cit-PAB-SN38)]₂-Glu-(amino-PEG₂₄-Me)₂, having the structure:

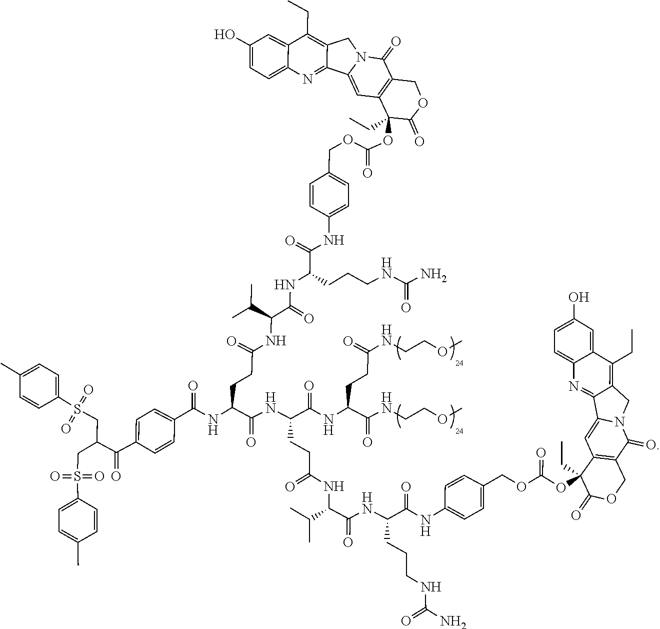

In certain aspects, this disclosure includes an immunoconjugate having the formula Ab-(L-D)x.

In certain aspects of this disclosure, the drug to antibody ratio (“DAR”) is or is from about 1 to about 8.

In certain embodiments of the above aspects or any other aspect or embodiment disclosed herein, the drug to antibody ratio is or is about 4.

In certain embodiments of the above aspects or any other aspect or embodiment disclosed herein, the drug to antibody ratio is or is about 8.

In certain aspects, the invention includes a pharmaceutical composition comprising the antibody or antigen-binding fragment of any one of the above aspects or any aspect or embodiment disclosed herein and a pharmaceutically acceptable carrier.

In certain aspects, the invention includes a pharmaceutical composition comprising the immunoconjugate of any one of the above aspects or any aspect or embodiment disclosed herein and a pharmaceutically acceptable carrier.

In certain aspects, the invention includes a method for treating, ameliorating, and/or preventing cancer in a subject in need thereof, the method comprising administering to the subject an effective amount of the antibody or antigen-binding fragment thereof of the above aspects or any aspect or embodiment disclosed herein.

In certain aspects, the invention includes a method for treating, ameliorating, and/or preventing cancer in a subject in need thereof, the method comprising administering to the subject an effect amount of the immunoconjugate of any one of the above aspects or any aspect or embodiment disclosed herein.

In certain aspects, the invention includes a method for treating, ameliorating, and/or preventing cancer in a subject in need thereof, the method comprising administering to the subject an effect amount of the pharmaceutical composition of the above aspects or any aspect or embodiment disclosed herein.

In certain aspects, the invention includes a method for treating, ameliorating, and/or preventing cancer in a subject in need thereof, the method comprising administering to the subject an effect amount of the pharmaceutical composition of the above aspects or any aspect or embodiment disclosed herein.

In certain aspects, the invention includes a method for inducing tumor regression in a subject, the method comprising administering to the subject an effective amount of the antibody or antigen-binding fragment thereof of any one of the above aspects or any aspect or embodiment disclosed herein.

In certain aspects, the invention includes a method for inducing tumor regression in a subject in need thereof, the method comprising administering to the subject an effect amount of the immunoconjugate of any one of the above aspects or any aspect or embodiment disclosed herein.

In certain aspects, the tumor is a solid tumor. In certain aspects, the tumor is a liquid tumor. In certain aspects, the liquid tumor is a blood cancer.

In certain aspects, the invention includes a method for inducing tumor regression in a subject in need thereof, the method comprising administering to the subject an effect amount of the pharmaceutical composition of the above aspects or any aspect or embodiment disclosed herein.

In certain aspects, the invention includes a method for inducing tumor regression in a subject in need thereof, the method comprising administering to the subject an effect amount of the pharmaceutical composition of the above aspects or any aspect or embodiment disclosed herein.

In certain embodiments of the above aspects or any other aspect or embodiment disclosed herein, the cancer is associated with expression of EPH Receptor A5.

In certain embodiments of the above aspects or any other aspect or embodiment disclosed herein, the tumor is associated with expression of EPH Receptor A5.

In certain embodiments of the above aspects or any other aspect or embodiment disclosed herein, the tumor is a cancer.

In certain embodiments of the above aspects or any other aspect or embodiment disclosed herein, the EPH Receptor A5 is expressed on the cancer cells.

In certain aspects of this disclosure, the EphA5 is differentially (e.g., to a greater extent) expressed on the cancer cells compared to normal cells.

In certain embodiments of the above aspects or any other aspect or embodiment disclosed herein, the cancer is a carcinoma

In certain embodiments of the above aspects or any other aspect or embodiment disclosed herein, the cancer is squamous cell carcinoma.

In certain embodiments of the above aspects or any other aspect or embodiment disclosed herein, the cancer is head and neck squamous cell carcinoma.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 illustrates the structure of TBR-Glu-(Val-Cit-PAB-MMAE)-amino-PEG₂₄-Me linker-payload, comprising the Val-Cit-PAB linker, the cytotoxic payload MMAE (on one side), and the site-specific Abzena ThioBridge® (“TBR”) conjugation technology on the other side. This linker-payload is typically conjugated to an antibody comprising VH5VK3 as described herein.

FIG. 2 illustrates IHC staining of SCC tissue from the TM01141 PDX model using a control IgG antibody. No discernible EphA5 cell surface expression was seen on the cell surface of the cell in the tissue sample.

FIG. 3 illustrates IHC staining of SCC tissue from the TM01141 PDX model using a commercially available antibody that selectively binds to EphA5. Cell surface expression of EphA5 was detected on the TM01141 tumors.

FIG. 4 is a table summarizing cell staining and H-scores for tumor tissue from the TM01141 PDX model.

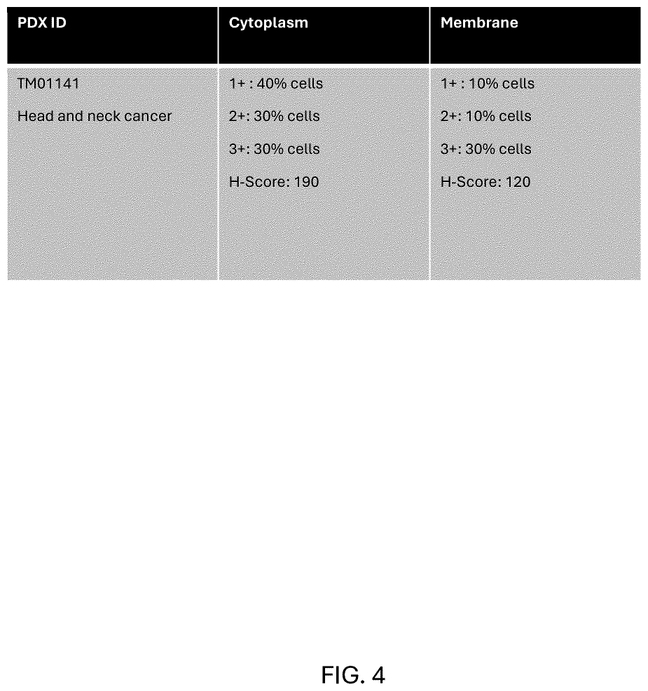

FIG. 5 is a line graph showing tumor volume in TM01141 PDX mice treated weekly for 5 weeks with different dose levels of TA1 (MBRC-101) or placebo, measured over a 50-day test period.

FIG. 6 is a bar graph showing weights of tumors removed from PDX-TM01141-bearing mice following treatment with TA1 (MBRC-101) or placebo.

FIG. 7 comprises bar graphs (FIGS. 7A-7D) showing body weights of PDX-TM01141-bearing mice before and after treatment with TA1 (MBRC-101).

FIG. 8 is a line graph showing tumor volume in TM00375 mice treated weekly for three weeks with different dose levels of TA1 (MBRC-101) or placebo, measured over a 32-day test period.

FIG. 9 is a line graph showing tumor volume in TM00980 mice treated weekly for three weeks with TA1 (MBRC-101), irinotecan or placebo, measured over a 32-day test period.

FIG. 10 is a line graph showing tumor volume in TM01212 mice treated with one dose of TA1 (MBRC-101), two weekly doses of TA2 (datopotamab deruxtecan) or placebo, measured over a 30-day period.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “recombinant,” as used herein, refers to nucleic acid or protein molecules formed by artificial (e.g. laboratory-derived) methods of genetic engineering (e.g. molecular cloning) that bring together genetic sequences from multiple sources, thus creating sequences that would not otherwise be found in natural genomes.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules comprising two heavy chain and two light chain polypeptides. Each polypeptide chain contains three complementarity-determining regions (CDRs), which bind to the antigen and defines the antibody's antigen specificity.

As used herein, the term “antibody” and “antibodies” can also include polypeptides or polypeptide complexes derived from full-length antibodies. These polypeptide complexes may be naturally occurring or constructed from single chain antibodies or antibody fragments and retain an antigen-specific binding ability. The antibodies of the present invention may exist in a variety of forms including, for example, polyclonal antibodies, monoclonal antibodies, Fv, Fab and F(ab′)₂, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). “Antibody” also includes single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

The term “antibody fragment” refers to a polypeptide comprising or derived from a portion of an intact antibody and may comprise the antigen-binding fragment of an intact antibody.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. K and k light chains refer to the two major antibody light chain isotypes.

The term “specifically binds” to an antigen or epitope is a term that is well understood in the art, and methods to determine such specific binding are also well known in the art. A binding molecule, such as an antibody or antigen-binding fragment, is said to exhibit “specific binding,” such as “preferential binding” if it reacts or associates more frequently, more rapidly, with greater duration and/or with greater affinity with a particular target than it does with alternative targets. An antibody specifically binds or preferentially binds to a target if it binds with greater affinity, avidity, more readily, and/or with greater duration than it binds to other substances. For example, an antibody that specifically or preferentially binds to an EphA8 is an antibody that binds EphA8 with greater affinity, avidity, more readily, and/or with greater duration than it binds to a non-target protein epitope. It is also understood by reading this definition that; for example, a binding molecule that specifically or preferentially binds to a first target may or may not specifically or preferentially bind to a second target. It is also understood by reading this definition that specific binding or preferential binding does not necessarily require (although it can include) exclusive binding. Methods to determine such specific or preferential binding are also well known in the art, e.g., an immunoassay.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “antibody-drug conjugate” or “ADC” as used herein refers to an antibody-based molecular complex comprising an antibody or antigen-binding polypeptide fragment derived from an antibody conjugated to a biologically active drug molecule, often referred to as the “payload” but also herein as “drug” or “D”. ADC-associated drug molecules are often cytotoxic in function, which allows the ADC complex to kill cells expressing specific molecular targets, especially tumor cells.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “Kd”, “K_{D}” or “equilibrium dissociation constant,” as used herein, refers to a calculated, quantitative measurement of an antibody's affinity for its epitope. The K_{D} represents a ratio of the rate of binding (Kon) and dissociation (Koff) between the antibody and its epitope. K_{D} and affinity are inversely related in that a high affinity interaction is characterized by a low K_{D}.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer (including triple negative and HR+/HER2−), prostate cancer, ovarian cancer (including ovarian adenocarcinoma), cervical cancer (including squamous cell cervical cancer), skin cancer (including squamous cell skin cancer), pancreatic cancer (including pancreatic adenocarcinoma), colorectal cancer, renal cancer, liver cancer (including hepatocellular carcinoma), brain cancer, lymphoma, leukemia, lung cancer (including NSCLC), gastric (stomach) cancer (including gastric adenocarcinoma), testicular cancer, bone cancer, esophageal cancer (including squamous cell esophageal cancer), thyroid cancer, urothelial cancer, and head and neck cancer (including HNSCC), to name some of the more common types of cancer, but also includes less common and rare forms of cancer, and sub-types of the aforementioned cancer indications.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Exemplary conservative substitutions are shown below:

| | | Preferred Conservative |
|---|---|---|
| Original Amino Acid | Exemplary Conservative Substitutions of | Substitution of the Original |
| Residues in SEQ ID | the Original Amino Acid Residues of | Amino Acid Residues of |
| NOs. 1-24 and 38-42 | SEQ ID NOs. 1-24 and 38-42 | SEQ ID NOs. 1-24 and 38-42 |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4 Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

Thus, one or more amino acid residues within the CDR regions of an antibody of the invention can be replaced with other amino acid residues from the same side chain family and the altered antibody can be tested for the ability to bind epitopes such as EPH receptor A5 using the functional assays described herein.

The term “dysregulated” when used in the context of the level of expression or activity of EphA5 refers to the level of expression or activity that is different from the expression level or activity of EphA5 in an otherwise identical healthy animal, organism, tissue, cell or component thereof. The term “dysregulated” also refers to the altered regulation of the level of expression and activity of EphA5 compared to the regulation in an otherwise identical healthy animal, organism, tissue, cell or component thereof. In certain embodiments, the dysregulation of expression or activity is associated with a disease state including cancer in which expression level or activity of EphA5 is dysregulated within cancer cells.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Linker-Drug reagent” or “L-D reagent” refers to a linker drug moiety having a reactive group or groups that effect(s) formation covalent bonds between an L-D and a suitable reactive group or groups of an antibody to form an ADC. In the case of ThioBridge® (TBR”) technology two toluene sulfinate groups are eliminated in the formation of a bridged disulfide with the antibody.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” and “chimeric” forms of non-human (e.g., murine) antibodies are immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized and chimeric antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized and chimeric antibodies can comprise residues which are found neither in the recipient antibody nor in the imported CDR or framework sequences. These modifications are made to further refine and optimize antibody performance. In general, the humanized and chimeric antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized and chimeric antibody optimally also will comprise at least a portion of an immunoglobulin constant region (Fc), typically that of a human immunoglobulin.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

In certain embodiments, a nucleic acid molecule encoding one or more binding agents described herein may be inserted into one or more expression vectors, as discussed below in greater detail. In such embodiments, the binding agent may be encoded by nucleotides corresponding to the amino acid sequence. The particular combinations of nucleotides (codons) that encode the various amino acids (AA) are well known in the art, as described in various references used by those skilled in the art (e.g., Lewin, B. Genes V, Oxford University Press, 1994). The nucleotide sequences encoding the amino acids of said binding agents may be ascertained with reference to the table provided below, for example. Nucleic acid variants may use any combination of nucleotides that encode the binding agent.

### Codons Encoding Amino Acids (AA) of SEQ ID NOS. 1-24 and 38-42 or Variants Thereof

| | Co- | | Co- | | Co- | | Co- |
|---|---|---|---|---|---|---|---|
| AA | don | AA | dons | AA | dons | AA | dons |
| Phe (F) | TTT | Ser (S) | TCT | Tyr (Y) | TAT | Cys (C) | TGT |
| | TTC | | TCC | | TAC | | TGC |
| Leu (L) | TTA | | TCA | TERM | TAA | TERM | TGA |
| | TTG | | TCG | | TAG | Trp (W) | TGG |
| | CTT | Pro (P) | CCT | His (H) | CAT | Arg (R) | CGT |
| | CTC | | CCC | | CAC | | CGC |
| | CTA | | CCA | Gln (Q) | CAA | | CGA |
| | CTG | | CCG | | CAG | | CGG |
| Ile (I) | ATT | Thr (T) | ACT | Asn (N) | AAT | Ser (S) | AGT |
| | ATC | | ACC | | AAC | | AGC |
| | ATA | | ACA | Lys (K) | AAA | Arg (R) | AGA |
| Met (M) | ATG | | ACG | | AAG | | AGG |
| Val (V) | GTT | Ala (A) | GCT | Asp (D) | GAT | Gly (G) | GGT |
| | GTC | | GCC | | GAC | | GGC |
| | GTA | | GCA | Glu (E) | GAA | | GGA |
| | GTG | | GCG | | GAG | | GGG |

Those of ordinary skill in the art understand that a nucleotide sequence encoding a particular amino acid sequence may be easily derived from the amino acid sequences of SEQ ID NOS: 1-24 and 38-42. For instance, it may be deduced from the amino acid sequence of Chimeric VHO Variable Heavy Chain: EVTLKESGGGLVQPGGSRKLSCAASGFTFSTFGIHWVRQAPEKGLEWVAYISGASTTI YYADTVKGRFTISRDNPKNTLFLQMTSLRSEDTAMYYCARYGTSFPYGLDYWGQGT SVTVSS (SEQ ID NO: 1) and the information presented in the above table that the amino acid sequence may be encoded by the nucleotide sequence: GAAGTGACCCTGAAAGAGTCTGGCGGAGGACTGGTTCAGCCTGGCGGATCTAGA AAGCTGTCTTGTGCCGCCTCCGGCTTCACCTTCTCTACCTTTGGCATCCACTGGGT CCGACAGGCCCCTGAGAAAGGATTGGAGTGGGTCGCCTACATCTCCGGCGCTTC CACCACCATCTACTACGCCGATACCGTGAAGGGCAGATTCACCATCAGCCGGGA CAACCCCAAGAACACCCTGTTTCTGCAGATGACCAGCCTGCGGAGCGAGGACAC CGCCATGTACTACTGTGCCAGATACGGCACCAGCTTTCCCTACGGCCTGGATTAT TGGGGCCAGGGAACCTCCGTTACAGTCTCCTCA (SEQ ID NO: 25) (see, e.g., the tool provided at https://www.bioinformatics.org/sms2/rev_trans.html). Those of ordinary skill in the art would understand that nucleotide sequences encoding any of SEQ ID NOS. 1-24 and 38-42 may be deduced in the same way, and such nucleotide sequences are contemplated herein. Where the binding agents are antibodies, nucleotide sequences encoding the variable regions thereof may also be isolated from the phage and/or hybridoma cells expressing the same cloned into expression vectors to produce antibody preparations.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encoded or specified by a gene, causes the gene product to be produced in a cell substantially only if the cell is a cell of the tissue type corresponding to the promoter.

“Single chain antibodies” refer to antibodies formed by recombinant DNA techniques in which immunoglobulin heavy and light chain fragments are linked to each other using an engineered span of amino acids to recapitulate the Fv region of an antibody as a single polypeptide. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “Thiobridge”, “ThioBridge®”, “ThioBridge linker” or “TBR” or “TB®” as used herein means 4-(3-tosyl-2-(tosylmethyl)propanoyl)benzoic acid:

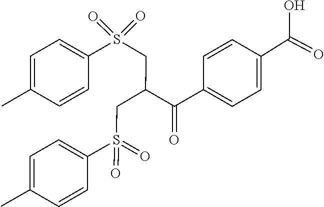 [SMILES: Cc1ccc(S(=O)(=O)CC(CS(=O)(=O)c2ccc(C)cc2)C(=O)c2ccc(C(=O)O)cc2)cc1]

and its derivatives. A ThioBridge linker or a derivative thereof, optionally when combined with other linker moieties, may be a component of a linker or a linker reagent.

The term “ThioBridge linear linker” or “TBLR” or “TBL®” as used herein means 4-[3-(Me-(PEG)₇)sulfonyl-2-[(Me-(PEG)₇))sulfonylmethyl]propanoyl]benzoic acid:

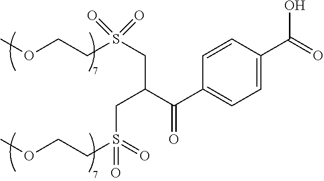

and its derivatives. A ThioBridge linear linker or a derivative thereof, optionally when combined with other linker moieties, may be a component of a linker or a linker reagent.

It is to be noted that a derivative of the ThioBridge linker and/or the ThioBridge linear linker, as employed in ADCs disclosed and claimed herein may comprise the moiety:

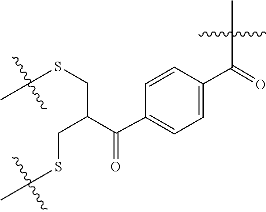 [SMILES: *SCC(CS*)C(=O)c1ccc(C(*)=O)cc1]

or a similar moiety, where the sulfurs are bonded to an antibody, most typically at a cysteine, and the carbonyl is bound to other linker components or a payload.

The term “treating” as used herein can refer to medical interventions performed with the purpose of ameliorating or preventing a disease state. The term refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof.

The Kabat and Clothia numbering systems may also be used to determine the position of other features of antibodies or antibody-derived fragments such as framework regions (FRs) and the like.

The term “thermal melting point” or “thermal melting temperature”, often abbreviated “Tm” or “Tmelt”, when used herein refers to the temperature at which a protein undergoes denaturation, an unfolding process in which quaternary, tertiary, and secondary structure are lost. The Tm of a protein can be measured directly via observing changes in intrinsic fluorescence due to tryptophan and tyrosine residues. Such Tm assays based on fluorescence can be used to rank the stability of protein constructs or compare different formulations to optimize various conditions. Other methods of measuring Tm are known in the art, and include but are not limited to static light scattering (SLS), dynamic light scattering (DLS), differential scanning calorimetry, circular dichromism, and the like and may be used to determine the Tm of the antibodies and antibody-derived fragments of the current invention.

The term “thermal aggregation” or “thermal aggregation temperature”, often abbreviated “Tagg” as used herein refers to the temperature at which protein molecules in suspension begin to oligomerize or aggregate. The temperature at which Tagg occurs depends on the physical properties of the protein itself and by the composition of the buffer solution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Provided herein are binding molecules, such as antibodies (including antigen-binding antibody fragments and immunoconjugates (e.g. antibody drug conjugates) that bind EphA5 and can be used to target EphA5 for treating, ameliorating, and/or preventing a disease or condition, such as a cancer. Also provided are nucleic acid molecules encoding the antibodies. Also provided are methods of making and using the antibodies and antigen-binding fragments as well as immunoconjugates (e.g. antibody drug conjugates) containing the antibodies and antigen-binding fragments. Also provided are compositions, including pharmaceutical compositions, containing such antibodies or antigen-binding fragments or immunoconjugates (e.g. antibody drug conjugates) containing the antibodies or antigen-binding fragments. In some aspects, the provided compositions, antibodies or antigen-binding fragments or immunoconjugates (e.g. antibody drug conjugates) containing the antibodies or antigen-binding fragments can be used in connection with a therapy or a method of treatment, amelioration, and/or prevention.

The present invention is based on the observation that humanized antibodies or antibody derived antigen-binding fragments specific for human EPH receptor A5 can be used to treat, ameliorate, and/or prevent cancers and diseases associated with the expression of EPH receptor A5. The antibodies of the invention can also be used in antibody drug conjugates useful for the same. Also provided are methods and compositions comprising the human EPH receptor A5 specific antibodies or antigen-binding fragments of the invention for the treatment, amelioration, and/or prevention of cancers.

EPH receptor A5, also referred to as ephrin type-A receptor 5 and EphA5, is protein encoded by the EPHA5 gene in humans and is a member of the ephrin receptor family of protein-tyrosine kinases. Receptors of this family are known to be involved in mediating signaling events primarily during development, especially in the nervous system. Ephrin family members including EPH receptor A5 typically possess a single kinase domain and an extracellular domain comprising a cystine-rich domain and two fibronectin type III repeats. Ephrin receptors are organized into two main groups based on the affinity their extracellular domain has for ephrin-A or ephrin-B ligands. Ephrin ligands, including the ligands for EPH receptor A5, share the distinctive property of being membrane-anchored. Ligands of the ephrin-A family are linked to the membrane via a GPI linkage, while ligands of the ephrin-B family are transmembrane proteins.

In human cancer, EPHA5 overexpression has been linked to, for example, breast, liver, brain, and lung cancers, where its inhibition generally leads to reduced growth of tumor cells. Recent studies in lung cancer identified that EPH receptor A5 is a key regulator of radiation resistance and treatment by activating and enhancing the DNA damage response (DDR). As such, the provided embodiments related to EPH receptor A5 inhibition, particularly by specific antibodies (e.g. the antibodies and antigen-binding fragments thereof of the invention), can slow tumor growth both alone and as a sensitization treatment in combination with other cytotoxic therapies and treatments. Thus, in certain aspects, the current invention provides human EPH receptor A5 antibodies and antigen-fragments thereof useful for the inhibition of EPH receptor A5 for the treatment of cancers. In certain embodiments, the EPH receptor A5 antibodies of the invention can be used in antibody drug conjugates in which the EPH receptor A5 specific antibodies are conjugated to cytotoxic agents that act to kill EPH receptor A5-expressing cells, especially cancer cells.

The provided antibodies (e.g. the antibodies and antigen-binding fragments thereof of the invention), as well as antibody drug conjugates containing the same, exhibit advantageous features compared to existing antibodies directed to EphA5 and immunoconjugates related to the same. In particular, the provided embodiments, produce a new antibody-based molecule that retains high affinity target binding for EphA5 with increased conformational stability, reduced immunogenicity risks and hence desirable manufacturable properties and features suitable for use as a therapeutic. In particular, the provided antibodies (e.g. the antibodies and antigen-binding fragments thereof) have been humanized rendering them suitable for administration to humans with reduced immunogenicity compared to other existing EphA5 antibodies, such as murine antibodies. Moreover, while humanization is known to impact certain properties of an antibody that could lead to reduced target binding affinity, aggregation, chemical stability, and/or physical stability during drug production and delivery, antibodies provided herein have been found to exhibit one or more features that are improved compared to the parental or reference antibody (e.g. 11C12). For instance, provided antibodies exhibit improved binding affinity for human EphA5 compared to the reference antibody (e.g. antibody with variable heavy and light chains from murine 11C12 antibody). In addition, provided antibodies also exhibit improvements in their stability as evidenced by improved thermal stability (e.g. higher Tm and Tagg compared to the reference 11C12 antibody). Conformational stability is commonly used as surrogate measurement for successful antibody stabilization as thermal stability is correlated with high expression, easier purification, longer shelf life and optimal pharmacokinetic/pharmacodynamic properties (Goswami et al. (2013) Antibodies 2:452:500; Thiagarajan et al. (2016) MAbs 8:1088-1097). Finally, among provided antibodies are antibodies that are able to bind to specifically bind cell surface EphA5 on target cells and can be internalized into the bound cell. As EphA5 is highly expressed on cancer cells, a provided antibody drug conjugates is able to recognize cancer cells that overexpress EphA5 and readily internalize the bound ADC to initiate cytotoxic effect on the cancer cell.

Thus, among provided antibodies are antibodies that exhibit one or more, or in some cases, all of the improved features of reduced immunogenicity, higher binding affinity to the EphA5 target, better thermostability and ability to be internalized by an EphA5-expressing cells. For instance, among provided antibodies that exhibit such features are the antibodies designated VH5/VK3 and VH4/VK3. In some embodiments, there also are provided antibody drug conjugates (ADCs) containing a provided antibody (e.g. VH5/VK3 or VH4/VK3) linked to a cytotoxic drug and uses thereof for treating, ameliorating, and/or preventing EphA5-expressing cancers.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### Antibodies and Antigen-Binding Fragments

Provided herein are binding polypeptides that bind to ephrin type-A receptor 5 (EphA5). For example, the binding polypeptides and antibodies specifically bind to human EPH receptor A5. Such binding polypeptides include antibodies (including antigen-binding fragments thereof) that specifically bind to EphA5, such as human EphA5. In provided embodiments, an anti-EphA5 antibody or antigen-binding fragment provided herein is a humanized antibody or an antigen-binding fragment thereof. In some embodiments, an anti-EphA5 antibody or antigen-binding fragment thereof provided herein binds human EPH receptor A5 protein. In some embodiments the human EPH receptor A5 protein comprises the amino acid set forth in SEQ ID NO: 41. In some embodiments, the binding polypeptides and antibodies of the invention bind to human EPH receptor A5 with high affinity. Preferably, the binding polypeptides and antibodies of the invention specifically recognize naturally expressed human EPH receptor A5 protein on a cell and do not cross-react to other surface molecules on that cell.

In certain aspects, the invention provides an antibody comprising an antigen-binding domain that specifically binds to an epitope of human EPH receptor A5 (EPHA5). In certain embodiments, the antigen-binding domain comprises a heavy chain variable region that comprises three heavy chain complementarity-determining regions (HCDRs) and a light chain variable region that comprises three light chain complementarity-determining regions (LCDRs).

In some embodiments, amino acid residues in variable domain sequences and full-length antibody sequences are numbered according to the Kabat numbering convention. Kabat et al. (1991), “Sequences of Proteins of Immunological Interest,” 5th Ed. Public Health Service, National Institutes of Health, Bethesda, MD. Under the Kabat numbering scheme, in some embodiments, the CDR amino acid residues in the heavy chain variable domain (VH) are numbered 31-35/35A/35B (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

However, although CDR sequences are exemplified according to the Kabat numbering convention, it will be apparent to those skilled in the art that there are alternative numbering conventions for amino acid residues in variable domain sequences and full-length antibody sequences. For instance, an alternative numbering convention is the Chothia numbering scheme, for example as set out in Chothia et al. (1989) Nature 342:877-883. Under the Chothia numbering scheme, in some embodiments, the CDR amino acids in the VH are numbered 26-32/33/34 (HCDR1), 52-56 (HCDR2), and 95-102 (HCDR3); and the CDR amino acid residues in the light chain variable domain (VL) are numbered 24-34 (LCDR1), 50-56 (LCDR2), and 89-97 (LCDR3). In some examples, amino acid residues in variable domain sequences and full-length antibody sequences are numbered according to a combination of both Chothia and Kabat CDR definitions. In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35/35A/35B (HCDR1), 50-65 (HCDR2), and 95-102 (HCDR3) in a VH, e.g., a mammalian VH, e.g., a humanized VH; and amino acid residues 24-34 (LCDR1), 50-56 (LCDR2), and 89-97 (LCDR3) in a VL, e.g., a mammalian VL, e.g., a humanized VL.

Other numbering conventions for CDR sequences available to a skilled person include, for example, “AbM” (university of Bath) and “contact” (University College London) methods. Databases can be used for CDR identification such as to identify CDRs based on Kabat numbering, Chothia or other numbering scheme. It also is understood that, in some cases, other residues are part of the CDR sequence depending on the particular convention used and factors such as the structure and protein folding of the antibody and would be understood to be so by a skilled person.

In certain aspects, the invention provides an antibody or antigen-binding fragment thereof comprising an HCDR1 comprising the amino acid sequence TFGIH (SEQ ID NO: 16) or GFTFSTFGIH (SEQ ID NO: 42). Also provided is an isolated binding polypeptide comprising an HCDR2 comprising the amino acid sequences YISGASTTIYYADTVKG (SEQ ID NO: 17), YISGASTTIYYADSVKG (SEQ ID NO: 18), or SISGASTTIYYADSVKG (SEQ ID NO: 19). Also provided is an isolated binding polypeptide comprising an HCDR3 comprising the amino acid sequence YGTSFPYGLDY (SEQ ID NO: 20). Also provided is an isolated binding polypeptide comprising a light chain variable region that comprises an LCDR1 comprising the amino acid sequences KASQSVDYDGDSYMN (SEQ ID NO: 21) or KSSQSVDYDGDSYMN (SEQ ID NO: 22). Also provided is an isolated binding polypeptide comprising an LCDR2 comprising the amino acid sequence GASNLES (SEQ ID NO: 23). Also provided is an isolated binding polypeptide comprising an LCDR3 comprising the amino acid sequence QQSNEDPFT (SEQ ID NO: 24).

In certain aspects, the invention provides an antibody comprising an HCDR1 comprising the amino acid sequence TFGIH (SEQ ID NO: 16), an HCDR2 comprising the amino acid sequence YISGASTTIYYADTVKG (SEQ ID NO: 17), an HCDR3 comprising the amino acid sequence YGTSFPYGLDY (SEQ ID NO: 20), an LCDR1 comprising the amino acid sequence KASQSVDYDGDSYMN (SEQ ID NO: 21), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence GFTFSTFGIH (SEQ ID NO: 42), an HCDR2 comprising the amino acid sequence YISGASTTIYYADTVKG (SEQ ID NO: 17), an HCDR3 comprising the amino acid sequence YGTSFPYGLDY (SEQ ID NO: 20), an LCDR1 comprising the amino acid sequence KASQSVDYDGDSYMN (SEQ ID NO: 21), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence TFGIH (SEQ ID NO: 16), an HCDR2 comprising the amino acid sequence YISGASTTIYYADSVKG (SEQ ID NO: 18), an HCDR3 comprising the amino acid sequence YGTSFPYGLDY (SEQ ID NO: 20), an LCDR1 comprising the amino acid sequence KASQSVDYDGDSYMN (SEQ ID NO: 21), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence GFTFSTFGIH (SEQ ID NO: 42), an HCDR2 comprising the amino acid sequence YISGASTTIYYADSVKG (SEQ ID NO: 18), an HCDR3 comprising the amino acid sequence YGTSFPYGLDY (SEQ ID NO: 20), an LCDR1 comprising the amino acid sequence KASQSVDYDGDSYMN (SEQ ID NO: 21), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence TFGIH (SEQ ID NO: 16), an HCDR2 comprising the amino acid sequence SISGASTTIYYADSVKG (SEQ ID NO: 19), an HCDR3 comprising the amino acid sequence YGTSFPYGLDY (SEQ ID NO: 20), an LCDR1 comprising the amino acid sequence KASQSVDYDGDSYMN (SEQ ID NO: 21), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence GFTFSTFGIH (SEQ ID NO: 42), an HCDR2 comprising the amino acid sequence SISGASTTIYYADSVKG (SEQ ID NO: 19), an HCDR3 comprising the amino acid sequence YGTSFPYGLDY (SEQ ID NO: 20), an LCDR1 comprising the amino acid sequence KASQSVDYDGDSYMN (SEQ ID NO: 21), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence TFGIH (SEQ ID NO: 16), an HCDR2 comprising the amino acid sequence YISGASTTIYYADTVKG (SEQ ID NO: 17), an HCDR3 comprising the amino acid sequence YGTSFPYGLDY (SEQ ID NO: 20), an LCDR1 comprising the amino acid sequence KSSQSVDYDGDSYMN (SEQ ID NO: 22), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence GFTFSTFGIH (SEQ ID NO: 42), an HCDR2 comprising the amino acid sequence YISGASTTIYYADTVKG (SEQ ID NO: 17), an HCDR3 comprising the amino acid sequence YGTSFPYGLDY (SEQ ID NO: 20), an LCDR1 comprising the amino acid sequence KSSQSVDYDGDSYMN (SEQ ID NO: 22), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence TFGIH (SEQ ID NO: 16), an HCDR2 comprising the amino acid sequence YISGASTTIYYADSVKG (SEQ ID NO: 18), an HCDR3 comprising the amino acid sequence YGTSFPYGLDY (SEQ ID NO: 20), an LCDR1 comprising the amino acid sequence KSSQSVDYDGDSYMN (SEQ ID NO: 22), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence GFTFSTFGIH (SEQ ID NO: 42), an HCDR2 comprising the amino acid sequence YISGASTTIYYADSVKG (SEQ ID NO: 18), an HCDR3 comprising the amino acid sequence YGTSFPYGLDY (SEQ ID NO: 20), an LCDR1 comprising the amino acid sequence KSSQSVDYDGDSYMN (SEQ ID NO: 22), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence TFGIH (SEQ ID NO: 16), an HCDR2 comprising the amino acid sequence SISGASTTIYYADSVKG (SEQ ID NO: 19), an HCDR3 comprising the amino acid sequence YGTSFPYGLDY (SEQ ID NO: 20), an LCDR1 comprising the amino acid sequence KSSQSVDYDGDSYMN (SEQ ID NO: 22), an LCDR2 comprising the amino acid sequence GASNLES (SEQ ID NO: 23), and an LCDR3 comprising the amino acid sequence QQSNEDPFT (SEQ ID NO: 24).

In certain aspects, the invention provides an antibody comprising an HCDR1 comprising the amino acid sequence GFTFSTFGIH (SEQ ID NO: 42), an HCDR2 comprising the amino acid sequence SISGASTTIYYADSVKG (SEQ ID NO: 19), an HCDR3 comprising the amino acid sequence YGTSFPYGLDY (SEQ ID NO: 20), an LCDR1 comprising the amino acid sequence KSSQSVDYDGDSYMN (SEQ ID NO: 22), an LCDR2 comprising the amino acid sequence GASNLES (SEQ ID NO: 23), and an LCDR3 comprising the amino acid sequence QQSNEDPFT (SEQ ID NO: 24).

Tolerable variations of the complementarity determining regions (CDR) sequences will be known to those of skill in the art. For example, in some embodiments the polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 16 or 42, 17, 18, 19, 20, 21, 22, 23, or 24.

In certain embodiments, the binding polypeptide, such as a provided antibody or antigen-binding fragment, comprises a heavy chain variable region comprising an amino acid sequence having at least 80%, 85%, 90%, 95%, 96%, 96%, 97%, 98%, or 99% identity to the amino acid sequence of the heavy chain variable region set forth in SEQ ID NOs: 1, 2, 3, 4, 5, 6, or 7. In certain embodiments, the binding polypeptide, such as a provided antibody or antigen-binding fragment, comprises a heavy chain variable region comprising the amino acid sequence set forth in any one of SEQ ID NOs: 1, 2, 3, 4, 5, 6, or 7. In certain embodiments, the binding polypeptide, such as a provided antibody or antigen-binding fragment, has a heavy chain variable region consisting of the amino acid sequence set forth in any one of SEQ ID NOs: 1, 2, 3, 4, 5, 6, or 7.

In certain embodiments, the binding polypeptide, such as a provided antibody or antigen-binding fragment, comprises a light chain variable region comprising an amino acid sequence having at least 80%, 85%, 90%, 95%, 96%, 97%, 98%, or 99% identity to the amino acid sequence set forth in SEQ ID NOs: 8, 9, 10, 11, 12, or 13. In certain embodiments, the binding polypeptide, such as a provided antibody or antigen-binding fragment, comprises a light chain variable region comprising the amino acid sequence set forth in any one of SEQ ID NOs: 8, 9, 10, 11, 12, or 13. In certain embodiments, the binding polypeptide, such as an antibody or antigen-binding fragment, has a light chain variable region consisting of the amino acid sequence set forth in any one of SEQ ID NOs: 8, 9, 10, 11, 12, or 13.

Among the provided binding polypeptides are humanized antibodies. In some embodiments, the binding polypeptide, such as an antibody or antigen-binding fragment thereof, comprises a heavy chain variable region comprising any of the above HCDRs and in which the amino acid has less than 100% sequence identity to SEQ ID NO: 1, and a light chain variable region comprising any of the above LCDRs and in which the amino acid sequence has less than 100% sequence identity to SEQ ID NO: 8. In some embodiments, the heavy chain variable region comprises an amino acid sequence that has at least 80% sequence identity to SEQ ID NO: 1. In some embodiments, the heavy chain variable region comprises an amino acid sequence that has at least 85% sequence identity to SEQ ID NO: 1. For instance, the heavy chain variable region comprises an amino acid sequence that has at least 86%, 87%, 87%, 88%, 89%, 90%, 91%, 92%, 93% 94%, or 95% sequence identity to SEQ ID NO: 1. In some embodiments, the light chain variable region comprises an amino acid sequence that has at least 80% sequence identity to SEQ ID NO: 8. In some embodiments, the light chain variable region comprises an amino acid sequence that has at least 85% sequence identity to SEQ ID NO: 8. For instance, the light chain variable region comprises an amino acid sequence that has at least 86%, 87%, 87%, 88%, 89%, 90%, 91%, 92%, 93% 94%, 95% sequence identity to SEQ ID NO: 8.

In certain embodiments, the binding polypeptide, such as a provided antibody or antigen-binding fragment, comprises a heavy chain variable region comprising an amino acid sequence having at least 80%, 85%, 90%, 95%, 96%, 96%, 97%, 98%, or 99% identity to the amino acid sequence of the heavy chain variable region set forth in SEQ ID NOs: 2, 3, 4, 5, 6, or 7. In certain embodiments, the binding polypeptide, such as a provided antibody or antigen-binding fragment, comprises a heavy chain variable region comprising the amino acid sequence set forth in any one of SEQ ID NOs: 2, 3, 4, 5, 6, or 7. In certain embodiments, the binding polypeptide, such as a provided antibody or antigen-binding fragment, has a heavy chain variable region consisting of the amino acid sequence set forth in any one of SEQ ID NOs: 2, 3, 4, 5, 6, or 7.

In certain embodiments, the binding polypeptide, such as a provided antibody or antigen-binding fragment, comprises a light chain variable region comprising an amino acid sequence having at least 80%, 85%, 90%, 95%, 96%, 97%, 98%, or 99% identity to the amino acid sequence set forth in SEQ ID NOs: 9, 10, 11, 12, or 13. In certain embodiments, the binding polypeptide, such as a provided antibody or antigen-binding fragment, comprises a light chain variable region comprising the amino acid sequence set forth in any one of SEQ ID NOs: 9, 10, 11, 12, or 13. In certain embodiments, the binding polypeptide, such as an antibody or antigen-binding fragment, has a light chain variable region consisting of the amino acid sequence set forth in any one of SEQ ID NOs: 9, 10, 11, 12, or 13.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 2 and a light chain variable region that has at least 80%, 85%, 90%, 95%, 96%, 96%, 97%, 98%, or 99% sequence identity to the sequence set forth in SEQ ID NO: 9. In some embodiments, the heavy chain variable region is set forth in SEQ ID NO: 2 and the light chain variable region is set forth in SEQ ID NO: 9. In some embodiments, the antibody is antibody H1K1, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 2 and a light chain variable region that has at least 80%, 85%, 90%, 95%, 96%, 96%, 97%, 98%, or 99% sequence identity to the sequence set forth in SEQ ID NO: 10. In some embodiments, the heavy chain variable region is set forth in SEQ ID NO: 2 and the light chain variable region is set forth in SEQ ID NO: 10. In some embodiments, the antibody is antibody H1K2, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 2 and a light chain variable region that has at least 80%, 85%, 90%, 95%, 96%, 96%, 97%, 98%, or 99% sequence identity to the sequence set forth in SEQ ID NO: 11. In some embodiments, the heavy chain variable region is set forth in SEQ ID NO: 2 and the light chain variable region is set forth in SEQ ID NO: 11. In some embodiments, the antibody is antibody H1K3, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 2 and a light chain variable region that has at least 80%, 85%, 90%, 95%, 96%, 96%, 97%, 98%, or 99% sequence identity to the sequence set forth in SEQ ID NO: 12. In some embodiments, the heavy chain variable region is set forth in SEQ ID NO: 2 and the light chain variable region is set forth in SEQ ID NO: 12. In some embodiments, the antibody is antibody H1K4, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 2 and a light chain variable region that has at least 80%, 85%, 90%, 95%, 96%, 96%, 97%, 98%, or 99% sequence identity to the sequence set forth in SEQ ID NO: 13. In some embodiments, the heavy chain variable region is set forth in SEQ ID NO: 2 and the light chain variable region is set forth in SEQ ID NO: 13. In some embodiments, the antibody is antibody H1K5, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 3 and a light chain variable region that has at least 80%, 85%, 90%, 95%, 96%, 96%, 97%, 98%, or 99% sequence identity to the sequence set forth in SEQ ID NO: 9. In some embodiments, the heavy chain variable region is set forth in SEQ ID NO: 3 and the light chain variable region is set forth in SEQ ID NO: 9. In some embodiments, the antibody is antibody H2K1, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 3 and a light chain variable region that has at least 80%, 85%, 90%, 95%, 96%, 96%, 97%, 98%, or 99% sequence identity to the sequence set forth in SEQ ID NO: 10. In some embodiments, the heavy chain variable region is set forth in SEQ ID NO: 3 and the light chain variable region is set forth in SEQ ID NO: 10. In some embodiments, the antibody is antibody H2K2, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 3 and a light chain variable region that has at least 80%, 85%, 90%, 95%, 96%, 96%, 97%, 98%, or 99% sequence identity to the sequence set forth in SEQ ID NO: 11. In some embodiments, the heavy chain variable region is set forth in SEQ ID NO: 3 and the light chain variable region is set forth in SEQ ID NO: 11. In some embodiments, the antibody is antibody H2K3, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 3 and a light chain variable region that has at least 80%, 85%, 90%, 95%, 96%, 96%, 97%, 98%, or 99% sequence identity to the sequence set forth in SEQ ID NO: 12. In some embodiments, the heavy chain variable region is set forth in SEQ ID NO: 3 and the light chain variable region is set forth in SEQ ID NO: 12. In some embodiments, the antibody is antibody H2K4, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 3 and a light chain variable region that has at least 80%, 85%, 90%, 95%, 96%, 96%, 97%, 98%, or 99% sequence identity to the sequence set forth in SEQ ID NO: 13. In some embodiments, the heavy chain variable region is set forth in SEQ ID NO: 3 and the light chain variable region is set forth in SEQ ID NO: 13. In some embodiments, the antibody is antibody H2K5, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 4 and a light chain variable region that has at least 80%, 85%, 90%, 95%, 96%, 96%, 97%, 98%, or 99% sequence identity to the sequence set forth in SEQ ID NO: 9. In some embodiments, the heavy chain variable region is set forth in SEQ ID NO: 4 and the light chain variable region is set forth in SEQ ID NO: 9. In some embodiments, the antibody is antibody H3K1, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 4 and a light chain variable region that has at least 80%, 85%, 90%, 95%, 96%, 96%, 97%, 98%, or 99% sequence identity to the sequence set forth in SEQ ID NO: 10. In some embodiments, the heavy chain variable region is set forth in SEQ ID NO: 4 and the light chain variable region is set forth in SEQ ID NO: 10. In some embodiments, the antibody is antibody H3K2, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 4 and a light chain variable region that has at least 80%, 85%, 90%, 95%, 96%, 96%, 97%, 98%, or 99% sequence identity to the sequence set forth in SEQ ID NO: 11. In some embodiments, the heavy chain variable region is set forth in SEQ ID NO: 4 and the light chain variable region is set forth in SEQ ID NO: 11. In some embodiments, the antibody is antibody H3K3, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 4 and a light chain variable region that has at least 80%, 85%, 90%, 95%, 96%, 96%, 97%, 98%, or 99% sequence identity to the sequence set forth in SEQ ID NO: 12. In some embodiments, the heavy chain variable region is set forth in SEQ ID NO: 4 and the light chain variable region is set forth in SEQ ID NO: 12. In some embodiments, the antibody is antibody H3K4, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 4 and a light chain variable region that has at least 80%, 85%, 90%, 95%, 96%, 96%, 97%, 98%, or 99% sequence identity to the sequence set forth in SEQ ID NO: 13. In some embodiments, the heavy chain variable region is set forth in SEQ ID NO: 4 and the light chain variable region is set forth in SEQ ID NO: 13. In some embodiments, the antibody is antibody H3K5, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 5 and a light chain variable region that has at least 80%, 85%, 90%, 95%, 96%, 96%, 97%, 98%, or 99% sequence identity to the sequence set forth in SEQ ID NO: 9. In some embodiments, the heavy chain variable region is set forth in SEQ ID NO: 5 and the light chain variable region is set forth in SEQ ID NO: 9. In some embodiments, the antibody is antibody H4K1, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 5 and a light chain variable region that has at least 80%, 85%, 90%, 95%, 96%, 96%, 97%, 98%, or 99% sequence identity to the sequence set forth in SEQ ID NO: 10. In some embodiments, the heavy chain variable region is set forth in SEQ ID NO: 5 and the light chain variable region is set forth in SEQ ID NO: 10. In some embodiments, the antibody is antibody H4K2, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 5 and a light chain variable region that has at least 80%, 85%, 90%, 95%, 96%, 96%, 97%, 98%, or 99% sequence identity to the sequence set forth in SEQ ID NO: 11. In some embodiments, the heavy chain variable region is set forth in SEQ ID NO: 5 and the light chain variable region is set forth in SEQ ID NO: 11. In some embodiments, the antibody is antibody H4K3, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 5 and a light chain variable region that has at least 80%, 85%, 90%, 95%, 96%, 96%, 97%, 98%, or 99% sequence identity to the sequence set forth in SEQ ID NO: 12. In some embodiments, the heavy chain variable region is set forth in SEQ ID NO: 5 and the light chain variable region is set forth in SEQ ID NO: 12. In some embodiments, the antibody is antibody H4K4, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 5 and a light chain variable region that has at least 80%, 85%, 90%, 95%, 96%, 96%, 97%, 98%, or 99% sequence identity to the sequence set forth in SEQ ID NO: 13. In some embodiments, the heavy chain variable region is set forth in SEQ ID NO: 5 and the light chain variable region is set forth in SEQ ID NO: 13. In some embodiments, the antibody is antibody H4K5, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 6 and a light chain variable region that has at least 80%, 85%, 90%, 95%, 96%, 96%, 97%, 98%, or 99% sequence identity to the sequence set forth in SEQ ID NO: 9. In some embodiments, the heavy chain variable region is set forth in SEQ ID NO: 6 and the light chain variable region is set forth in SEQ ID NO: 9. In some embodiments, the antibody is antibody H5K1, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 6 and a light chain variable region that has at least 80%, 85%, 90%, 95%, 96%, 96%, 97%, 98%, or 99% sequence identity to the sequence set forth in SEQ ID NO: 10. In some embodiments, the heavy chain variable region is set forth in SEQ ID NO: 6 and the light chain variable region is set forth in SEQ ID NO: 10. In some embodiments, the antibody is antibody H5K2, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 6 and a light chain variable region that has at least 80%, 85%, 90%, 95%, 96%, 96%, 97%, 98%, or 99% sequence identity to the sequence set forth in SEQ ID NO: 11. In some embodiments, the heavy chain variable region is set forth in SEQ ID NO: 6 and the light chain variable region is set forth in SEQ ID NO: 11. In some embodiments, the antibody is antibody H5K3, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 6 and a light chain variable region that has at least 80%, 85%, 90%, 95%, 96%, 96%, 97%, 98%, or 99% sequence identity to the sequence set forth in SEQ ID NO: 12. In some embodiments, the heavy chain variable region is set forth in SEQ ID NO: 6 and the light chain variable region is set forth in SEQ ID NO: 12. In some embodiments, the antibody is antibody H5K4, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 6 and a light chain variable region that has at least 80%, 85%, 90%, 95%, 96%, 96%, 97%, 98%, or 99% sequence identity to the sequence set forth in SEQ ID NO: 13. In some embodiments, the heavy chain variable region is set forth in SEQ ID NO: 6 and the light chain variable region is set forth in SEQ ID NO: 13. In some embodiments, the antibody is antibody H5K5, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 7 and a light chain variable region that has at least 80%, 85%, 90%, 95%, 96%, 96%, 97%, 98%, or 99% sequence identity to the sequence set forth in SEQ ID NO: 9. In some embodiments, the heavy chain variable region is set forth in SEQ ID NO: 7 and the light chain variable region is set forth in SEQ ID NO: 9. In some embodiments, the antibody is antibody H6K1, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 7 and a light chain variable region that has at least 80%, 85%, 90%, 95%, 96%, 96%, 97%, 98%, or 99% sequence identity to the sequence set forth in SEQ ID NO: 10. In some embodiments, the heavy chain variable region is set forth in SEQ ID NO: 7 and the light chain variable region is set forth in SEQ ID NO: 10. In some embodiments, the antibody is antibody H6K2, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 7 and a light chain variable region that has at least 80%, 85%, 90%, 95%, 96%, 96%, 97%, 98%, or 99% sequence identity to the sequence set forth in SEQ ID NO: 11. In some embodiments, the heavy chain variable region is set forth in SEQ ID NO: 7 and the light chain variable region is set forth in SEQ ID NO: 11. In some embodiments, the antibody is antibody H6K3, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 7 and a light chain variable region that has at least 80%, 85%, 90%, 95%, 96%, 96%, 97%, 98%, or 99% sequence identity to the sequence set forth in SEQ ID NO: 12. In some embodiments, the heavy chain variable region is set forth in SEQ ID NO: 7 and the light chain variable region is set forth in SEQ ID NO: 12. In some embodiments, the antibody is antibody H6K4, such as a full-length humanized antibody or an antigen-binding fragment thereof.

In some embodiments, the binding polypeptide, such as an anti-EphA5 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 7 and a light chain variable region that has at least 80%, 85%, 90%, 95%, 96%, 96%, 97%, 98%, or 99% sequence identity to the sequence set forth in SEQ ID NO: 13. In some embodiments, the heavy chain variable region is set forth in SEQ ID NO: 7 and the light chain variable region is set forth in SEQ ID NO: 13. In some embodiments, the antibody is antibody H6K5, such as a full-length humanized antibody or an antigen-binding fragment thereof.

Provided is an antibody comprising a heavy chain variable region comprising the amino acid sequence set forth in SEQ ID NO: 5 and a light chain variable region comprising the amino acid sequence set forth in SEQ ID NO: 11 (i.e. H4K3).

Provided is an antibody comprising a heavy chain variable region comprising the amino acid sequence set forth in SEQ ID NO: 6 and a light chain variable region comprising the amino acid sequence set forth in SEQ ID NO: 11 (i.e. H5K3).

An antibody of the invention can be prepared using an antibody having one or more of the VH and/or VL sequences disclosed herein as a starting material to engineer a modified antibody, which modified antibody may have altered properties as compared with the starting antibody. An antibody can be engineered by modifying one or more amino acids within one or both variable regions (i.e., VH and/or VL), for example within one or more CDR regions and/or within one or more framework regions. Additionally, or alternatively, an antibody can be engineered by modifying residues within the constant region(s), for example to alter the effector function(s) of the antibody.

In some embodiments, the binding polypeptide is an antibody or an antigen-binding fragment thereof.

Among the provided antibodies are antibody fragments. Examples of antibody fragments include but are not limited to Fv, Fab, Fab′, Fab′-SH, F(ab′)₂; diabodies; linear antibodies; heavy chain variable (VH) regions, single-chain antibody molecules such as scFvs and single-domain antibodies comprising only the VH region. In some embodiments, the antigen-binding fragment is selected from the group consisting of a Fab, a single-chain variable fragment (scFv), a single-domain antibody, and a nanobody. In further embodiments, the antibody is a full-length antibody. In provided embodiments, the antibody or antigen-binding fragment is a humanized antibody or an antigen-binding fragment thereof.

In some embodiments, the antibody or antigen-binding fragment thereof may contain at least a portion of an immunoglobulin constant region, such as one or more constant region domain. In some embodiments, the constant region of the heavy chain includes at least the heavy chain constant region 1 (CH1) and the constant region of the light chain includes the light chain constant region. In some embodiments, the constant region of the heavy chain further includes a hinge domain, a CH2 and/or CH3 domain. In some embodiments, the heavy chain constant domain is from a constant chain, or a portion thereof containing the CH1, hinge, CH2 and/or CH3, of a human IgG, such as a human IgG1 or IgG4.

In some embodiments, the light chain constant domain is from a constant chain of a human kappa light or lambda light chain.

In some embodiments, the light chain constant domain is from a constant chain of a human kappa light chain.

In some embodiments, the light chain variable domain is from a variable chain of a human kappa light or lambda light chain.

In some embodiments, the light chain variable domain is from a variable chain of a human kappa light chain.

Notation herein referring to VK or Vκ is in connection with the variable portion of the kappa light chain(s).

Among the provided antibodies are full-length antibodies containing a heavy chain with any one of the heavy chain variable regions provided herein combined with a human heavy chain constant region; and a light chain with any one of the light chain variable regions provided herein combined with a human light chain constant region. In some embodiments, the constant region of the heavy chain is a human IgG1 heavy chain constant region. In some embodiments, the heavy chain constant region is set forth in SEQ ID NO: 14 or a sequence that has at least 85%, 86%, 87%, 88%, 89%, 90%, 91%, 92%, 93%, 94%, 95%, 96%, 97%, 98%, or 99% sequence identity to SEQ ID NO: 14. In some embodiments, the heavy chain constant region is set forth in SEQ ID NO: 14. In some embodiments, the constant region of the light chain is a human kappa light chain constant region. In some embodiments, the light chain constant region is set forth in SEQ ID NO: 15 or a sequence that has at least 85%, 86%, 87%, 88%, 89%, 90%, 91%, 92%, 93%, 94%, 95%, 96%, 97%, 98%, or 99% sequence identity to SEQ ID NO: 15. In some embodiments, the heavy chain constant region is set forth in SEQ ID NO: 15. Any of the heavy chain variable regions provided herein may be combined with a suitable human constant region. Any of the light chain variable regions may be combined with a suitable human light chain constant region.

In some embodiments, provided herein is a full-length antibody (e.g. chimeric antibody) comprising a heavy chain comprising a heavy chain variable domain comprising the amino acid sequence set forth in SEQ ID NO: 1 and a heavy chain constant domain comprising the amino acid sequence set forth in SEQ ID NO: 14, and a light chain comprising a light chain variable domain comprising an amino acid sequence set forth in SEQ ID NO: 8 and a light chain constant domain comprising the amino acid sequence set forth in SEQ ID NO: 15. In some embodiments, the full-length antibody is composed of a heavy chain comprising a heavy chain variable domain consisting of the amino acid sequence set forth in SEQ ID NO: 1 and a heavy chain constant domain consisting of the amino acid sequence set forth in SEQ ID NO: 14, and a light chain comprising a light chain variable domain consisting of the amino acid sequence set forth in any one of SEQ ID NO: 8 and a light chain constant domain consisting of the amino acid sequence set forth in SEQ ID NO: 15.

In some embodiments, provided herein is a full-length antibody (e.g. humanized antibody) comprising a heavy chain comprising a heavy chain variable domain comprising an amino acid sequence set forth in SEQ ID NOs: 2, 3, 4, 5, 6, or 7 and a heavy chain constant domain comprising the amino acid sequence set forth in SEQ ID NO: 14, and a light chain comprising a light chain variable domain comprising an amino acid sequence set forth in SEQ ID NOs: 9, 10, 11, 12, or 13 and a light chain constant domain comprising the amino acid sequence set forth in SEQ ID NO: 15.

In some embodiments, the full-length antibody is composed of a heavy chain comprising a heavy chain variable domain consisting of the amino acid sequence set forth in any one of SEQ ID NOs: 2, 3, 4, 5, 6, or 7 and a heavy chain constant domain consisting of the amino acid sequence set forth in SEQ ID NO: 14, and a light chain comprising a light chain variable domain consisting of the amino acid sequence set forth in any one of SEQ ID NOs: 9, 10, 11, 12, or 13 and a light chain constant domain consisting of the amino acid sequence set forth in SEQ ID NO: 15.

In some embodiments, provided herein is a full-length antibody comprising a heavy chain comprising the amino acid sequence set forth in SEQ ID NOs: 38 or 39 and a light chain comprising the amino acid sequence set forth in SEQ ID NO: 40.

In some embodiments, provided herein is a full-length antibody comprising a full-length heavy chain consisting of the amino acid sequence set forth in SEQ ID NOs: 38 or 39 and a full-length light chain consisting of the amino acid sequence set forth in SEQ ID NO: 40.

Tolerable variations of the full-length antibody sequences will be known to those of skill in the art. For example, in some embodiments the antibody comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 38, 39, and 40.

In some embodiments, the provided anti-EpHA5 antibody is a full-length antibody composed of (i) a heavy chain comprising at least 85%, 90%, 91%, 92%, 93%, 94%, 95%, 96%, 97%, 98%, or 99% sequence identity to the amino acid sequence set forth in SEQ ID NO: 38; and (ii) a light chain comprising at least at or about 85%, 90%, 91%, 92%, 93%, 94%, 95%, 96%, 97%, 98%, or 99% sequence identity to the amino acid sequence set forth in SEQ ID NO: 40. In some embodiments, the variable region of the heavy chain comprises a HCDR1 comprising the amino acid sequence set forth in SEQ ID NO: 16, a HCDR2 comprising the amino acid sequence set forth in SEQ ID NO: 17, and a HCDR3 comprising the amino acid sequence set forth in SEQ ID NO: 20; and the variable region of the light chain comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

Provided is an anti-EphA5 antibody comprising a full-length heavy chain comprising the amino acid sequence set forth in SEQ ID NO: 38 and a full-length light chain comprising the amino acid sequence set forth in SEQ ID NO: 40 (i.e. H4K3).

In some embodiments, the provided anti-EphA5 antibody is a full-length antibody composed of (i) a heavy chain comprising at least 85%, 90%, 91%, 92%, 93%, 94%, 95%, 96%, 97%, 98%, or 99% sequence identity to the amino acid sequence set forth in SEQ ID NO: 39; and (ii) a light chain comprising at least 85%, 90%, 91%, 92%, 93%, 94%, 95%, 96%, 97%, 98%, or 99% sequence identity to the amino acid sequence set forth in SEQ ID NO: 40. In some embodiments, the variable region of the heavy chain comprises a HCDR1 comprising the amino acid sequence set forth in SEQ ID NO: 16, a HCDR2 comprising the amino acid sequence set forth in SEQ ID NO: 18, and a HCDR3 comprising the amino acid sequence set forth in SEQ ID NO: 20; and the variable region of the light chain comprises a LCDR1 comprising the amino acid sequence set forth in SEQ ID NO: 21, a LCDR2 comprising the amino acid sequence set forth in SEQ ID NO: 23, and a LCDR3 comprising the amino acid sequence set forth in SEQ ID NO: 24.

Also provided is an anti-EpHA5 antibody comprising a full-length heavy chain comprising the amino acid sequence set forth in SEQ ID NO: 39 and a full-length light chain comprising the amino acid sequence set forth in SEQ ID NO: 40 (i.e. H5K3).

In certain embodiments, the invention includes an antibody that binds to the same epitope on human EPH receptor A5 as an antibody of the invention (i.e., antibodies that have the ability to cross-compete for binding to human EPH receptor A5 with any of the antibodies of the invention). In a preferred embodiment, the reference antibody or antibody fragment for cross-competition studies can be one of the antibodies or antibody fragments described herein, as comparisons are made using equivalent molecules (e.g. a Fab to a Fab, full-length antibody to a full-length antibody, etc.). For example, surface plasmon resonance, or “SPR” analysis, ELISA assays or flow cytometry may be used to demonstrate cross-competition with the antibodies of the current invention. The ability of a test antibody to inhibit the binding of, for example H4K3 or H5K3, to human EPH receptor A5 demonstrates that the test antibody can compete with H4K3 or H5K3 for binding to human EPH receptor A5 and thus is considered to bind to the same epitope of human EPH receptor A5 as H4K3 or H5K3.

In some embodiments, any one of the provided anti-EphA5 antibodies are isolated, purified or semi-purified such that they retain specificity in the desired application. Proteins may be recombinant, or synthesized in vitro, although in most embodiments, the proteins are recombinant. Alternatively, a non-recombinant or recombinant protein may be isolated from bacteria.

TABLE 1
Sequences used in the invention

| SEQ | | | |
|---|---|---|---|
| ID | | | |
| NO: | Name | Type | Sequence |
| 1 | Chimeric | PRT | EVTLKESGGGLVQPGGSRKLSCAASGFTFSTFGIHWV |
| | VH0 | | RQAPEKGLEWVAYISGASTTIYYADTVKGRFTISRDNP |
| | Variable | | KNTLFLQMTSLRSEDTAMYYCARYGTSFPYGLDYWG |
| | Heavy Chain | | QGTSVTVSS |
| 2 | VH1 | PRT | EVTLVESGGGLVQPGGSRKLSCAASGFTFSTFGIHWV |
| | Variable | | RQAPGKGLEWVAYISGASTTIYYADTVKGRFTISRDNS |
| | Heavy Chain | | KNTLYLQMNSLRSEDTAMYYCARYGTSFPYGLDYWG |
| | | | QGTSVTVSS |
| 3 | VH2 | PRT | EVTLVESGGGLVQPGGSLKLSCAASGFTFSTFGIHWVR |
| | Variable | | QAPGKGLEWVAYISGASTTIYYADTVKGRFTISRDNS |
| | Heavy Chain | | KNTLYLQMNSLRSEDTAMYYCARYGTSFPYGLDYWG |
| | | | QGTLVTVSS |
| 4 | VH3 | PRT | EVQLVESGGGLVQPGGSLKLSCAASGFTFSTFGIHWV |
| | Variable | | RQAPGKGLEWVAYISGASTTIYYADTVKGRFTISRDNS |
| | Heavy Chain | | KNTLYLQMNSLRSEDTAMYYCARYGTSFPYGLDYWG |
| | | | QGTLVTVSS |
| 5 | VH4 | PRT | EVQLVESGGGLVQPGGSLKLSCAASGFTFSTFGIHWV |
| | Variable | | RQAPGKGLEWVAYISGASTTIYYADTVKGRFTISRDNS |
| | Heavy Chain | | KNTLYLQMNSLRAEDTAVYYCARYGTSFPYGLDYWG |
| | | | QGTLVTVSS |
| 6 | VH5 | PRT | EVQLVESGGGLVQPGGSLKLSCAASGFTFSTFGIHWV |
| | Variable | | RQAPGKGLEWVAYISGASTTIYYADSVKGRFTISRDNS |
| | Heavy Chain | | KNTLYLQMNSLRAEDTAVYYCARYGTSFPYGLDYWG |
| | | | QGTLVTVSS |
| 7 | VH6 | PRT | EVQLVESGGGLVQPGGSLKLSCAASGFTFSTFGIHWV |
| | Variable | | RQAPGKGLEWVASISGASTTIYYADSVKGRFTISRDNS |
| | Heavy Chain | | KNTLYLQMNSLRAEDTAVYYCARYGTSFPYGLDYWG |
| | | | QGTLVTVSS |
| 8 | Chimeric | PRT | DIVMTQSPGSLAVSLGQRATISCKASQSVDYDGDSYM |
| | VK0 | | NWYQQKPGQPPKLLIYGASNLESGIPARFSGSGSGTDF |
| | Variable | | TLNIHPVEEEDAASYYCQQSNEDPFTFGSGTKLEIK |
| | Light Chain | | |
| 9 | VK1 | PRT | DIVMTQSPGSLAVSLGERATINCKASQSVDYDGDSYM |
| | Variable | | NWYQQKPGKAPKLLIYGASNLESGIPDRFSGSGSGTDF |
| | Light Chain | | TLTISRLEEEDAASYYCQQSNEDPFTFGQGTKLEIK |
| 10 | VK2 | PRT | DIVMTQSPDSLAVSLGERATINCKASQSVDYDGDSYM |
| | Variable | | NWYQQKPGKAPKLLIYGASNLESGIPDRESGSGSGTDF |
| | Light Chain | | TLTISRLEPEDAASYYCQQSNEDPFTFGQGTKLEIK |
| 11 | VK3 | PRT | DIVMTQSPDSLAVSLGERATINCKASQSVDYDGDSYM |
| | Variable | | NWYQQKPGKAPKLLIYGASNLESGIPDRFSGSGSGTDF |
| | Light Chain | | TLTISRLEPEDAAVYYCQQSNEDPFTFGQGTKLEIK |
| 12 | VK4 | PRT | DIVMTQSPDSLAVSLGERATINCKSSQSVDYDGDSYM |
| | Variable | | NWYQQKPGKAPKLLIYGASNLESGIPDRFSGSGSGTDF |
| | Light Chain | | TLTISRLEPEDAASYYCQQSNEDPFTFGQGTKLEIK |
| 13 | VK5 | PRT | DIVMTQSPDSLAVSLGERATINCKSSQSVDYDGDSYM |
| | Variable | | NWYQQKPGKAPKLLIYGASNLESGIPDRESGSGSGTDF |
| | Light Chain | | TLTISRLEPEDAAVYYCQQSNEDPFTFGQGTKLEIK |
| 14 | Heavy Chain | PRT | ASTKGPSVFPLAPSSKSTSGGTAALGCLVKDYFPEPVT |
| | Constant | | VSWNSGALTSGVHTFPAVLQSSGLYSLSSVVTVPSSSL |
| | Domain | | GTQTYICNVNHKPSNTKVDKRVEPKSCDKTHTCPPCP |
| | | | APELLGGPSVFLFPPKPKDTLMISRTPEVTCVVVDVSH |
| | | | EDPEVKFNWYVDGVEVHNAKTKPREEQYNSTYRVVS |
| | | | VLTVLHQDWLNGKEYKCKVSNKALPAPIEKTISKAKG |
| | | | QPREPQVYTLPPSREEMTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| 15 | Light Chain | PRT | RTVAAPSVFIFPPSDEQLKSGTASVVCLLNNFYPREAK |
| | Constant | | VQWKVDNALQSGNSQESVTEQDSKDSTYSLSSTLTLS |
| | Domain | | KADYEKHKVYACEVTHQGLSSPVTKSFNRGEC |
| 16 | VH0-6 | PRT | TFGIH |
| | CDR1 | | |
| 17 | VH0-4 | PRT | YISGASTTIYYADTVKG |
| | CDR2 | | |
| 18 | VH5 CDR2 | PRT | YISGASTTIYYADSVKG |
| 19 | VH6 CDR2 | PRT | SISGASTTIYYADSVKG |
| 20 | VH0-6 | PRT | YGTSFPYGLDY |
| | CDR3 | | |
| 21 | VK0-3 | PRT | KASQSVDYDGDSYMN |
| | CDR1 | | |
| 22 | VK4-5 | PRT | KSSQSVDYDGDSYMN |
| | CDR1 | | |
| 23 | VK0-5 | PRT | GASNLES |
| | CDR2 | | |
| 24 | VK0-5 | PRT | QQSNEDPFT |
| | CDR3 | | |
| 25 | VH0 | DNA | GAAGTGACCCTGAAAGAGTCTGGCGGAGGACTGGT |
| | Variable | | TCAGCCTGGCGGATCTAGAAAGCTGTCTTGTGCCGC |
| | heavy chain | | CTCCGGCTTCACCTTCTCTACCTTTGGCATCCACTGG |
| | | | GTCCGACAGGCCCCTGAGAAAGGATTGGAGTGGGT |
| | | | CGCCTACATCTCCGGCGCTTCCACCACCATCTACTA |
| | | | CGCCGATACCGTGAAGGGCAGATTCACCATCAGCC |
| | | | GGGACAACCCCAAGAACACCCTGTTTCTGCAGATG |
| | | | ACCAGCCTGCGGAGCGAGGACACCGCCATGTACTA |
| | | | CTGTGCCAGATACGGCACCAGCTTTCCCTACGGCCT |
| | | | GGATTATTGGGGCCAGGGAACCTCCGTTACAGTCTC |
| | | | CTCA |
| 26 | VH1 | DNA | GAAGTGACCCTGGTTGAATCTGGCGGAGGACTGGTT |
| | Variable | | CAGCCTGGCGGCTCTAGAAAGCTGTCTTGTGCCGCC |
| | heavy chain | | TCTGGCTTCACCTTCTCCACCTTTGGCATCCACTGG |
| | | | GTCCGACAGGCCCCTGGCAAAGGATTGGAGTGGGTC |
| | | | GCCTATATCTCCGGCGCCTCCACCACCATCTACTAC |
| | | | GCCGATACCGTGAAGGGCAGATTCACCATCAGCCG |
| | | | GGACAACTCCAAGAACACCCTGTACCTGCAGATGA |
| | | | ACTCCCTGCGGAGCGAGGACACCGCCATGTACTACT |
| | | | GTGCCAGATACGGCACCAGCTTTCCCTACGGCCTGG |
| | | | ATTATTGGGGCCAGGGAACCTCCGTTACAGTCTCCT |
| | | | CA |
| 27 | VH2 | DNA | GAAGTGACCCTGGTTGAATCTGGCGGAGGACTGGTT |
| | Variable | | CAGCCTGGCGGATCTCTGAAGCTGTCTTGTGCCGCC |
| | heavy chain | | TCTGGCTTCACCTTCTCCACCTTTGGCATCCACTGG |
| | | | GTCCGACAGGCCCCTGGCAAAGGATTGGAGTGGGTC |
| | | | GCCTATATCTCCGGCGCCTCCACCACCATCTACTAC |
| | | | GCCGATACCGTGAAGGGCAGATTCACCATCAGCCG |
| | | | GGACAACTCCAAGAACACCCTGTACCTGCAGATGA |
| | | | ACTCCCTGCGGAGCGAGGACACCGCCATGTACTACT |
| | | | GTGCCAGATACGGCACCAGCTTTCCCTACGGCCTGG |
| | | | ATTATTGGGGCCAGGGAACCCTGGTTACAGTCTCCT |
| | | | CA |
| 28 | VH3 | DNA | GAAGTGCAGCTGGTTGAATCTGGCGGAGGACTGGT |
| | Variable | | TCAGCCTGGCGGATCTCTGAAGCTGTCTTGTGCCGC |
| | heavy chain | | CTCTGGCTTCACCTTCTCCACCTTTGGCATCCACTG |
| | | | GGTCCGACAGGCCCCTGGCAAAGGATTGGAGTGGGT |
| | | | CGCCTATATCTCCGGCGCCTCCACCACCATCTACTA |
| | | | CGCCGATACCGTGAAGGGCAGATTCACCATCAGCC |
| | | | GGGACAACTCCAAGAACACCCTGTACCTGCAGATG |
| | | | AACTCCCTGCGGAGCGAGGACACCGCCATGTACTA |
| | | | CTGTGCCAGATACGGCACCAGCTTTCCCTACGGCCT |
| | | | GGATTATTGGGGCCAGGGAACCCTGGTTACAGTCTC |
| | | | CTCA |
| 29 | VH4 | DNA | GAAGTGCAGCTGGTTGAATCTGGCGGAGGACTGGT |
| | Variable | | TCAGCCTGGCGGATCTCTGAAGCTGTCTTGTGCCGC |
| | heavy chain | | CTCTGGCTTCACCTTCTCCACCTTTGGCATCCACTGG |
| | | | GTCCGACAGGCCCCTGGCAAAGGATTGGAGTGGGT |
| | | | CGCCTATATCTCCGGCGCCTCCACCACCATCTACTA |
| | | | CGCCGATACCGTGAAGGGCAGATTCACCATCAGCC |
| | | | GGGACAACTCCAAGAACACCCTGTACCTGCAGATG |
| | | | AACTCCCTGAGAGCCGAGGACACCGCCGTGTACTA |
| | | | CTGTGCCAGATACGGCACCAGCTTTCCCTACGGCCT |
| | | | GGATTATTGGGGCCAGGGAACCCTGGTTACAGTCTC |
| | | | CTCA |
| 30 | VH5 | DNA | GAAGTGCAGCTGGTTGAATCTGGCGGAGGACTGGT |
| | Variable | | TCAGCCTGGCGGATCTCTGAAGCTGTCTTGTGCCGC |
| | heavy chain | | CTCTGGCTTCACCTTCTCCACCTTTGGCATCCACTGG |
| | | | GTCCGACAGGCCCCTGGCAAAGGATTGGAGTGGGT |
| | | | CGCCTATATCTCCGGCGCCTCCACCACCATCTACTA |
| | | | CGCCGATTCTGTGAAGGGCAGATTCACCATCAGCCG |
| | | | GGACAACTCCAAGAACACCCTGTACCTGCAGATGA |
| | | | ACTCCCTGAGAGCCGAGGACACCGCCGTGTACTACT |
| | | | GTGCCAGATACGGCACCAGCTTTCCCTACGGCCTGG |
| | | | ATTATTGGGGCCAGGGAACCCTGGTTACAGTCTCCT |
| | | | CA |
| 31 | VH6 | DNA | GAAGTGCAGCTGGTTGAATCTGGCGGAGGACTGGT |
| | Variable | | TCAGCCTGGCGGATCTCTGAAGCTGTCTTGTGCCGC |
| | heavy chain | | CTCTGGCTTCACCTTCTCCACCTTTGGCATCCACTGG |
| | | | GTCCGACAGGCCCCTGGCAAAGGATTGGAATGGGT |
| | | | CGCCTCTATCTCCGGCGCCTCCACCACCATCTACTA |
| | | | CGCCGATTCTGTGAAGGGCAGATTCACCATCAGCCG |
| | | | GGACAACTCCAAGAACACCCTGTACCTGCAGATGA |
| | | | ACTCCCTGAGAGCCGAGGACACCGCCGTGTACTACT |
| | | | GTGCCAGATACGGCACCAGCTTTCCCTACGGCCTGG |
| | | | ATTATTGGGGCCAGGGAACCCTGGTTACAGTCTCCT |
| | | | CA |
| 32 | VK0 | DNA | GACATCGTGATGACACAGTCTCCAGGCAGCCTGGCT |
| | Variable light | | GTGTCCTTGGGACAGAGAGCTACCATCTCCTGCAAG |
| | chain | | GCCTCTCAGTCCGTGGACTACGACGGCGACTCCTAC |
| | | | ATGAACTGGTATCAGCAGAAGCCCGGCCAGCCTCCT |
| | | | AAGCTGTTGATCTACGGCGCCTCCAACCTGGAAAGC |
| | | | GGCATCCCTGCTAGATTCTC |
| | | | CGGCTCTGGCTCTGGCACCGACTTCACCCTGAACAT |
| | | | CCATCCTGTGGAAGAAGAGGACGCCGCCTCCTACTA |
| | | | CTGCCAGCAGTCTAACGAGGACCCCTTCACCTTCGG |
| | | | CTCCGGCACAAAATTGGAGATCAAA |
| 33 | VK1 | DNA | GACATCGTGATGACACAGTCTCCAGGCAGCCTGGCT |
| | Variable light | | GTGTCTCTGGGAGAGAGAGCCACCATCAACTGCAA |
| | chain | | GGCCTCTCAGTCCGTGGACTACGACGGCGACTCCTA |
| | | | CATGAACTGGTATCAGCAGAAGCCCGGCAAGGCCC |
| | | | CTAAGCTGTTGATCTACGGCGCCTCCAACCTGGAAA |
| | | | GCGGCATCCCTGATAGATTCTCCGGCTCTGGCTCTG |
| | | | GCACCGACTTCACCCTGACCATCTCTCGGCTGGAAG |
| | | | AGGAAGATGCCGCCTCCTACTACTGCCAGCAGTCCA |
| | | | ACGAGGACCCCTTCACCTTTGGCCAGGGCACAAAGT |
| | | | TGGAGATCAAA |
| 34 | VK2 | DNA | GACATCGTGATGACCCAGTCTCCAGACAGCCTGGCT |
| | Variable light | | GTGTCTCTGGGCGAGAGAGCCACCATCAACTGCAA |
| | chain | | GGCCTCTCAGTCCGTGGACTACGACGGCGACTCCTA |
| | | | CATGAACTGGTATCAGCAGAAGCCCGGCAAGGCCC |
| | | | CTAAGCTGTTGATCTACGGCGCCTCCAACCTGGAAA |
| | | | GCGGCATCCCTGATAGATTCTCCGGCTCTGGCTCTG |
| | | | GCACCGACTTCACCCTGACCATCTCCAGACTGGAAC |
| | | | CTGAGGACGCCGCCTCCTACTACTGCCAGCAGTCTA |
| | | | ACGAGGACCCCTTCACCTTTGGCCAGGGCACAAAGT |
| | | | TGGAGATCAAA |
| 35 | VK3 | DNA | GACATCGTGATGACCCAGTCTCCAGACAGCCTGGCT |
| | Variable light | | GTGTCTCTGGGCGAGAGAGCCACCATCAACTGCAA |
| | chain | | GGCCTCTCAGTCCGTGGACTACGACGGCGACTCCTA |
| | | | CATGAACTGGTATCAGCAGAAGCCCGGCAAGGCCC |
| | | | CTAAGCTGTTGATCTACGGCGCCTCCAACCTGGAAA |
| | | | GCGGCATCCCTGATAGATTCTCCGGCTCTGGCTCTG |
| | | | GCACCGACTTCACCCTGACCATCTCCAGACTGGAAC |
| | | | CTGAGGATGCCGCCGTGTACTACTGCCAGCAGTCTA |
| | | | ACGAGGACCCCTTCACCTTTGGCCAGGGCACAAAGT |
| | | | TGGAGATCAAA |
| 36 | VK4 | DNA | GACATCGTGATGACCCAGTCTCCAGACAGCCTGGCT |
| | Variable light | | GTGTCTCTGGGCGAGAGAGCCACCATCAACTGCAA |
| | chain | | GTCCTCTCAGTCCGTGGACTACGACGGCGACTCCTA |
| | | | CATGAACTGGTATCAGCAGAAGCCCGGCAAGGCCC |
| | | | CTAAGCTGTTGATCTACGGCGCCTCCAACCTGGAAA |
| | | | GCGGCATCCCTGATAGATTCTCCGGCTCTGGCTCTG |
| | | | GCACCGACTTCACCCTGACCATCTCCAGACTGGAAC |
| | | | CTGAGGACGCCGCCTCCTACTACTGCCAGCAGTCTA |
| | | | ACGAGGACCCCTTCACCTTTGGCCAGGGCACAAAGT |
| | | | TGGAGATCAAA |
| 37 | VK5 | DNA | GACATCGTGATGACCCAGTCTCCAGACAGCCTGGCT |
| | Variable light | | GTGTCTCTGGGCGAGAGAGCCACCATCAACTGCAA |
| | chain | | GTCCTCTCAGTCCGTGGACTACGACGGCGACTCCTA |
| | | | CATGAACTGGTATCAGCAGAAGCCCGGCAAGGCCC |
| | | | CTAAGCTGTTGATCTACGGCGCCTCCAACCTGGAAA |
| | | | GCGGCATCCCTGATAGATTCTCCGGCTCTGGCTCTG |
| | | | GCACCGACTTCACCCTGACCATCTCCAGACTGGAAC |
| | | | CTGAGGATGCCGCCGTGTACTACTGCCAGCAGTCTA |
| | | | ACGAGGACCCCTTCACCTTTGGCCAGGGCACAAAGT |
| | | | TGGAGATCAAA |
| 38 | VH4 Full- | PRT | EVQLVESGGGLVQPGGSLKLSCAASGFTFSTFGIHWV |
| | length heavy | | RQAPGKGLEWVAYISGASTTIYYADTVKGRFTISRDNS |
| | chain | | KNTLYLQMNSLRAEDTAVYYCARYGTSFPYGLDYWG |
| | | | QGTLVTVSSASTKGPSVFPLAPSSKSTSGGTAALGCLV |
| | | | KDYFPEPVTVSWNSGALTSGVHTFPAVLQSSGLYSLSS |
| | | | VVTVPSSSLGTQTYICNVNHKPSNTKVDKRVEPKSCD |
| | | | KTHTCPPCPAPELLGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALPAP |
| | | | IEKTISKAKGQPREPQVYTLPPSREEMTKNQVSLTCLV |
| | | | KGFYPSDIAVEWESNGQPENNYKTTPPVLDSDGSFFLY |
| | | | SKLTVDKSRWQQGNVFSCSVMHEALHNHYTQKSLSL |
| | | | SPGK |
| 39 | VH5 Full- | PRT | EVQLVESGGGLVQPGGSLKLSCAASGFTFSTFGIHWV |
| | length heavy | | RQAPGKGLEWVAYISGASTTIYYADSVKGRFTISRDNS |
| | chain | | KNTLYLQMNSLRAEDTAVYYCARYGTSFPYGLDYWG |
| | | | QGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSSL |
| | | | GTQTYICNVNHKPSNTKVDKRVEPKSCDKTHTCPPCP |
| | | | APELLGGPSVFLFPPKPKDTLMISRTPEVTCVVVDVSH |
| | | | EDPEVKFNWYVDGVEVHNAKTKPREEQYNSTYRVVS |
| | | | VLTVLHQDWLNGKEYKCKVSNKALPAPIEKTISKAKG |
| | | | QPREPQVYTLPPSREEMTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| 40 | VK3 Full- | PRT | DIVMTQSPDSLAVSLGERATINCKASQSVDYDGDSYM |
| | length light | | NWYQQKPGKAPKLLIYGASNLESGIPDRESGSGSGTDF |
| | chain | | TLTISRLEPEDAAVYYCQQSNEDPFTFGQGTKLEIKRT |
| | | | VAAPSVFIFPPSDEQLKSGTASVVCLLNNFYPREAKVQ |
| | | | WKVDNALQSGNSQESVTEQDSKDSTYSLSSTLTLSKA |
| | | | DYEKHKVYACEVTHQGLSSPVTKSFNRGEC |
| 41 | Human EPH | PRT | MRGSGPRGAGRRRPPSGGGDTPITPASLAGCYSAPRR |
| | receptor A5 | | APLWTCLLLCAALRTLLASPSNEVNLLDSRTVMGDLG |
| | protein | | WIAFPKNGWEEIGEVDENYAPIHTYQVCKVMEQNQN |
| | | | NWLLTSWISN |
| | | | EGASRIFIELKFTLRDCNSLPGGLGTCKETFNMYYFES |
| | | | DDQNGRNIKENQYIKIDTIAADESFTELDLGDRVMKL |
| | | | NTEVRDVGPLSKKGFYLAFQDVGACIALVSVRVYYK |
| | | | KCPSVVRHLAVFPDTITGADSSQLLEVSGSCVNHSVTD |
| | | | EPPKMHCSAEGEWLVPIGKCMCKAGYEEKNGTCQVC |
| | | | RPGFFKASPHIQSCGKCPPHSYTHEEASTSCVCEKDYF |
| | | | RRESDPPTMACTRPPSAPRNAISNVNETSVFLEWIPPA |
| | | | DTGGRKDVSYYIACKKCNSHAGVCEECGGHVRYLPR |
| | | | QSGLKNTSVMMVDLLAHTNYTFEIEAVNGVSDLSPGA |
| | | | RQYVSVNVTTNQAAPSPVTNVKKGKIAKNSISLSWQE |
| | | | PDRPNGIILEYEIKYFEKDQETSYTIIKSKETTITAEGLK |
| | | | PASVYVFQIRARTAAGYGVFSRRFEFETTPVFAASSDQ |
| | | | SQIPVIAVSVTVGVILLAVVIGVLLSGSCCECGCGRASS |
| | | | LCAVAHPSLIWRCGYSKAKQDPEEEKMHFHNGHIKLP |
| | | | GVRTYIDPHTYEDPNQAVHEFAKEIEASCITIERVIGAG |
| | | | EFGEVCSGRLKLPGKRELPVAIKTLKVGYTEKQRRDF |
| | | | LGEASIMGQFDHPNIIHLEGVVTKSKPVMIVTEYMENG |
| | | | SLDTFLKKNDGQFTVIQLVGMLRGISAGMKYLSDMG |
| | | | YVHRDLAARNILINSNLVCKVSDFGLSRVLEDDPEAA |
| | | | YTTRGGKIPIRWTAPEAIAFRKFTSASDVWSYGIVMW |
| | | | EVVSYGERPYWEMTNQDVIKAVEEGYRLPSPMDCPA |
| | | | ALYQLMLDCWQKERNSRPKFDEIVNMLDKLIRNPSSL |
| | | | KTLVNASCRVSNLLAEHSPLGSGAYRSVGEWLEAIKM |
| | | | GRYTEIFMENGYSSMDAVAQVTLEDLRRLGVTLVGH |
| | | | QKKIMNSLQEMKVQLVNGMVPL |
| 42 | VH0-6 | PRT | GFTFSTFGIH |
| | CDR1 | | |

### Exemplary Features

The binding polypeptides, including antibodies and antigen-binding fragments thereof, of the invention are characterized by particular functional features or properties of the antibodies.

In some embodiments, the antibodies or antigen-binding fragments thereof specifically bind to EphA5 protein. In some embodiments, the antibodies or antigen-binding fragments thereof specifically bind to human EphA5. The observation that an antibody or other binding molecule binds to EphA5 protein or specifically binds to EphA5 protein does not necessarily mean that it binds to an EphA5 protein of every species. For example, in some embodiments, features of binding to EphA5 protein, such as the ability to specifically bind thereto and/or to bind with a particular affinity to a particular degree, in some embodiments, refers to the ability with respect to a human EphA5 protein and the antibody may not have this feature with respect to a EphA5 protein of another species, such as mouse.

In some embodiments, the antibodies specifically bind to human EphA5 protein, such as to an epitope or region of human EphA5 protein, such as the human BCMA protein comprising the amino acid sequence of SEQ ID NO: 41 (NCBI No. NP_004430.4) or an allelic variant or splice variant thereof.

In some embodiments, the antibodies or antigen-binding fragment thereof, binds, e.g., specifically binds, and/or recognizes, one or more epitopes in EphA5, e.g., human EphA5. In some embodiments, the epitopes are epitopes present on the extracellular domain of EphA5, e.g., human EphA5. In some embodiments, the epitopes include peptide epitopes. In some embodiments, the epitope includes linear epitopes or conformational epitopes or combination thereof.

In some embodiments, the epitope recognized by a provided antibody includes an epitope containing amino acid residues R306, F309, K321, T328, and H329, with reference to numbering of SEQ ID NO: 41. In some embodiments, the epitope recognized by a provided antibody includes an epitope containing amino acid residues R306, G308, F309, K321, T328, and H329, with reference to numbering of SEQ ID NO: 41. In some embodiments, the epitope recognized by a provided antibody includes an epitope containing amino acid residues R306, G308, F309, K321, T328, H329 and E330, with reference to numbering of SEQ ID NO: 41. In some embodiments, the epitope recognized by a provided antibody includes an epitope containing amino acid residues R306, G308, F309, K321, Y327, T328, H329 and E330, with reference to numbering of SEQ ID NO: 41. In some embodiments, the epitope is a contiguous sequence of amino acids including the above amino acid residues. In some embodiments, a provided antibody or antigen-binding fragment does not bind to an epitope that includes amino acid residues R413, R417 and/or D430.

In provided embodiment, the extent of binding of an anti-EphA5 antibody to an unrelated, non-EphA5 protein, such as a non-human EphA5 protein or other non-EphA5 protein, is less than at or about 10% of the binding of the antibody to human EphA5 protein as measured, e.g., by a radioimmunoassay (RIA).

In some embodiments, the provided antibodies are capable of binding EphA5 protein, such as human EphA5 protein or other EphA5 protein containing an epitope recognized by a provided antibody, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof) specifically binds to a particular ligand (e.g., an antigen, such as a EphA5 protein). It is within the level of a skilled artisan to determine the binding affinity of a binding molecule, e.g., an antibody, for an antigen, e.g., EphA5, such as human EphA5, such as by using any of a number of binding assays that are well known in the art. For example, in some embodiments, a surface plasmon resonance can be used to determine the binding kinetics and constants of a complex between two proteins (e.g., an antibody or fragment thereof, and an antigen, such as a EphA5 protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed nucleic acids or binding of proteins.

In some embodiments, the binding molecule, e.g., antibody or fragment thereof, binds, such as specifically binds, to an antigen, e.g., a EphA5 protein or an epitope therein, with a binding affinity with a K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the antibody or fragment thereof binds, such as specifically binds, to an antigen, e.g., a EphA5 protein or an epitope therein, with a binding affinity with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, the binding molecule, e.g., antibody or fragment thereof, binds, such as specifically binds, to an antigen, e.g., an EphA5 protein or an epitope therein, with a K_{D} of 1.3×10⁻⁹ M or less. In some embodiments, the dissociation constant (K_{D}) for binding to human EpHA5 is less than 1.25×10⁻⁹ M. In some embodiments, the K_{D} is at or about 1.2×10⁻⁹ M, 1.1×10⁻⁹ M, 1.0×10⁻⁹ M, 9.5×10⁻¹⁰ M, 9.0×10⁻¹⁰ M or 8.5×10⁻¹⁰ M, including any values between any of the foregoing. In some embodiments, the dissociation constant (K_{D}) for binding to human EpHA5 is between 8×10⁻¹⁰ M and 1.1×10⁻⁹ M. In some embodiments, the K_{D} is at or about 1.0 nM±0.1 nM. In particular embodiments, binding affinity is determined using surface plasmon resonance.

In some embodiments, properties or features of the provided antibodies (e.g., antigen-binding fragments) are described in relation to properties observed for another antibody, e.g., a reference antibody. In some embodiments, the reference antibody is a non-human anti-EphA5 antibody, such as a murine anti-EphA5 antibody. In some aspects, the reference antibody is the murine antibody designated 11C12, and/or a fragment derived therefrom such as an scFv fragment thereof, and/or an antibody containing the V_{H} and V_{L} regions of such an antibody and/or the heavy and light chain CDRs of such an antibody. For example, in some embodiments, the reference antibody has a V_{H} region containing the amino acid sequence set forth in SEQ ID NO: 1 and a V_{L} region containing the amino acid sequence set forth in SEQ ID NO: 8. In some embodiments, the reference antibody may be a fully murine anti-EphA5 antibody further containing a murine constant domain. In some embodiments, the reference antibody may be a chimeric antibody that contains a heavy chain comprising a murine VH region containing the amino acid sequence set forth in SEQ ID NO: 1 and a heavy chain constant domain set forth in SEQ ID NO: 14, and a light chain comprising a light chain variable domain containing the amino acid sequence set forth in SEQ ID NO: 8 and a light chain constant domain consisting of the amino acid sequence set forth in SEQ ID NO: 15. Typically, it is understood that comparison of binding or other feature or property of a provided antibody to the reference antibody is to the corresponding form or same form of the reference antibody. A “corresponding form” of an antibody means that when comparing a property or activity of two antibodies, the property is compared using the same form (e.g. full-length or Fab or other antigen-binding fragment form) of the antibody. Hence, reference to “corresponding form” and “same form” herein are used interchangeably. For example, if it is stated that an antibody has greater activity compared to the activity of the corresponding form of a first antibody (e.g. reference antibody), that means that a particular form, such as a Fab of that antibody, has greater activity compared to the Fab form of the first antibody.

In some embodiments, among the provided antibodies, such as full length antibodies or antigen-binding fragments thereof, are antibodies in which the dissociation constant (K_{D}) for binding to human EphA5 is more than 1.2 fold better (e.g. lower K_{D}) than the reference antibody (e.g. 11C12, such as an antibody containing the V_{H} and V_{L} set forth in SEQ ID NOs: 1 and 8, respectively). In some embodiments, the K_{D} for binding to human EphA5 is at or about 1.2-fold, 1.3-fold, 1.4-fold, 1.5-fold, 1.6-fold, 1.7-fold, 1.8-fold, 1.9-fold, 2.0-fold or better (e.g. lower K_{D}) than the reference antibody.

Among the provided antibodies (e.g., antigen-binding fragments) are those that compete for binding with and/or bind to the same or overlapping epitopes of EphA5 protein as those bound by the reference antibody described herein but contain a distinct V_{H} and V_{L} region. In some embodiments, among provided antibodies are antibodies that contain distinct set of CDRs, e.g., distinct heavy and light chain CDR1, CDR2, and CDR3.

In some embodiments, the provided binding molecules, such as antibodies or antigen-binding fragments thereof, bind to cell surface EphA5, such as an EphA5 that is a transmembrane protein expressed on the outer membrane of the cell. In some embodiments, the provided binding molecules, such as antibodies or antigen-binding fragments thereof, display a binding preference for EphA5-expressing cells as compared to EphA5-negative cells, such as particular cells known and/or described herein to express EphA5 and known not to express EphA5. The EphA5 expressing cell may be a primary cell or a cell line. An exemplary EphA5 (e.g. human EphA5) expressing cell is the H460 cell line. In some embodiments, the binding preference is observed where a significantly greater degree of binding is measured to the EphA5-expressing, as compared to the non-expressing cells. In some embodiments, the fold change in degree of binding (e.g. EC50) detected, for example, as measured by mean fluorescence intensity in a flow cytometry-based assay and/or dissociation constant or EC₅₀, to the EphA5-expressing cells as compared to the non-EphA5-expressing cells, is at least 1.5, 2, 3, 4, 5, 6, or more, and/or is about as great, about the same, at least as great or about as great, or greater, than the fold change observed for the corresponding form of the reference antibody. In some cases, the total degree of observed binding to EphA5 or to the EphA5-expressing cells is approximately the same, at least as great as, or greater than that observed for the corresponding form of the reference antibody

In some embodiments, the provided binding molecules, such as antibodies or antigen-binding fragments, are capable of being internalized by the cell into which it binds, such as an EphA5-expressing cell. Hence, a provided binding molecule, such as a provided antibody or antigen-binding fragment thereof, is capable of being taken through the cell's lipid bilayer membrane to an internal compartment (i.e. “internalized”) upon binding to the cell. For example, an internalizing anti-EphA5 antibody is one that is capable of being taken into the cell after binding to EphA5 on the cell membrane.

In some embodiments, the binding molecule, such as a provided antibody or antigen-binding fragment thereof, when administered to a subject (e.g. alone or as a conjugate) results in a treatment that does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment, such as binding molecule containing the reference antibody (e.g. 11C12, such as an antibody containing the V_{H} and V_{L} set forth in SEQ ID NOs: 1 and 8, respectively). For example, in the case of an antibody drug conjugate (ADC) including the provided anti-EphA5 antibodies, the degree of immunogenicity is reduced compared to a similar ADC including a different antibody that binds to a similar, e.g., overlapping epitope and/or that competes for binding to EphA5 with the provided antibody, such as a mouse antibody. For instance, in provided embodiments, the 20 degree of immunogenicity of a ADC containing a provided anti-EphA5 antibody is reduced compared to similar ADC but that contains a reference antibody (e.g. 11C12, such as an antibody containing the V_{H} and V_{L} set forth in SEQ ID NOs: 1 and 8, respectively).

In some embodiments, a provided binding molecule, such as an antibody or antigen-binding fragment, exhibits features that favor stability of the antibody or binding fragment thereof. In some embodiments, the stability of a provided binding molecule, e.g. antibody or antigen-binding fragment, is evidenced by its thermal stability. Detection of thermal stability of a protein can be used to determine the denaturation effectively and thus is used as a stability test for protein during the development of therapeutic proteins. A skilled artisan is familiar with assays to assess thermal stability of a protein. In some embodiments, a provided binding molecule, such as an antibody or antigen-binding fragment thereof, has high thermal stability and therefore is less prone to protein aggregation so that activity and/or purity of the protein is improved.

In some embodiments, the binding molecules, such as antibodies or antigen-binding fragments thereof, have an onset temperature of aggregation (Tagg) that is greater than at or about 67° C., greater than at or about 68° C., greater than at or about 69° C., greater than at or about 70° C., greater than at or about 71° C., or greater than at or about 72° C., or is any value between any of the foregoing. In some embodiments, the Tagg is between at or about 67° C. and at or about 71° C. In some embodiments, the Tagg of the antibody or antigen-binding fragment is at or about 67° C., at or about 68° C., at or about 69° C., at or about 70° C., or at or about 71° C.

In some embodiments, the binding molecules, such as antibodies or antigen-binding fragments thereof, have a Tagg that is increased or improved compared to a reference antibody, such as antibody 11C12, such as an antibody containing the V_{H} and V_{L} set forth in SEQ ID NOs: 1 and 8, respectively. In some embodiments, the Tagg is increased greater than at or about 1° C., greater than at or about 2° C., greater than at or about 3° C., greater than at or about 4° C. or greater than at or about 5° C. compared to a reference antibody (e.g. antibody 11C12, such as an antibody containing the V_{H} and V_{L} set forth in SEQ ID NOs: 1 and 8, respectively).

In some embodiments, the binding molecules, such as antibodies or antigen-binding fragments thereof, have a melting temperature (Tm) for unfolding that is greater than 60° C. In some embodiments, the Tm is greater than at or about 61° C., greater than at or about 62° C., greater than at or about 63° C., greater than at or about 64° C., greater than at or about 65° C., greater than at or about 66° C., greater than at or about 67° C., greater than at or about 68° C., greater than at or about 69° C. or greater than at or about 70° C., or any value between any of the foregoing. In some embodiments, the Tm is between at and about 60° C. and at and about 70° C. In some embodiments, the Tm is at or about 64° C., at or about 65° C., at or about 66° C., at or about 67° C., at or about 68° C., or at or about 69° C.

Among provided binding molecules, such as antibodies or antigen-binding fragments, are those that exhibit improved features related to denaturation. Denaturation is the transition of a protein from the native conformation to an unfolded state and is generally accompanied by a major loss of protein function. As is understood by a skilled artisan, as IgG can be described as a multi-domain protein, the melting curve sometimes shows two transitions, or three transitions, with a first denaturation temperature, Tm1, and a second denaturation temperature Tm2, and optionally a third denaturation temperature Tm3. See e.g. Akazawa-Ogawa et al. Biophys. Rev. 2018, 10:255-258.

In some embodiments, the Tm (e.g. Tm1) is increased or improved compared to a reference antibody, such as antibody 11C12, such as an antibody containing the V_{H} and V_{L} set forth in SEQ ID NOs: 1 and 8, respectively. In some embodiments, the Tm (e.g. Tm1) is increased greater than at or about 2° C., greater than at or about 3° C., greater than at or about 4° C., greater than at or about 5° C., greater than at or about 6° C., greater than at or about 7° C., greater than at or about 8° C., greater than at or about 9° C., or greater than at or about 10° C. compared to a reference antibody (e.g. antibody 11C12, such as an antibody containing the V_{H} and V_{L} set forth in SEQ ID NOs: 1 and 8, respectively). In some embodiments, the Tm (e.g. Tm1) is increased greater than at or about 5° C. compared to a reference antibody (e.g. antibody 11C12, such as an antibody containing the V_{H} and V_{L} set forth in SEQ ID NOs: 1 and 8, respectively). In some embodiments, the Tm (e.g. Tm1) is increased greater than at or about 10° C. compared to a reference antibody (e.g. antibody 11C12, such as an antibody containing the V_{H} and V_{L} set forth in SEQ ID NOs: 1 and 8, respectively).

In some embodiments, the binding molecules, such as antibodies or antigen-binding fragments thereof, have a Tm1 for unfolding that is greater than 60° C. In some embodiments, the Tm1 is greater than at or about 61° C., greater than at or about 62° C., greater than at or about 63° C., greater than at or about 64° C., greater than at or about 65° C., greater than at or about 66° C., greater than at or about 67° C., greater than at or about 68° C., greater than at or about 69° C. or greater than at or about 70° C., or any value between any of the foregoing. In some embodiments, the Tm1 is between at and about 60° C. and at and about 70° C. In some embodiments, the Tm1 is at or about 64° C., at or about 65° C., at or about 66° C., at or about 67° C., at or about 68° C., or at or about 69° C.

In some embodiments, among provided binding molecules, such as antibodies or antigen-binding fragments, are those in which the denaturation curve for unfolding is monophasic such that only a single Tm, i.e. TM1, is observed. In some embodiments, without wishing to be bound by theory, it is believed that this property of such antibodies may reduce the likelihood of aggregation and hence improve purity and/or activity of such an antibody preparation. For instance, typically due to the different domains of an antibody folded and unfolded domains may be present at different denaturation temperatures, which can lead to reduced heat resistance and thereby increase tendency to aggregate or have reduced activity. An antibody preparation with a single transition (e.g. monophasic thermal denaturation) would be expected to exhibit improved thermostability and developability.

### Nucleic Acids and Expression Vectors

The present disclosure also provides a nucleic acid, e.g. polynucleotide, encoding any one of the provided binding molecule, including any one of the provided anti-EpHA5 antibody or antigen-binding fragment thereof, or a chain thereof. The provided nucleic acid of the present disclosure may comprise a polynucleotide sequence encoding a heavy and/or light chain of any one of the antibodies or antigen-binding fragments thereof disclosed herein. In some embodiments, the nucleic acid is an isolated nucleic acid. In some embodiments, the nucleic acid is a recombinant or synthetic nucleic acid. In some embodiments, the nucleic acid is a complementary DNA (cDNA).

In some embodiments, the nucleic acid encodes a heavy chain of an antibody or antigen-binding fragment provided herein, such as any described above. In some embodiments, the nucleic acid encodes a light chain of an antibody or antigen-binding fragment provided herein, such as any described above. In some embodiments, the nucleic acid encodes a heavy chain and a light chain of an antibody or antigen-binding fragment provided herein, such as any described above.

In some embodiments, the heavy chain variable region is encoded by a nucleic acid comprising a polynucleotide sequence having has at least 80% sequence identity to SEQ ID NO: 25 but less than 100% sequence identity to SEQ ID NO: 25. In some embodiments, the heavy chain variable region is encoded by a nucleic acid comprising a polynucleotide sequence having has at least 85% sequence identity to SEQ ID NO: 25 but less than 100% sequence identity to SEQ ID NO: 25. For instance, the heavy chain variable region is encoded by a nucleic acid comprising a polynucleotide sequence having has at least 86%, 87%, 87%, 88%, 89%, 90%, 91%, 92%, 93% 94%, 95% sequence identity to SEQ ID NO: 25 but less than 100% sequence identity to SEQ ID NO: 25. In some embodiments, the light chain variable region is encoded by a nucleic acid comprising a polynucleotide sequence having has at least 80% sequence identity to SEQ ID NO: 32 but less than 100% sequence identity to SEQ ID NO: 32. In some embodiments, the light chain variable region is encoded by a nucleic acid comprising a polynucleotide sequence having has at least 85% sequence identity to SEQ ID NO: 32 but less than 100% sequence identity to SEQ ID NO: 32. For instance, the light chain variable region is encoded by a nucleic acid comprising a polynucleotide sequence having at least 86%, 87%, 87%, 88%, 89%, 90%, 91%, 92%, 93% 94%, 95% sequence identity to SEQ ID NO: 32 but less than 100% sequence identity to SEQ ID NO: 32.

In certain embodiments, the heavy chain variable region is encoded by a nucleic acid comprising a polynucleotide sequence having at least 80%, 85%, 90%, 95%, 96%, 96%, 97%, 98%, or 99% identity to SEQ ID NOs: 26, 27, 28, 29, 30, or 31. In certain embodiments, the heavy chain variable region is encoded by a nucleic acid comprising the polynucleotide sequence set forth in SEQ ID NOs: 26, 27, 28, 29, 30, or 31. In certain embodiments, the heavy chain variable region is encoded by a nucleic acid consisting of the polynucleotide sequence set forth in SEQ ID NOs: 26, 27, 28, 29, 30, or 31.

In certain embodiments, the light chain variable region is encoded by a nucleic acid comprising a polynucleotide sequence having at least 80%, 85%, 90%, 95%, 96%, 96%, 97%, 98%, or 99% identity to the amino acid sequence of the light chain variable region set forth in SEQ ID NOs: 33, 34, 35, 36, and 37. In certain embodiments, the light chain variable region is encoded by a nucleic acid comprising the polynucleotide sequence set forth in SEQ ID NOs: 33, 34, 35, 36, and 37. In certain embodiments, the light chain variable region is encoded by a nucleic acid consisting of a polynucleotide sequence set forth in SEQ ID NOs: 33, 34, 35, 36, and 37.

Tolerable variations of the nucleic acid sequences will be known to those of skill in the art. For example, in some embodiments the nucleic acid comprises a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the nucleotide sequences set forth in SEQ ID NOs: 26, 27, 28, 29, 30, 31, 33, 34, 35, 36, and 37.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 26 and a polynucleotide sequence encoding a light chain variable region that has at least 80%, 85%, 90%, 95%, 96%, 96%, 97%, 98%, or 99% sequence identity to the sequence set forth in SEQ ID NO: 33. In some embodiments, the polynucleotide sequence encoding the heavy chain variable region is set forth in SEQ ID NO: 26 and the polynucleotide sequence encoding the light chain variable region is set forth in SEQ ID NO: 33. In some embodiments, the nucleic acid(s) encode the antibody H1K1, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 27 and a polynucleotide sequence encoding a light chain variable region that has at least 80%, 85%, 90%, 95%, 96%, 96%, 97%, 98%, or 99% sequence identity to the sequence set forth in SEQ ID NO: 33. In some embodiments, the polynucleotide sequence encoding the heavy chain variable region is set forth in SEQ ID NO: 27 and the polynucleotide sequence encoding the light chain variable region is set forth in SEQ ID NO: 33. In some embodiments, the nucleic acid(s) encode the antibody H2K1, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 28 and a polynucleotide sequence encoding a light chain variable region that has at least 80%, 85%, 90%, 95%, 96%, 96%, 97%, 98%, or 99% sequence identity to the sequence set forth in SEQ ID NO: 33. In some embodiments, the polynucleotide sequence encoding the heavy chain variable region is set forth in SEQ ID NO: 28 and the polynucleotide sequence encoding the light chain variable region is set forth in SEQ ID NO: 33. In some embodiments, the nucleic acid(s) encode the antibody H3K1, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 29 and a polynucleotide sequence encoding a light chain variable region that has at least 80%, 85%, 90%, 95%, 96%, 96%, 97%, 98%, or 99% sequence identity to the sequence set forth in SEQ ID NO: 33. In some embodiments, the polynucleotide sequence encoding the heavy chain variable region is set forth in SEQ ID NO: 29 and the polynucleotide sequence encoding the light chain variable region is set forth in SEQ ID NO: 33. In some embodiments, the nucleic acid(s) encode the antibody H4K1, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 30 and a polynucleotide sequence encoding a light chain variable region that has at least 80%, 85%, 90%, 95%, 96%, 96%, 97%, 98%, or 99% sequence identity to the sequence set forth in SEQ ID NO: 33. In some embodiments, the polynucleotide sequence encoding the heavy chain variable region is set forth in SEQ ID NO: 30 and the polynucleotide sequence encoding the light chain variable region is set forth in SEQ ID NO: 33. In some embodiments, the nucleic acid(s) encode the antibody H5K1, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 31 and a polynucleotide sequence encoding a light chain variable region that has at least 80%, 85%, 90%, 95%, 96%, 96%, 97%, 98%, or 99% sequence identity to the sequence set forth in SEQ ID NO: 33. In some embodiments, the polynucleotide sequence encoding the heavy chain variable region is set forth in SEQ ID NO: 31 and the polynucleotide sequence encoding the light chain variable region is set forth in SEQ ID NO: 33. In some embodiments, the nucleic acid(s) encode the antibody H6K1, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 26 and a polynucleotide sequence encoding a light chain variable region that has at least 80%, 85%, 90%, 95%, 96%, 96%, 97%, 98%, or 99% sequence identity to the sequence set forth in SEQ ID NO: 34. In some embodiments, the polynucleotide sequence encoding the heavy chain variable region is set forth in SEQ ID NO: 26 and the polynucleotide sequence encoding the light chain variable region is set forth in SEQ ID NO: 34. In some embodiments, the nucleic acid(s) encode the antibody H1K2, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 27 and a polynucleotide sequence encoding a light chain variable region that has at least 80%, 85%, 90%, 95%, 96%, 96%, 97%, 98%, or 99% sequence identity to the sequence set forth in SEQ ID NO: 34. In some embodiments, the polynucleotide sequence encoding the heavy chain variable region is set forth in SEQ ID NO: 27 and the polynucleotide sequence encoding the light chain variable region is set forth in SEQ ID NO: 34. In some embodiments, the nucleic acid(s) encode the antibody H2K2, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 28 and a polynucleotide sequence encoding a light chain variable region that has at least 80%, 85%, 90%, 95%, 96%, 96%, 97%, 98%, or 99% sequence identity to the sequence set forth in SEQ ID NO: 34. In some embodiments, the polynucleotide sequence encoding the heavy chain variable region is set forth in SEQ ID NO: 28 and the polynucleotide sequence encoding the light chain variable region is set forth in SEQ ID NO: 34. In some embodiments, the nucleic acid(s) encode the antibody H3K2, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 29 and a polynucleotide sequence encoding a light chain variable region that has at least 80%, 85%, 90%, 95%, 96%, 96%, 97%, 98%, or 99% sequence identity to the sequence set forth in SEQ ID NO: 34. In some embodiments, the polynucleotide sequence encoding the heavy chain variable region is set forth in SEQ ID NO: 29 and the polynucleotide sequence encoding the light chain variable region is set forth in SEQ ID NO: 34. In some embodiments, the nucleic acid(s) encode the antibody H4K2, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 30 and a polynucleotide sequence encoding a light chain variable region that has at least 80%, 85%, 90%, 95%, 96%, 96%, 97%, 98%, or 99% sequence identity to the sequence set forth in SEQ ID NO: 34. In some embodiments, the polynucleotide sequence encoding the heavy chain variable region is set forth in SEQ ID NO: 30 and the polynucleotide sequence encoding the light chain variable region is set forth in SEQ ID NO: 34. In some embodiments, the nucleic acid(s) encode the antibody H5K2, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 31 and a polynucleotide sequence encoding a light chain variable region that has at least 80%, 85%, 90%, 95%, 96%, 96%, 97%, 98%, or 99% sequence identity to the sequence set forth in SEQ ID NO: 34. In some embodiments, the polynucleotide sequence encoding the heavy chain variable region is set forth in SEQ ID NO: 31 and the polynucleotide sequence encoding the light chain variable region is set forth in SEQ ID NO: 34. In some embodiments, the nucleic acid(s) encode the antibody H6K2, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 26 and a polynucleotide sequence encoding a light chain variable region that has at least 80%, 85%, 90%, 95%, 96%, 96%, 97%, 98%, or 99% sequence identity to the sequence set forth in SEQ ID NO: 35. In some embodiments, the polynucleotide sequence encoding the heavy chain variable region is set forth in SEQ ID NO: 26 and the polynucleotide sequence encoding the light chain variable region is set forth in SEQ ID NO: 35. In some embodiments, the nucleic acid(s) encode the antibody H1K3, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 27 and a polynucleotide sequence encoding a light chain variable region that has at least 80%, 85%, 90%, 95%, 96%, 96%, 97%, 98%, or 99% sequence identity to the sequence set forth in SEQ ID NO: 35. In some embodiments, the polynucleotide sequence encoding the heavy chain variable region is set forth in SEQ ID NO: 27 and the polynucleotide sequence encoding the light chain variable region is set forth in SEQ ID NO: 35. In some embodiments, the nucleic acid(s) encode the antibody H2K3, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 28 and a polynucleotide sequence encoding a light chain variable region that has at least 80%, 85%, 90%, 95%, 96%, 96%, 97%, 98%, or 99% sequence identity to the sequence set forth in SEQ ID NO: 35. In some embodiments, the polynucleotide sequence encoding the heavy chain variable region is set forth in SEQ ID NO: 28 and the polynucleotide sequence encoding the light chain variable region is set forth in SEQ ID NO: 35. In some embodiments, the nucleic acid(s) encode the antibody H3K3, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 29 and a polynucleotide sequence encoding a light chain variable region that has at least 80%, 85%, 90%, 95%, 96%, 96%, 97%, 98%, or 99% sequence identity to the sequence set forth in SEQ ID NO: 35. In some embodiments, the polynucleotide sequence encoding the heavy chain variable region is set forth in SEQ ID NO: 29 and the polynucleotide sequence encoding the light chain variable region is set forth in SEQ ID NO: 35. In some embodiments, the nucleic acid(s) encode the antibody H4K3, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 30 and a polynucleotide sequence encoding a light chain variable region that has at least 80%, 85%, 90%, 95%, 96%, 96%, 97%, 98%, or 99% sequence identity to the sequence set forth in SEQ ID NO: 35. In some embodiments, the polynucleotide sequence encoding the heavy chain variable region is set forth in SEQ ID NO: 30 and the polynucleotide sequence encoding the light chain variable region is set forth in SEQ ID NO: 35. In some embodiments, the nucleic acid(s) encode the antibody H5K3, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 31 and a polynucleotide sequence encoding a light chain variable region that has at least 80%, 85%, 90%, 95%, 96%, 96%, 97%, 98%, or 99% sequence identity to the sequence set forth in SEQ ID NO: 35. In some embodiments, the polynucleotide sequence encoding the heavy chain variable region is set forth in SEQ ID NO: 31 and the polynucleotide sequence encoding the light chain variable region is set forth in SEQ ID NO: 35. In some embodiments, the nucleic acid(s) encode the antibody H6K3, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 26 and a polynucleotide sequence encoding a light chain variable region that has at least 80%, 85%, 90%, 95%, 96%, 96%, 97%, 98%, or 99% sequence identity to the sequence set forth in SEQ ID NO: 36. In some embodiments, the polynucleotide sequence encoding the heavy chain variable region is set forth in SEQ ID NO: 26 and the polynucleotide sequence encoding the light chain variable region is set forth in SEQ ID NO: 36. In some embodiments, the nucleic acid(s) encode the antibody H1K4, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 27 and a polynucleotide sequence encoding a light chain variable region that has at least 80%, 85%, 90%, 95%, 96%, 96%, 97%, 98%, or 99% sequence identity to the sequence set forth in SEQ ID NO: 36. In some embodiments, the polynucleotide sequence encoding the heavy chain variable region is set forth in SEQ ID NO: 27 and the polynucleotide sequence encoding the light chain variable region is set forth in SEQ ID NO: 36. In some embodiments, the nucleic acid(s) encode the antibody H2K4, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 28 and a polynucleotide sequence encoding a light chain variable region that has at least 80%, 85%, 90%, 95%, 96%, 96%, 97%, 98%, or 99% sequence identity to the sequence set forth in SEQ ID NO: 36. In some embodiments, the polynucleotide sequence encoding the heavy chain variable region is set forth in SEQ ID NO: 28 and the polynucleotide sequence encoding the light chain variable region is set forth in SEQ ID NO: 36. In some embodiments, the nucleic acid(s) encode the antibody H3K4, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 29 and a polynucleotide sequence encoding a light chain variable region that has at least 80%, 85%, 90%, 95%, 96%, 96%, 97%, 98%, or 99% sequence identity to the sequence set forth in SEQ ID NO: 36. In some embodiments, the polynucleotide sequence encoding the heavy chain variable region is set forth in SEQ ID NO: 29 and the polynucleotide sequence encoding the light chain variable region is set forth in SEQ ID NO: 36. In some embodiments, the nucleic acid(s) encode the antibody H4K4, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 30 and a polynucleotide sequence encoding a light chain variable region that has at least 80%, 85%, 90%, 95%, 96%, 96%, 97%, 98%, or 99% sequence identity to the sequence set forth in SEQ ID NO: 36. In some embodiments, the polynucleotide sequence encoding the heavy chain variable region is set forth in SEQ ID NO: 30 and the polynucleotide sequence encoding the light chain variable region is set forth in SEQ ID NO: 36. In some embodiments, the nucleic acid(s) encode the antibody H5K4, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 31 and a polynucleotide sequence encoding a light chain variable region that has at least 80%, 85%, 90%, 95%, 96%, 96%, 97%, 98%, or 99% sequence identity to the sequence set forth in SEQ ID NO: 36. In some embodiments, the polynucleotide sequence encoding the heavy chain variable region is set forth in SEQ ID NO: 31 and the polynucleotide sequence encoding the light chain variable region is set forth in SEQ ID NO: 36. In some embodiments, the nucleic acid(s) encode the antibody H6K4, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 26 and a polynucleotide sequence encoding a light chain variable region that has at least 80%, 85%, 90%, 95%, 96%, 96%, 97%, 98%, or 99% sequence identity to the sequence set forth in SEQ ID NO: 37. In some embodiments, the polynucleotide sequence encoding the heavy chain variable region is set forth in SEQ ID NO: 26 and the polynucleotide sequence encoding the light chain variable region is set forth in SEQ ID NO: 37. In some embodiments, the nucleic acid(s) encode the antibody H1K5, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 27 and a polynucleotide sequence encoding a light chain variable region that has at least 80%, 85%, 90%, 95%, 96%, 96%, 97%, 98%, or 99% sequence identity to the sequence set forth in SEQ ID NO: 37. In some embodiments, the polynucleotide sequence encoding the heavy chain variable region is set forth in SEQ ID NO: 27 and the polynucleotide sequence encoding the light chain variable region is set forth in SEQ ID NO: 37. In some embodiments, the nucleic acid(s) encode the antibody H2K5, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 28 and a polynucleotide sequence encoding a light chain variable region that has at least 80%, 85%, 90%, 95%, 96%, 96%, 97%, 98%, or 99% sequence identity to the sequence set forth in SEQ ID NO: 37. In some embodiments, the polynucleotide sequence encoding the heavy chain variable region is set forth in SEQ ID NO: 28 and the polynucleotide sequence encoding the light chain variable region is set forth in SEQ ID NO: 37. In some embodiments, the nucleic acid(s) encode the antibody H3K5, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 29 and a polynucleotide sequence encoding a light chain variable region that has at least 80%, 85%, 90%, 95%, 96%, 96%, 97%, 98%, or 99% sequence identity to the sequence set forth in SEQ ID NO: 37. In some embodiments, the polynucleotide sequence encoding the heavy chain variable region is set forth in SEQ ID NO: 29 and the polynucleotide sequence encoding the light chain variable region is set forth in SEQ ID NO: 37. In some embodiments, the nucleic acid(s) encode the antibody H4K5, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 30 and a polynucleotide sequence encoding a light chain variable region that has at least 80%, 85%, 90%, 95%, 96%, 96%, 97%, 98%, or 99% sequence identity to the sequence set forth in SEQ ID NO: 37. In some embodiments, the polynucleotide sequence encoding the heavy chain variable region is set forth in SEQ ID NO: 30 and the polynucleotide sequence encoding the light chain variable region is set forth in SEQ ID NO: 37. In some embodiments, the nucleic acid(s) encode the antibody H5K5, such as a full-length humanized antibody or an antigen-binding fragment thereof.

In some embodiments, the provided nucleic acid(s) encoding a binding polypeptide, such as an anti-EphA5 antibody or antigen-binding fragment, comprises a polynucleotide sequence encoding a heavy chain variable region that has at least 80%, 85%, 90%, 95%, 96%, 96%, 97%, 98%, or 99% sequence identity to the sequence set forth in SEQ ID NO: 31 and a polynucleotide sequence encoding a light chain variable region that has at least 80%, 85%, 90%, 95%, 96%, 96%, 97%, 98%, or 99% sequence identity to the sequence set forth in SEQ ID NO: 37. In some embodiments, the polynucleotide sequence encoding the heavy chain variable region is set forth in SEQ ID NO: 31 and the polynucleotide sequence encoding the light chain variable region is set forth in SEQ ID NO: 37. In some embodiments, the nucleic acid(s) encode the antibody H6K5, such as a full-length humanized antibody or an antigen-binding fragment thereof.

Also provided is a nucleic acid(s) encoding a binding polypeptide, such as an antibody or antigen-binding fragment, comprising a heavy chain variable region encoded by a nucleic acid sequence comprising the polynucleotide sequence set forth in SEQ ID NO: 29, and a light chain variable region encoded by a nucleic acid sequence comprising the polynucleotide sequence set forth in SEQ ID NO: 35.

Also provided is a nucleic acid(s) encoding a binding polypeptide, such as an antibody or antigen-binding fragment, comprising a heavy chain variable region encoded by a nucleic acid sequence comprising the polynucleotide sequence set forth in SEQ ID NO: 30, and a light chain variable region encoded by a nucleic acid sequence comprising the polynucleotide sequence set forth in SEQ ID NO: 35.

Another aspect of the invention provides a vector comprising any one of the nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell may be of eukaryotic, prokaryotic, mammalian, or bacterial origin. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6 cells; and NSO cells. In some embodiments, the antibody heavy chains and/or light chains (e.g., V_{H} region and/or V_{L} region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., V_{H} region and/or V_{L} region). For example, in some embodiments, CHO cells produce polypeptides that have a higher level of sialylation than the same polypeptide produced in 293 cells.

A method of producing an antibody or antigen-binding fragment thereof that binds to human EPH receptor A5 is also provided herein, wherein the method comprises culturing the host cell. For recombinant production of the anti-EphA5 antibody, a nucleic acid sequence(s) encoding an antibody, e.g., as described above, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-EphA5 antibody is provided, wherein the method comprises culturing a host cell comprising a nucleic acid sequence encoding the antibody, as provided above, under conditions suitable for expression of the antibody, and optionally recovering or purifying the antibody from the host cell (or host cell culture medium).

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid is in operable linkage with a promoter. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; phosphoglycerate kinase-1 (PGK) promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the nucleic acid into a host cell. Accordingly, an expression vector (e.g., a lentiviral vector) of the present invention may comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In some embodiments, the vector further comprises an elongation-factor-1-alpha promoter (EF-1α promoter). Use of an EF-1α promoter may increase the efficiency in expression of downstream transgenes. Physiologic promoters (e.g., an EF-1α promoter) may be less likely to induce integration mediated genotoxicity and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a post-transcriptional regulatory element. Post-transcriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various post-transcriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In some embodiments, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a polypeptide of the present disclosure. Methods for introducing RNA into a host cell are known in the art. See, e.g., Zhao et al. Cancer Res. (2010) 15: 9053. Introducing RNA comprising a nucleotide sequence encoding a polypeptide of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

In order to assess the expression of a polypeptide (e.g. an antibody) or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of an antibody or antigen-binding fragment as described herein, e.g., in a host cell. In some embodiments, a nucleic acid of the present disclosure provides for amplification of the polypeptide-encoding nucleic acid.

### Antibody Drug Conjugates (ADCs)

Also provided in the invention are immunoconjugates, such as antibody drug conjugates (ADCs), comprising any of the antibodies or antigen-binding fragments thereof disclosed herein linked or conjugated to a drug (e.g. a cytotoxic drug), or a toxin, or a radioisotope molecule. In particular embodiments, the ADC is composed of an antibody or antigen-binding fragment disclosed herein and a cytotoxic drug, such as MMAE or SN38. In certain embodiments, the ADCs of the invention can be specifically targeted to human EPH receptor A5 expressing cells in order to effectively eradicate said cells. In provided embodiments, the ADC is able to be internalized by the cell upon binding of the antibody or antigen-binding fragment to EphA5, such as human EphA5.

Exemplary antibodies or antigen-binding fragments in a provided ADC include any as described herein, such as in section “Antibodies and Antigen-Binding Fragments.”

In some embodiments, the immunoconjugate, such as an ADC, has the formula Ab-(L-D), wherein Ab is the antibody or antigen-binding fragment thereof, such as any described herein; L is a linker; and D is a cytotoxic drug.

In some embodiments, the immunoconjugate, such as an ADC, has the formula Ab-(L-D)x, wherein Ab is the antibody or antigen-binding fragment thereof, such as any described herein; L is a linker; and D is a cytotoxic drug and x indicates the number of copies of (L-D) bonded to Ab and is from 1 to 8.

In certain embodiments, the ADC comprises an antibody or antigen-binding fragment thereof comprising a heavy chain variable region comprising the amino acid sequence set forth in SEQ ID NO: 5 and a light chain variable region comprising the amino acid sequence set forth in SEQ ID NO: 11. In some embodiments, the ADC is a full-length antibody in which the heavy chain additionally includes a heavy chain human constant domain (e.g. SEQ ID NO: 14) and the light chain additionally includes a light chain human constant domain (e.g. SEQ ID NO: 15). In some embodiments, the ADC comprises an antibody comprising a heavy chain set forth in SEQ ID NO: 38 and a light chain set forth in SEQ ID NO: 40.

In certain embodiments, the ADC comprises an antibody or antigen-binding fragment thereof comprising a heavy chain variable region comprising the amino acid sequence set forth in SEQ ID NO: 6 and a light chain variable region comprising the amino acid sequence set forth in SEQ ID NO: 11. In some embodiments, the ADC is a full-length antibody in which the heavy chain additionally includes a heavy chain human constant domain (e.g. SEQ ID NO: 14) and the light chain additionally includes a light chain human constant domain (e.g. SEQ ID NO: 15). In some embodiments, the ADC comprises an antibody comprising a heavy chain set forth in SEQ ID NO: 39 and a light chain set forth in SEQ ID NO: 40.

Any of a variety of cytotoxic drugs may be employed as known to a skilled artisan. The antibodies or antigen-binding fragments thereof of the current invention can be linked or conjugated to any drug or cytotoxic agent known to one of skill in the art, including but not limited to maytansinoid (DM1), or SSTR2-binding octreotide, or toxin, including but not limited to paclitaxel, dauxrubicin, duocarmycin A, 5-fluoruracil, methotrexate, tubulin polymerization inhibitors, ravtansine (DM4), and Ricin A. In certain embodiments, the ADCs of the invention may be linked or conjugated to an auristatin including but not limited to monomethyl auristatin E (MMAE) or monomethyl auristatin F (MMAF). In certain embodiments, the ADCs of the invention may be linked or conjugated to an active metabolite of a drug. A nonlimiting example of such drug is SN38, an active metabolite of irinotecan. One of skill in the art would be able to select a drug or cytotoxic agent to conjugate to the antibodies of the invention based on the desired properties of the drug or cytotoxic agent and the properties of the cell to be targeted by the ADC. Exemplary cytotoxic drugs are further described below. In some embodiments, the cytotoxic drug is monomethyl auristatin E (MMAE). In some embodiments, the cytotoxic drug is SN38.

Any of a variety of linkers may be employed as known to a skilled artisan. Exemplary linkers are described below. In some embodiments, the linker is a cleavable linker. In some embodiments, the linker is a cathepsin-cleavable linker. In some embodiments, the linker is a pH cleavable linker.

In some embodiments, the drug antibody ratio (DAR) in a provided conjugate is from at or about 4 to at or about 8. In some embodiments, the DAR is at or about 4. In some embodiments, the DAR is at or about 8.

In certain embodiments, among the provided ADCs are those in which the antibody or antigen-binding fragment is internalized into a target cell upon binding, e.g., into a degradative compartment in the cell. In some embodiments, provided ADCs are thus those that internalize upon binding to a target cell, undergo degradation, and release the drug moiety to kill cancer cells. The drug moiety may be released from the antibody and/or the linker moiety of the ADC by enzymatic action, hydrolysis, oxidation, or any other mechanism. For instance, in some embodiments, the antibody of the ADC binds to EphA5 expressed on the surface of a cell (e.g. cancer cell) and enters the cell upon binding. In some embodiments, the drug moiety of the ADC is released from the antibody moiety of the ADC after the ADC enters and is present in a cell expressing the EphA5 antigen (i.e., after the ADC has been internalized).

### Cytotoxic Drugs

In certain embodiments, the cytotoxic drug is

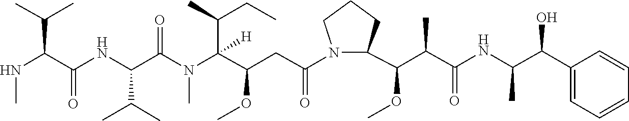 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

(S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, (monomethyl auristatin E (MMAE)).

In certain embodiments, the cytotoxic drug is

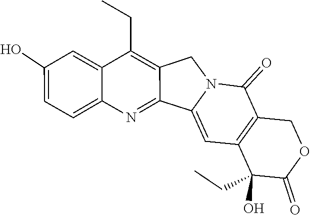 [SMILES: CCc1c2c(nc3ccc(O)cc13)-c1cc3c(c(=O)n1C2)COC(=O)[C@]3(O)CC]

(S)-4,11-diethyl-4,9-dihydroxy-1,12-dihydro-14H-pyrano[3′,4′:6,7]indolizino[1,2-b]quinoline-3,14(4H)-dione, (SN38).

### Linkers

In certain embodiments, the antibody or antigen-binding fragment of the present disclosure (Ab) is conjugated to a cytotoxic drug (D) via a linker (L). In certain embodiments, the linker comprises an antibody conjugating terminus and a carboxy terminus.

In certain embodiments, the antibody conjugating terminus comprises a thiol-conjugating terminus. In certain embodiments, the antibody conjugating terminus comprises a disulfide-conjugating terminus. In certain embodiments, thiol-conjugating terminus comprises a Michael acceptor. In certain embodiments, the thiol-conjugating terminus is conjugated to the antibody via a Michael addition between a Michael acceptor of the linker and a cysteine of the antibody. In certain embodiments, the Michael acceptor is a maleimide. In certain embodiments, the disulfide-conjugating terminus comprises a moiety selected from the group consisting of a bissulfone, 3,4-disubstituted maleimide, bromomaleimide, and/or allyl sulfone. Further examples of disulfide-conjugating moieties are found in Kuan et al. (Chem. Eur. J. 2016, 22(48):17112-17129). In certain embodiments, the bissulfone comprises a 3-(arylsulfonyl)-2-((arylsulfonyl)methyl)propanoyl moiety. In certain embodiments, the disulfide-conjugating terminus is conjugated to the antibody via a reaction with the a 3-(arylsulfonyl)-2-((arylsulfonyl)methyl)propanoyl moiety of the linker and a disulfide of the antibody. See, for example, Bird, Matthew et al. “Bridged Cysteine Conjugations.” Methods in molecular biology (Clifton, N.J.) vol. 2078 (2020): 113-129. doi:10.1007/978-1-4939-9929-3_8.

In certain embodiments, the linker is conjugated to the cytotoxic drug via a carbamate. In certain embodiments, the carbamate is derived from an amine of the cytotoxic drug and a carboxy moiety of the linker. In certain embodiments, the linker is conjugated to the cytotoxic drug via a carbonic ester. In certain embodiments, the carbonic ester is derived from an alcohol moiety of the cytotoxic drug and a carboxy moiety of the linker. In certain embodiments, the linker is conjugated to the cytotoxic drug via an amide. In certain embodiments, the amide is derived from reaction of an amine of the cytotoxic drug and a carboxyl group of the linker. In certain embodiments, the linker is conjugated to more than one cytotoxic drug.

The linkers may comprise ethylene glycol/propylene glycol moieties. For example, in some embodiments both the first linking component and the second linking component (and thus the overall linker) contain ethylene glycol/propylene glycol repeats. Ethylene glycol/propylene glycol-based linkers may provide distinct advantages over linkers not containing ethylene glycol/propylene glycol. For example, ethylene glycol/propylene glycol-based linkers may provide greater flexibility, so that, for example, in an instance where the linker is conjugated to an amino acid residue that is in close proximity to the antigen-binding domain (e.g. the hypervariable region), there would be a reduced chance that the linker/therapeutic agent would bind to or interact with the surface elements of the antigen/receptor, thus making the overall immunoconjugate more therapeutically effective. Additionally, in such embodiments where the linker is generated through click chemistry (e.g. using DBCO and N₃), while not wishing to be bound by theory, the use of ethylene glycol and/or propylene glycol in the linking components may act as a spacer to prevent non-specific interactions (pi-pi interactions) between the tetracyclic ring structure found in the triazole moiety and any exposed aromatic side chains, especially tryptophan side chains. The ethylene glycol/propylene glycol-based linkers of the present disclosure may comprise repeats of anywhere between 1-30 ethylene glycol and/or propylene glycol units total, e.g. 1-10 ethylene glycol and/or propylene glycol units per each of the first linking component and the second linking component in such embodiments that utilize a first linking component and a second linking component.

The covalent linkages in such immunoconjugates may comprise a cleavable linking moiety, for example a cathepsin cleavable linker such as Val-Cit linker or Val-Cit-PAB, which is cleavable by Cathepsin B inside the lysosome. A commercially available Val-Cit linker has been used as described herein. Other cleavable linking moieties may comprise a Phe-Lys linker, which is also cleavable by Cathespin B. Some of the simplest cleavable linking moieties include disulfide (S—S) bridges, which are cleavable in a reductive (i.e. intracellular) environment. However, cleavable linking moieties such as Val-Cit linkers provide more specificity than, for example, disulfide bridges, which may be subject to indiscriminate cleavage, and thus present a superior option, although any such cleavable linking moiety is to be considered within the scope of the present disclosure. An overview of cleavable linking moieties which may be suitable in the context of the present disclosure is provided in Leriche et al. (Bioorg. Med. Chem. 2012, 20(2):571-582), which is hereby incorporated by reference in its entirety.

Alternatively, the linker may be non-cleavable. Non-cleavable linkers may comprise any linking moiety that is resistant to cleavage in an intracellular environment.

In certain aspects of this disclosure, the linker reagent is MC-Val-Cit-PAB, which incorporates the MC (maleimidocaproyl) reactive moiety:

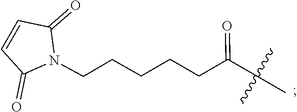 [SMILES: *C(=O)CCCCCN1C(=O)C=CC1=O]

the Val-Cit (valine-citrulline) cleavable moiety:

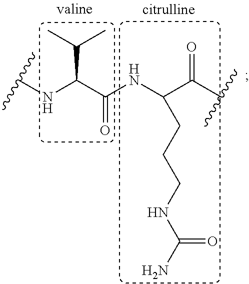 [SMILES: *N[C@H](C(=O)NC(CCCNC(N)=O)C(*)=O)C(C)C]

and the PAB (p-aminobenzyloxycarbonyl) moiety:

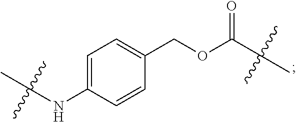 [SMILES: *Nc1ccc(COC(*)=O)cc1]

to form the linker reagent MC-Val-Cit-PAB, also known as MC-VCP, having the structure:

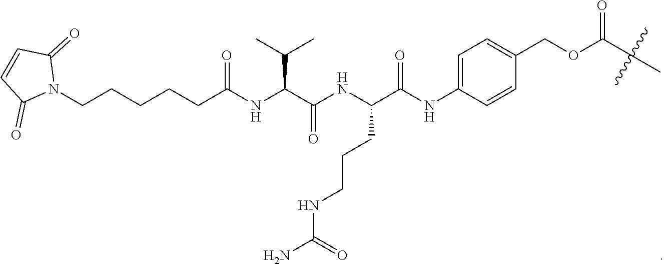 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

wherein the frilled line indicates the attachment point of the cytotoxic drug.

Thus, in certain embodiments, the linker reagent forms a covalent linkage to connect the antibody and the cytotoxic drug to form the antibody drug conjugate and comprises a maleimide linking moiety MC-Val-Cit-PAB having the structure:

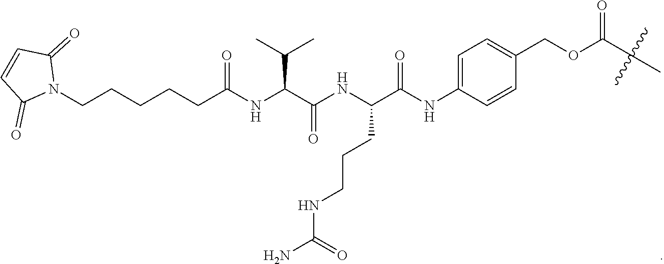 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

wherein the incomplete bond represented by the frilled line represents the attachment point of a cytotoxic drug (D).

In certain embodiments, the maleimide linker reagent comprises CL2A, having the structure:

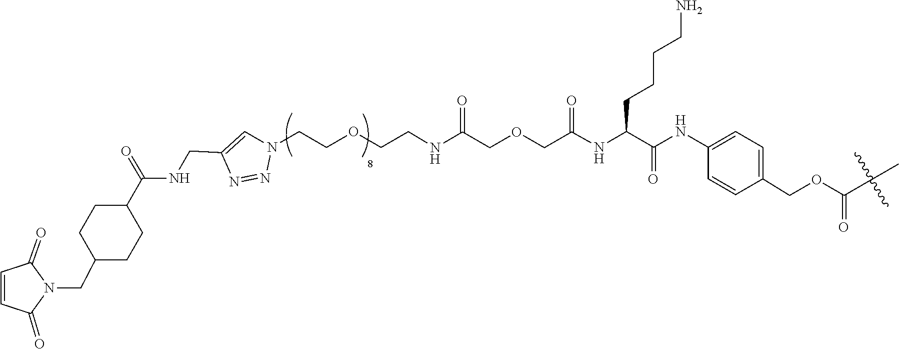

wherein the incomplete bond represented by the frilled line represents the attachment point of a cytotoxic drug (D).

In certain embodiments, the linker reagent that reacts to form the antibody drug conjugate comprises a disulfide crosslinking reagent TBR-Glu-(Val-Cit-PAB)-amino-PEG₂₄-Me having the structure:

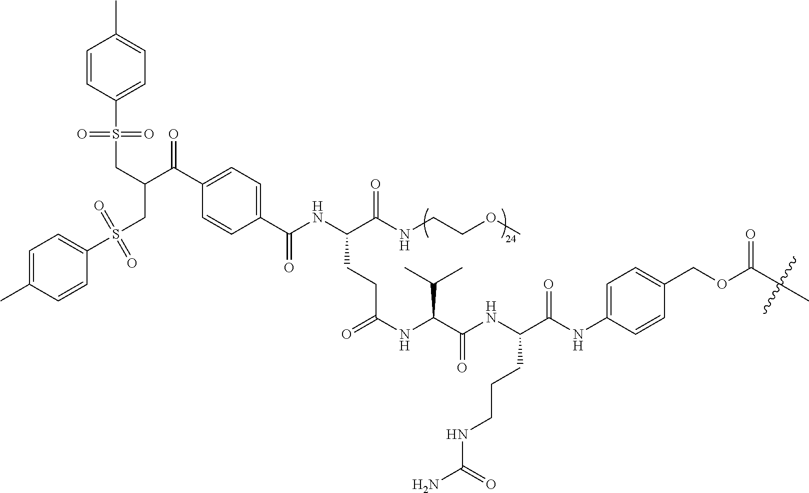

wherein the incomplete bond represented by the frilled line represents the attachment point of a cytotoxic drug (D).

In certain embodiments, the L-D reagent comprises a reactive moiety with a cleavable linker bonded to a MMAE cytotoxin, MC-Val-Cit-PAB-MMAE, having the structure:

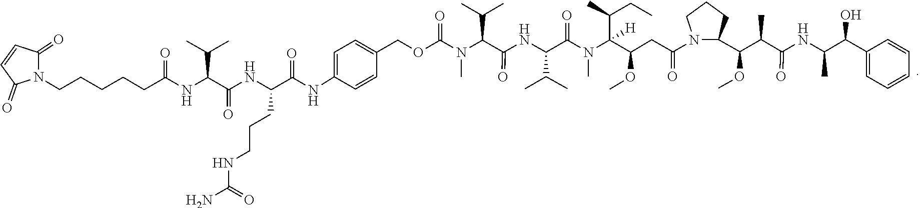 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In certain embodiments, L-D reagent comprises a reactive moiety with a cleavable linker bonded to a cytotoxin, TBR-Glu-(Val-Cit-PAB-MMAE)-amino-PEG₂₄-Me, having the structure:

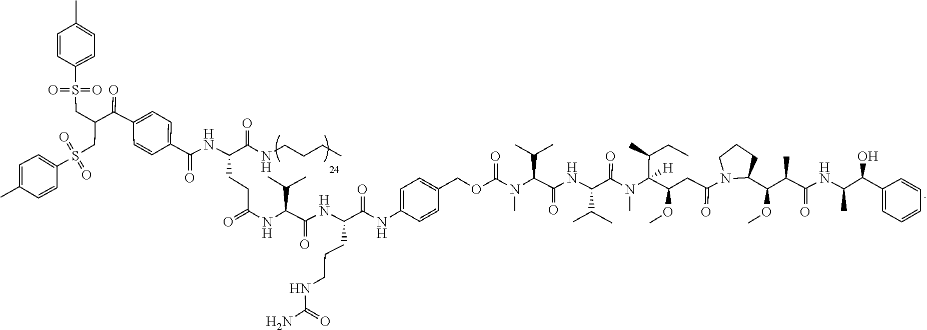

### Antibody-Drug Conjugates

In some embodiments, there is provided an immunoconjugate, such as an ADC, having the formula Ab-(L-D)x, wherein: Ab is an antibody that specifically binds to an epitope of EphA5; and the L-D reagent that reacts with the antibody to form the Ab-(L-D)x comprises one of the structures disclosed above and herein.

In some embodiments, there is provided an immunoconjugate, such as an ADC, having the formula Ab-(L-D)ₓ, wherein Ab is an antibody that specifically binds to an epitope of EphA5, wherein the L-D reagent used to make the ADC is TBR-Glu-(Val-Cit-PAB-MMAE)-amino-PEG₂₄-Me, and the Ab-(L-D)ₓ, wherein x is from 1 to 8, has the structure:

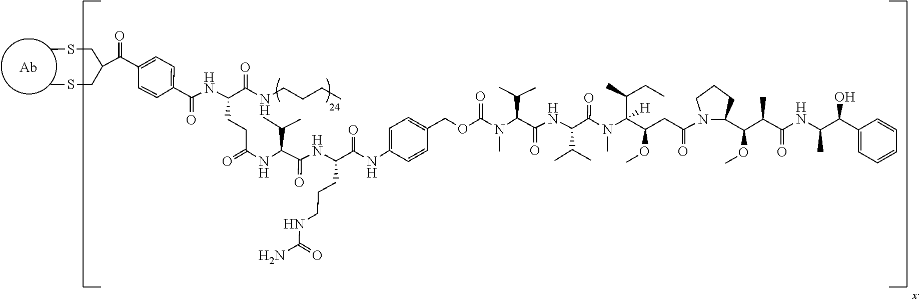

A version of this embodiment wherein the Ab is VH5VK3 and x is 3-5 (predominantly 4), is known as MBRC-101. In certain embodiments x is 4, or x is about 4.

In some embodiments, there is provided an immunoconjugate, such as an ADC, having the formula Ab-(L-D)x, wherein Ab is an antibody that specifically binds to an epitope of EphA5, and the Ab-(L-D)ₓ, wherein the L-D reagent used to make the ADC is TBLR-Glu-(Val-Cit-PAB-MMAE)-amino-PEG₂₄-Me, wherein x is from 1 to 8, having the structure:

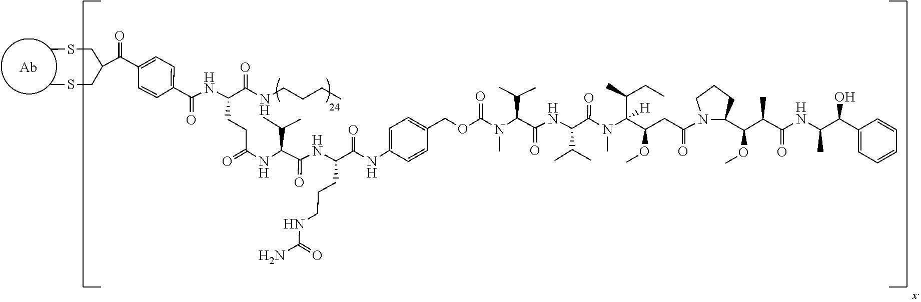

A version of this embodiment (although made using a different linker component compared to the embodiment immediately above) wherein the Ab is VH5VK3 and x is 3-5 (predominantly 4) is likewise known as MBRC-101. In certain embodiments x is 4, or x is about 4.

In some embodiments, there is provided an immunoconjugate, such as an ADC, having the formula Ab-(L-D)x, wherein Ab is an antibody that specifically binds to an epitope of EphA5, the L-D reagent used to make the ADC is TBR-Val-Cit-PAB-MMAE, and the Ab-(L-D)ₓ, wherein x is from 1 to 8, having the structure:

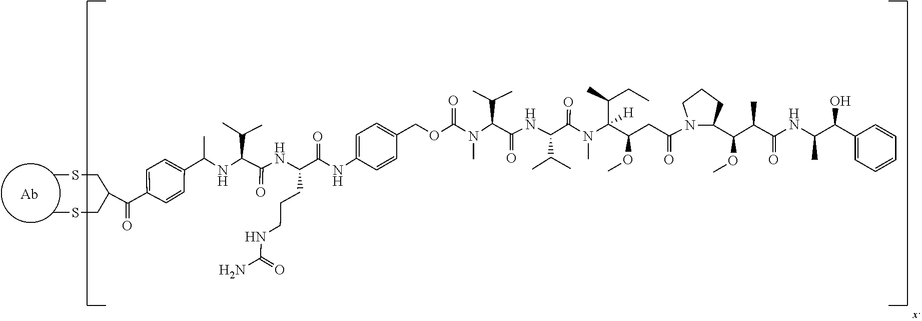

In some embodiments, x is 4 or 8. In some embodiments, x is 1, 2, 3, 4, 5, 6, 7 or 8.

In some embodiments, there is provided an immunoconjugate, such as an ADC of the formula:

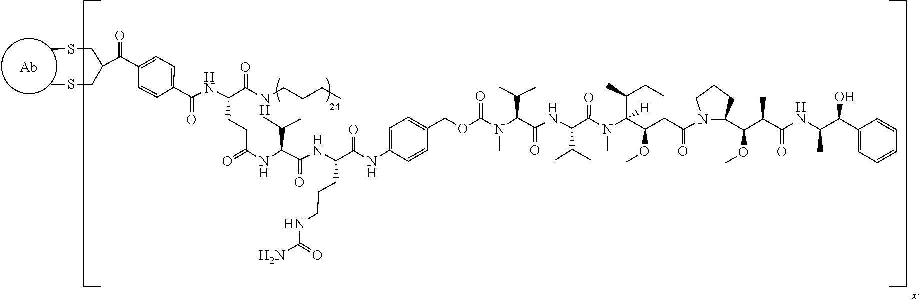

wherein Ab is an antibody that specifically binding to an epitope of human EPH Receptor A5 (EphA5) comprising the heavy chain set forth in SEQ ID NO: 38 and the light chain set forth in SEQ ID NO: 40.

In some embodiments, there is provided an immunoconjugate, such as an ADC of the formula:

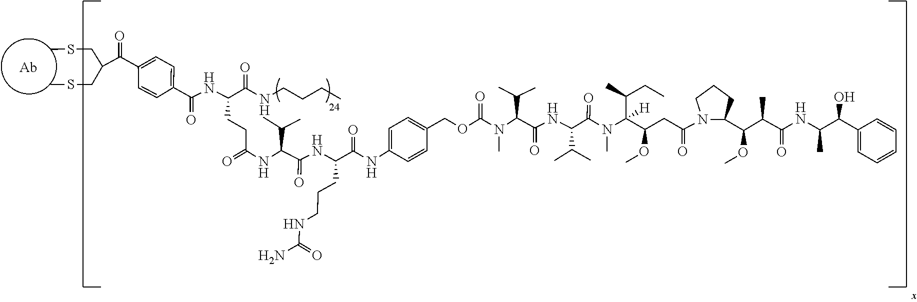

wherein: Ab is an antibody that specifically binding to an epitope of human EPH Receptor A5 (EphA5) comprising the heavy chain set forth in SEQ ID NO: 39 and the light chain set forth in SEQ ID NO: 40.

In some embodiments, there is provided an immunoconjugate, such as an ADC of the formula:

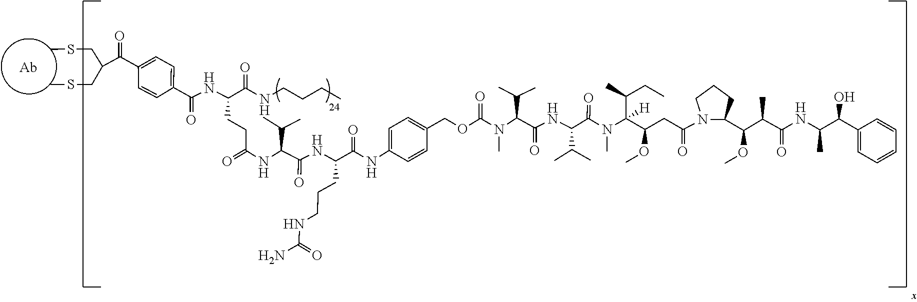

wherein: Ab is an antibody that specifically binding to an epitope of human EPH Receptor A5 (EphA5) comprising the heavy chain set forth in SEQ ID NO: 6 and the light chain set forth in SEQ ID NO: 11.

In some embodiments, there is provided an immunoconjugate, such as an ADC of the formula:

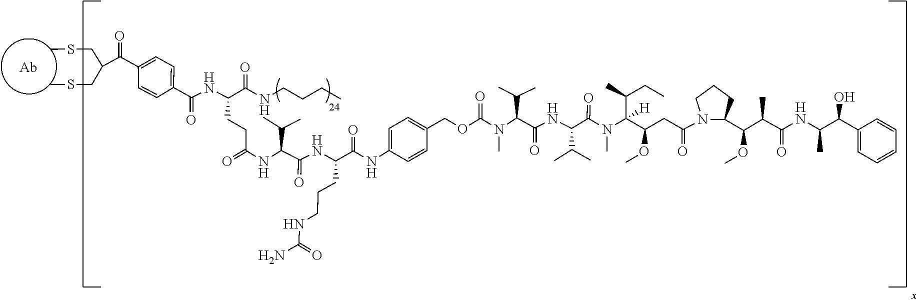

wherein: Ab is an antibody that specifically binding to an epitope of human EPH Receptor A5 (EphA5) comprising:
(a) a first VH CDR set forth in SEQ ID NO: 16;
(b) a second VH CDR set forth in SEQ ID NO: 18;
(c) a third VH CDR set forth in SEQ ID NO: 20;
(d) a first VL CDR set forth in SEQ ID NO: 21;
(e) a second VL CDR set forth in SEQ ID NO: 23; and
(f) a third VL CDR set forth in SEQ ID NO: 24.

In some embodiments, there is provided an immunoconjugate, such as an ADC of the formula:

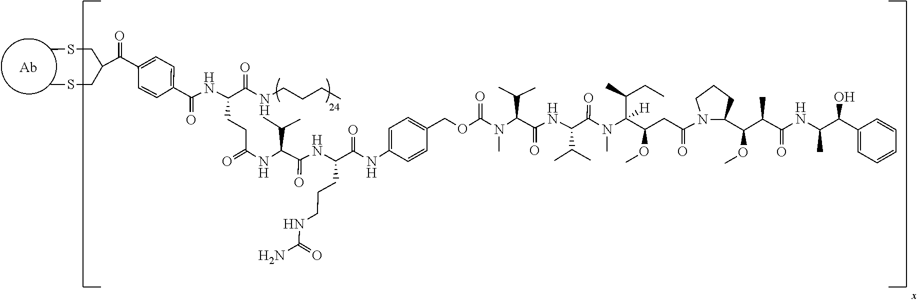

wherein: Ab is an antibody that specifically binding to an epitope of human EPH Receptor A5 (EphA5) comprising additional sequences, and/or combinations of additional sequences, recited in Table 1, above.

Some embodiments include a method for treating, ameliorating, and/or preventing cancer in a subject, wherein the cancer is associated with expression of EPH Receptor A5, the method comprising administering to the subject an effective amount of a composition comprising an ADC described above and/or herein.

Some embodiments include a method for treating, ameliorating, and/or preventing squamous cell carcinoma of the head and neck in a subject, wherein the cancer is associated with expression of EPH Receptor A5, the method comprising administering to the subject an effective amount of a composition comprising an ADC described above and/or herein.

Some embodiments a pharmaceutical composition comprising an ADC described above and/or herein, for use in treating, ameliorating, and/or preventing cancer in a subject in need thereof, wherein the cancer is associated with expression of EPH Receptor A5.

Some embodiments a pharmaceutical composition comprising an ADC described above and/or herein, for use in treating, ameliorating, and/or preventing squamous cell carcinoma of the head and neck in a subject in need thereof, wherein the cancer is associated with expression of EPH Receptor A5.

Other embodiments are also contemplated by this disclosure, as would be understood by one of skill in the art.

### Pharmaceutical Compositions and Formulations

Also provided are pharmaceutical composition comprising any one of the antibodies, antigen-binding fragments, and immunoconjugates (e.g. antibody drug conjugates) disclosed herein. Among the compositions are pharmaceutical compositions and formulations for administration, such as for treatment, amelioration, and/or prevention of a disease or disorder. Also provided are therapeutic methods for administering the pharmaceutical compositions to subjects, e.g., patients.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects the choice of carrier is determined in part by the particular composition and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

In some embodiments, the formulations can include aqueous solutions. In some embodiments, the formulations can include lyophilized formulations.

The formulation or composition may also contain more than one active ingredient useful for the particular indication, disease, or condition being treated, ameliorated, and/or prevented with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as an anti-cancer drug. In some embodiments, the anti-cancer drug is a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the composition in an amount effective to treat, ameliorate, and/or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. The provided compositions may be administered using standard administration techniques, formulations, and/or devices. Provided are formulations and devices, such as syringes and vials, for storage and administration of the compositions.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

Methods of Treatment, Amelioration, and/or Prevention

Also provided are methods, such as methods of treatment and uses for treating, ameliorating, and/or preventing a disease or condition, such as a cancer, with any one of the provided antibodies (including a full-length antibody or antigen-binding fragments thereof) and immunoconjugates (e.g. antibody drug conjugates) described herein. In some embodiments, any one of the provided antibodies, and immunoconjugates (e.g. antibody drug conjugates) may be included in a composition for treating, ameliorating, and/or preventing the disease or condition in a subject in need thereof. The composition may include a pharmaceutical composition and further include a pharmaceutically acceptable carrier. A therapeutically effective amount of the pharmaceutical composition may be administered to the subject.

In some embodiments, the methods include methods of administering and uses, such as therapeutic and prophylactic uses, of any one of the antibodies and immunoconjugates (e.g. antibody drug conjugates) described herein or compositions containing the same. Such methods and uses include therapeutic methods and uses, for example, involving administration any one of the provided antibodies and immunoconjugates (e.g. antibody drug conjugates), or compositions containing the same, to a subject having a disease, condition, or disorder associated with EphA5. Also provided herein are uses of any one of the provided antibodies and immunoconjugates (e.g. antibody drug conjugates), or compositions containing the same, in such methods and treatments, and in the preparation of a medicament in order to carry out such therapeutic methods. In some embodiments, the disease, condition, or disorder is associated with EphA5 expression, and/or in which cells or tissues express, e.g., specifically express EphA5.

In some embodiments, a composition containing a provided antibody or immunoconjugates (e.g. antibody drug conjugates) is administered in an effective amount to effect treatment, amelioration, and/or prevention of the disease or disorder. In some embodiments, the methods are carried out by administering any one of the provided antibodies and immunoconjugates (e.g. antibody drug conjugates), or compositions containing the same, to the subject having, having had, or suspected of having the disease or condition. In some embodiments, the methods thereby treat, ameliorate, and/or prevent the disease or condition or disorder in the subject. Also provided herein are of use of any of the compositions, such as pharmaceutical compositions provided herein, for the treatment, amelioration, and/or prevention of a disease or disorder associated with EphA5, for example, a cancer.

Among the diseases to be treated, ameliorated, and/or prevented is any disease or disorder associated with EphA5, such as any disease or disorder in which BCMA is specifically expressed and/or in which EphA5 has been targeted for treatment (also referred to herein interchangeably as a “EphA5-associated disease or disorder”). In some embodiments, the disease or disorder is a cancer. In certain embodiments, the cancer is associated with human EPH receptor A5 (human EphA5)-expressing cells. In certain embodiments, the EPH receptor A5-expressing cell is a cancer cell.

In some embodiments, the provided antibodies and immunoconjugates (e.g. antibody drug conjugates), and compositions containing the same, can be used to treat, ameliorate, and/or prevent a cancer in which tumor cells express EphA5 on their surface.

In some embodiments, the provided antibodies and immunoconjugates (e.g. antibody drug conjugates), and compositions containing the same, may be used to treat, ameliorate, and/or prevent a carcinoma, specifically a squamous cell carcinoma (e.g., head and neck squamous cell carcinoma).

In some embodiments, the methods may identify a subject who has, is suspected to have, or is at risk for developing a EphA5-associated disease or disorder. Hence, provided are methods for identifying subjects with diseases or disorders associated with elevated EphA5 expression and selecting them for treatment with any one of the provided antibodies and immunoconjugates (e.g. antibody drug conjugates) or compositions containing the same. For example, a subject may be screened for the presence of a disease or disorder associated with elevated EphA5 expression, such as an EphA5-expressing cancer. In some aspects, a sample may be obtained from a patient suspected of having a disease or disorder associated with elevated EphA5 expression and assayed for the expression level of EphA5. In some aspects, a subject who tests positive for a EphA5-associated disease or disorder may be selected for treatment by the present methods, and may be administered a therapeutically effective amount of any one of the provided antibodies and immunoconjugates (e.g. antibody drug conjugates) or compositions containing the same as described herein. In some embodiments, the methods can be used to monitor the size or density of an EphA5-expressing tissue, e.g. tumor, over time, e.g., before, during, or after treatment by the methods.

Compositions of the invention can be administered in dosages and routes and at times to be determined in appropriate pre-clinical and clinical experimentation and trials. Compositions may be administered multiple times at dosages within these ranges. Administration of the compositions may be combined with other methods useful to treat, ameliorate, and/or prevent the desired disease or condition as determined by those of skill in the art.

In some embodiments, a composition herein may contain an effective amount of a provided antibody or immunoconjugate (e.g. antibody drug conjugate) that may be from 0.001 mg to 1000 mg, such as from 0.001 mg to 100 mg, 0.001 mg to 10 mg, 0.001 mg to 1 mg, 0.001 mg to 0.1 mg or 0.001 mg to 0.01 mg. Depending on the type and severity of the disease, dosages of antibody or immunoconjugate (e.g. antibody drug conjugate) may include about 1 μg/kg to about 15 mg/kg (e.g. 0.1 mg/kg-10 mg/kg), about 1 μg/kg to about 100 mg/kg, about 0.05 mg/kg to about 10 mg/kg, about 0.5 mg/kg, about 2.0 mg/kg, about 4.0 mg/kg or about 10 mg/kg. Multiple doses may be administered intermittently, e.g. every week or every three weeks. An initial higher loading dose, followed by one or more lower doses may be administered.

In some embodiments, the antibody may be administered systemically. In additional aspects, the antibody may be administered intravenously, intradermally, intratumorally, intramuscularly, intraperitoneally, subcutaneously, or locally. The method may further comprise administering at least a second anticancer therapy to the subject. Examples of the second anticancer therapy include, but are not limited to, surgical therapy, chemotherapy, radiation therapy, cryotherapy, hormonal therapy, immunotherapy, or cytokine therapy.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, the degree of immunogenicity in some embodiments is reduced compared to a similar antibody drug conjugate including a different antibody that binds to a similar, e.g., overlapping epitope and/or that competes for binding to EphA5 with the provided antibody, such as a reference antibody as described.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

Thus, in some embodiments of the invention there is provided a method for treating, ameliorating, and/or preventing squamous cell carcinoma of the head and neck, colorectal cancer, and/or pancreatic cancer in a subject, the method comprising administering to the subject an effective amount of a composition comprising the components Ab(antibody)-L(linker)-D(drug) as follows:

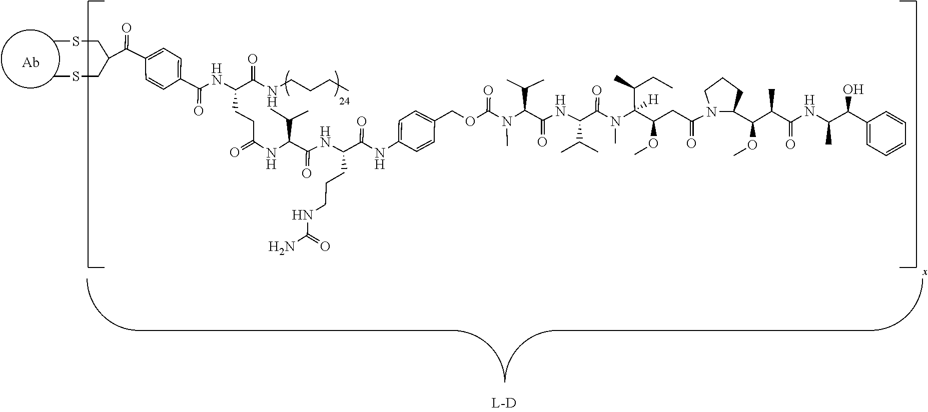

wherein x is an integer from 1 to 8; and wherein Ab is a human or humanized monoclonal antibody comprising: a variable heavy chain comprising at least 90% sequence identity to the amino acid chain set forth in SEQ ID NO: 6 and a variable light chain comprising at least 90% sequence identity to the amino acid chain set forth in SEQ ID NO: 11. In some of these embodiments the variable heavy chain comprises the amino acid chain set forth in SEQ ID NO: 6 and the variable light chain comprises the amino acid chain set forth in SEQ ID NO: 11. In some of these embodiments the composition is administered by injection or infusion at a dose of 0.5 or more mg/kg, 1.0 or more mg/kg, 2.0 or more mg/kg, 4.0 or more mg/kg, or 10.0 or more mg/kg, and it may be administered by injection or infusion twice a week, three time a week, weekly, bi-weekly, every three weeks, or monthly.

In other embodiments, there is provided a method for treating, ameliorating, and/or preventing squamous cell carcinoma of the head and neck, colorectal cancer, and/or pancreatic cancer in a subject, the method comprising administering to the subject an effective amount of a composition comprising the components Ab(antibody)-L(linker)-D(drug) as follows:

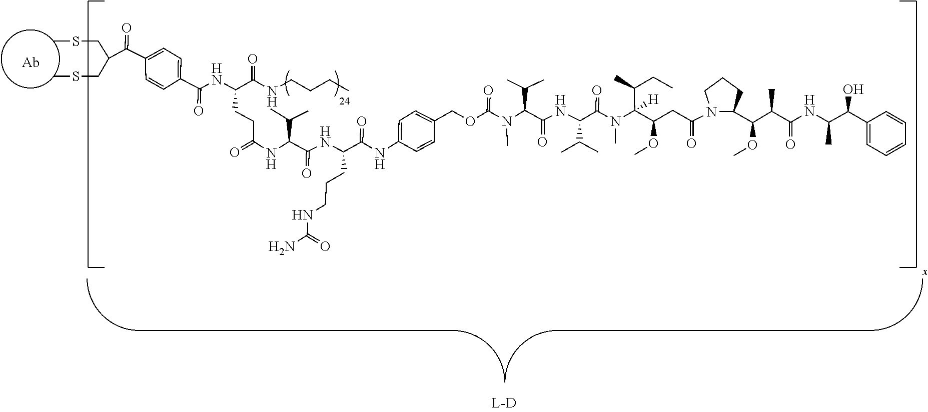

wherein x is an integer from 1 to 8; and wherein Ab is a human or humanized monoclonal antibody comprising: (a) a first VH CDR comprising at least 90% sequence identity to the amino acid chain set forth in SEQ ID NO: 16; (b) a second VH CDR comprising at least 90% sequence identity to the amino acid chain set forth in SEQ ID NO: 18; (c) a third VH CDR comprising at least 90% sequence identity to the amino acid chain set forth in SEQ ID NO: 20; (d) a first VL CDR comprising at least 90% sequence identity to the amino acid chain set forth in SEQ ID NO: 21; (e) a second VL CDR comprising at least 90% sequence identity to the amino acid chain set forth in SEQ ID NO: 23; and (f) a third VL CDR comprising at least 90% sequence identity to the amino acid chain set forth in SEQ ID NO: 24. In certain such embodiments the first VH CDR comprises the amino acid chain set forth in SEQ ID NO: 16; the second VH CDR comprises the amino acid chain set forth in SEQ ID NO: 18; the third VH CDR comprises the amino acid chain set forth in SEQ ID NO: 20; the first VL CDR comprises the amino acid chain set forth in SEQ ID NO: 21; the second VL CDR comprises the amino acid chain set forth in SEQ ID NO: 23; and the third VL CDR comprises the amino acid chain set forth in SEQ ID NO: 24. In such embodiments the composition may be administered by injection or infusion at a dose of 0.5 or more mg/kg, 1.0 or more mg/kg, 2.0 or more mg/kg, 4.0 or more mg/kg, or 10.0 or more mg/kg, and it may be administered by injection or infusion twice a week, three time a week, weekly, bi-weekly, every three weeks, or monthly.

Additional embodiments are provided including a method for treating, ameliorating, and/or preventing squamous cell carcinoma of the head and neck, colorectal cancer, and/or pancreatic cancer in a subject, the method comprising administering to the subject an effective amount of a composition comprising the components Ab(antibody)-L(linker)-D(drug) as follows:

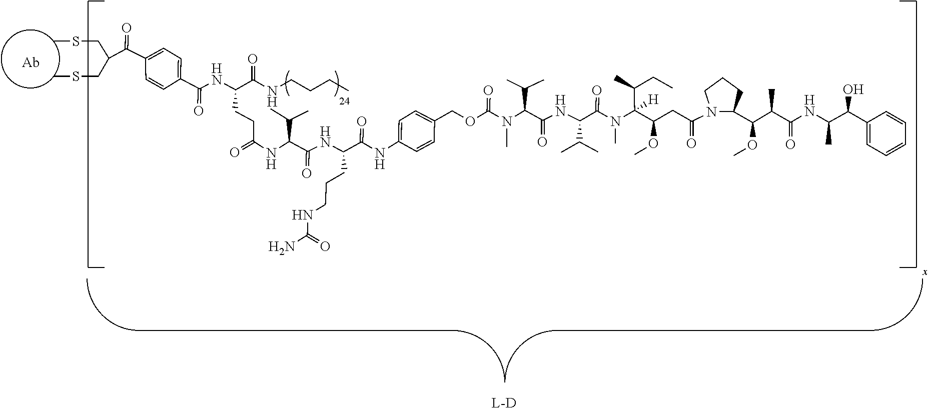

wherein x is an integer from 1 to 8; and wherein Ab is a human or humanized monoclonal antibody comprising: a variable heavy chain comprising at least 90% sequence identity to the amino acid chain set forth in SEQ ID NO: 39; and a variable light chain comprising at least 90% sequence identity to the amino acid chain set forth in SEQ ID NO: 40. In some of these embodiments the variable heavy chain comprises the amino acid chain set forth in SEQ ID NO: 39; and wherein said variable light chain comprises the amino acid chain set forth in SEQ ID NO: 40. In some of these embodiments the composition is administered by injection or infusion at a dose of 0.5 or more mg/kg, 1.0 or more mg/kg, 2.0 or more mg/kg, 4.0 or more mg/kg, or 10.0 or more mg/kg, and in some embodiments the composition is administered by injection or infusion twice a week, three time a week, weekly, bi-weekly, every three weeks, or monthly.

In other embodiments there is provided a pharmaceutical composition for use in treating, ameliorating, and/or preventing squamous cell carcinoma of the head and neck, colorectal cancer, and/or pancreatic cancer in a subject in need thereof, wherein the cancer is associated with expression of EPH Receptor A5, and the composition comprises the components Ab(antibody)-L(linker)-D(drug) as follows:

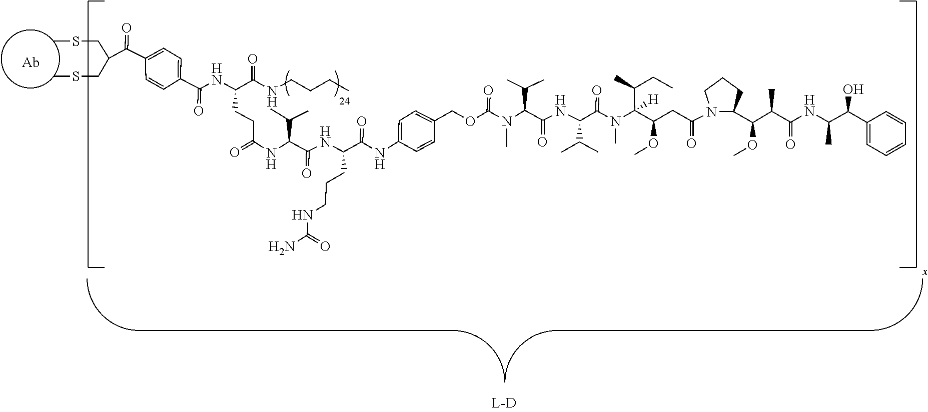

wherein the L-D portion of the composition has an x is from 1 to 8; and wherein the Ab component is a human or humanized monoclonal antibody comprising: a variable heavy chain comprising at least 90% sequence identity to the amino acid chain set forth in SEQ ID NO: 6; and a variable light chain comprising at least 90% sequence identity to the amino acid chain set forth in SEQ ID NO: 11. In some of these embodiments the variable heavy chain comprises the amino acid chain set forth in SEQ ID NO: 6; and the variable light chain comprises the amino acid chain set forth in SEQ ID NO: 11. In some of these embodiments, the composition is formulated to dose by injection or infusion at 0.5 or more mg/kg, 1.0 or more mg/kg, 2.0 or more mg/kg, 4.0 or more mg/kg, or 10.0 or more mg/kg, and in some of these embodiments the composition is formulated for administration twice a week, three time a week, weekly, bi-weekly, every three weeks, or monthly.

In certain embodiments of the invention there is provided a pharmaceutical composition for use in treating, ameliorating, and/or preventing s squamous cell carcinoma of the head and neck, colorectal cancer, and/or pancreatic cancer in a subject in need thereof, wherein the cancer is associated with expression of EPH Receptor A5, and the composition comprises the components Ab(antibody)-L(linker)-D(drug) as follows:

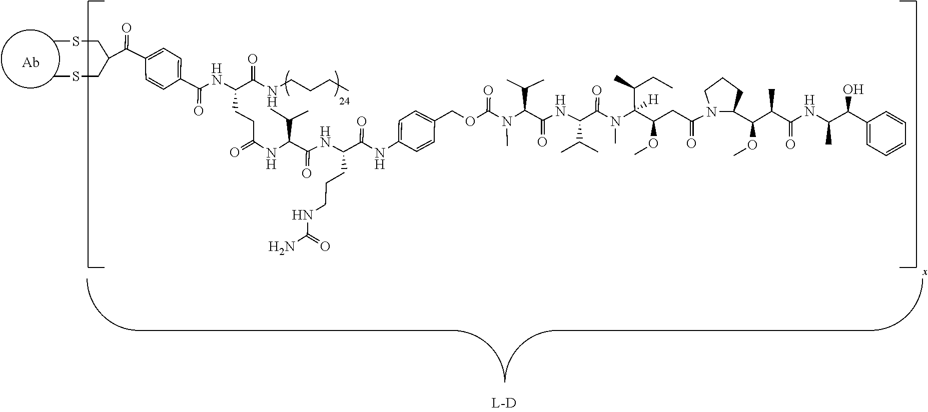

wherein the L-D portion of the composition has an x is from 1 to 8; and wherein the Ab component is a human or humanized monoclonal antibody comprising: a variable heavy chain comprising at least 90% sequence identity to the amino acid chain set forth in SEQ ID NO: 39; and a variable light chain comprising at least 90% sequence identity to the amino acid chain set forth in SEQ ID NO: 40. In some of these embodiments the variable heavy chain comprises the amino acid chain set forth in SEQ ID NO: 39; and the variable light chain comprises the amino acid chain set forth in SEQ ID NO: 40. In some of these embodiments, the composition is formulated to dose by injection or infusion at 0.5 or more mg/kg, 1.0 or more mg/kg, 2.0 or more mg/kg, 4.0 or more mg/kg, or 10.0 or more mg/kg, and in some of these embodiments the composition is formulated for administration twice a week, three time a week, weekly, bi-weekly, every three weeks, or monthly.

In certain embodiments of the invention there is provided a pharmaceutical composition for use in treating, ameliorating, and/or preventing squamous cell carcinoma of the head and neck, colorectal cancer, and/or pancreatic cancer in a subject in need thereof, wherein the cancer is associated with expression of EPH Receptor A5, and the composition comprises the components Ab(antibody)-L(linker)-D(drug) as follows:

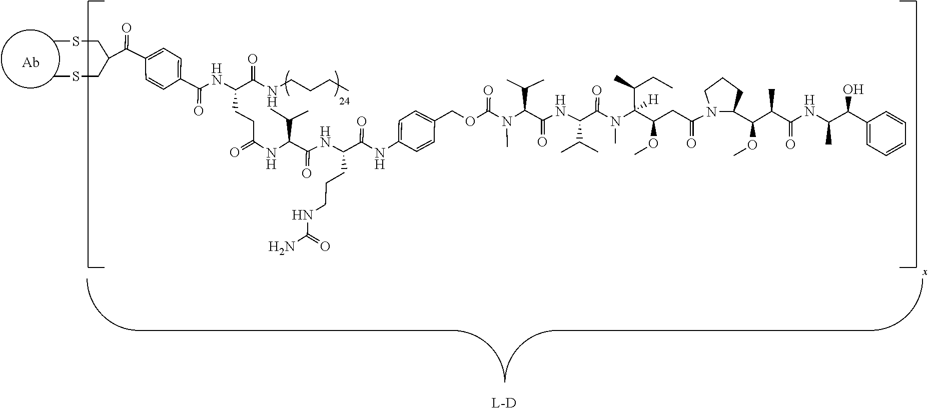

wherein the L-D portion of the composition has an x is from 1 to 8; and wherein the Ab component is a human or humanized monoclonal antibody comprising: (a) a first VH CDR comprising at least 90% sequence identity to the amino acid chain set forth in SEQ ID NO: 16; (b) a second VH CDR comprising at least 90% sequence identity to the amino acid chain set forth in SEQ ID NO: 18; (c) a third VH CDR comprising at least 90% sequence identity to the amino acid chain set forth in SEQ ID NO: 20; (d) a first VL CDR comprising at least 90% sequence identity to the amino acid chain set forth in SEQ ID NO: 21; (e) a second VL CDR comprising at least 90% sequence identity to the amino acid chain set forth in SEQ ID NO: 23; and (f) a third VL CDR comprising at least 90% sequence identity to the amino acid chain set forth in SEQ ID NO: 24. In some of these embodiments, the first VH CDR comprises the amino acid chain set forth in SEQ ID NO: 16; the second VH CDR comprises the amino acid chain set forth in SEQ ID NO: 18; the third VH CDR comprises the amino acid chain set forth in SEQ ID NO: 20; the first VL CDR comprises the amino acid chain set forth in SEQ ID NO: 21; the second VL CDR comprises the amino acid chain set forth in SEQ ID NO: 23; and the third VL CDR comprises the amino acid chain set forth in SEQ ID NO: 24. In some of these embodiments, the composition is formulated to dose by injection or infusion at 0.5 or more mg/kg, 1.0 or more mg/kg, 2.0 or more mg/kg, 4.0 or more mg/kg, or 10.0 or more mg/kg, and in some of these embodiments the composition is formulated for administration twice a week, three time a week, weekly, bi-weekly, every three weeks, or monthly.

While the present invention has been described with reference to the specific embodiments thereof, it should be understood by those skilled in the art that various changes may be made, and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 1: In Vivo Anti-Tumor Activity of ADCs Directed to EphA5 in a Patient-Derived Xenograft Model of Head and Neck Squamous Cell Carcinoma

The objective of this study was to evaluate the therapeutic anti-tumor activity of two ADCs directed to a novel target, EphA5, using a PDX model of head and neck squamous cell carcinoma (SCC). Specifically, the objective of this study was to test the in vivo anti-tumor activity of two ADCs, MBRC-101 and VH5VK3-(SN38)₂, (see SEQ IDs 6 and 11) in a PDX mouse model of head and neck squamous cell carcinoma that has been shown to express EphA5 at high levels on the cell surface of the tumor cells.

The present studies tested the in vivo anti-tumor activity of an antibody-drug conjugate (ADC) targeting the novel cell-surface receptor target EphA5 using a PDX model for head and neck squamous cell carcinoma. Patient-derived tumor xenografts (PDXs), in which tumor fragments are surgically dissected from cancer patients and directly transplanted into immunodeficient mice, have been established as predictive models in translational research. PDX susceptibility to anti-cancer drugs has been closely correlated with clinical data in patients from whom PDX models have been derived, and PDX models often maintain the cellular and histopathological structures of the original tumors (Tentler et al., 2012; Lawson et al., 2015; Xiao et al., 2018). Genetic analysis of tumor cells from PDX models following transplantation has shown preservation of the genomic and gene expression profiles between PDXs and parental patient tumors (Reyal et al., 2012; Zhao et al., 2012; Garcia et al., 2018) and sensitivity to standard chemotherapeutics in PDXs closely correlates with clinical data in patients from which the PDXs are derived (Topp et al., 2014; Nunes et al., 2015; Trusolino et al., 2015; Evans et al., 2017). Over 500 different PDX models have been characterized for 25 different tumor types in a multi-center effort, which has provided comprehensive information on the correlation between these models and various tumor types (Sun et al., 2021). PDX models are thus highly effective in vivo models for predicting the efficacy of anti-cancer therapeutics, especially for solid tumors.

The ADC tested consisted of a monoclonal antibody that selectively binds to human EphA5, a proprietary site-specific conjugation technology, a well-validated linker molecule, and an auristatin payload toxin as described in more detail herein. The development and production of this molecule is as set forth herein and in PCT/US2022/079232, which is incorporated by reference herein for all purposes.

This ADC targeted the tumor cells in the grafted tumor tissue, leading to specific cell death of the cancer cells and regression of the tumors in the treated animals. The study was a small dose-range study that examined the efficacy of different doses of the ADC in the PDX model of head and neck squamous cell carcinoma (SCC).

### 1. Materials

### Test Article 1 (TA1): MBRC-101

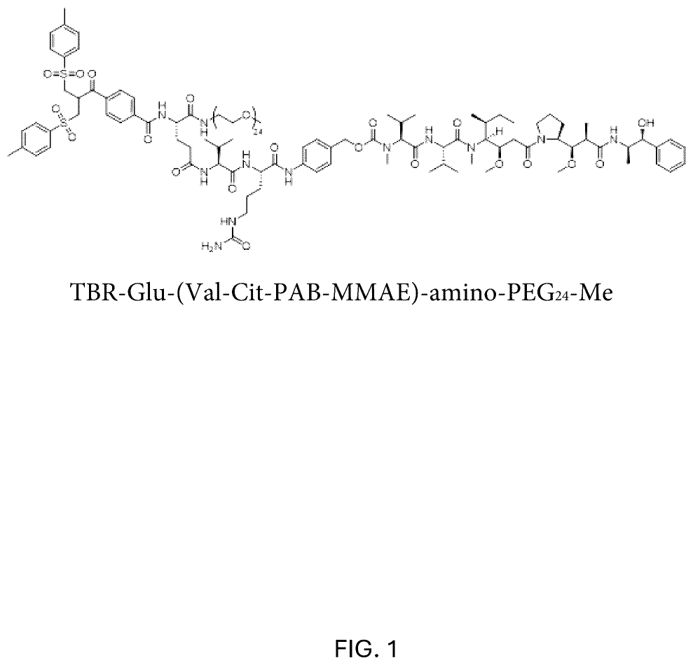

TA1 is an ADC prepared using the VH5VK3 antibody and the TBR site-specific conjugation technology utilizing a linker moiety cleavable by lysosomal cathepsins (‘Val-Cit-PAB’) coupled to the cytotoxic payload MMAE, specifically the linker-drug TBR-Glu-(Val-Cit-PAB-MMAE)-PEG₂₄-Me. This linker format is designed with a branched 24-unit PEG polymer to reduce aggregation propensity and improve aqueous solubility. FIG. 1 shows the structure of the linker reagent which reacts with reduced disulfide bridge groups in the hinge region of the antibody. An average DAR of 4 (i.e. an average of 4 disulfide-bridged linker moieties per antibody) was targeted.

All research batches of TA1 had the appearance of a clear colorless solution with an average DAR of 4 and purity of 95% or higher of monomeric species as determined by size exclusion chromatography (SEC). Endotoxin levels were determined by the EndoSafe®-PTS™ platform (Charles River, Wilmington, MA) aligned with USP <85> and Pharm Eur 2.6.14 that provides quantitative Limulus Amebocyte Lysate (LAL) results. The average molecular weight of TA1 was 158,000 Da as determined by LC-MS. TA1 was handled under sterile conditions and stored at −80° C. until use.

### 1. Test Animals

The present studies were performed using female NSG™ mice implanted with the PDX model TM01141 purchased from Jackson Laboratories. These mice are extremely immunodeficient and carry two mutations: severe combined immune deficiency (scid) and a complete null allele of the IL2 receptor common gamma chain (IL2rg^{null}). The scid mutation is a mutation in the DNA repair complex protein Prkdc and renders the mice B and T cell deficient. The IL2rg^{null} mutation prevents cytokine signaling through multiple receptors, leading to a deficiency in functional NK cells. This severe immunodeficiency allows the implantation of PDX and growth of human tissue-based tumors in the mice. These tumors have similar characteristics to the human tumors from which they are derived (e.g., gene expression) and are predictive of human response to therapeutic agents.

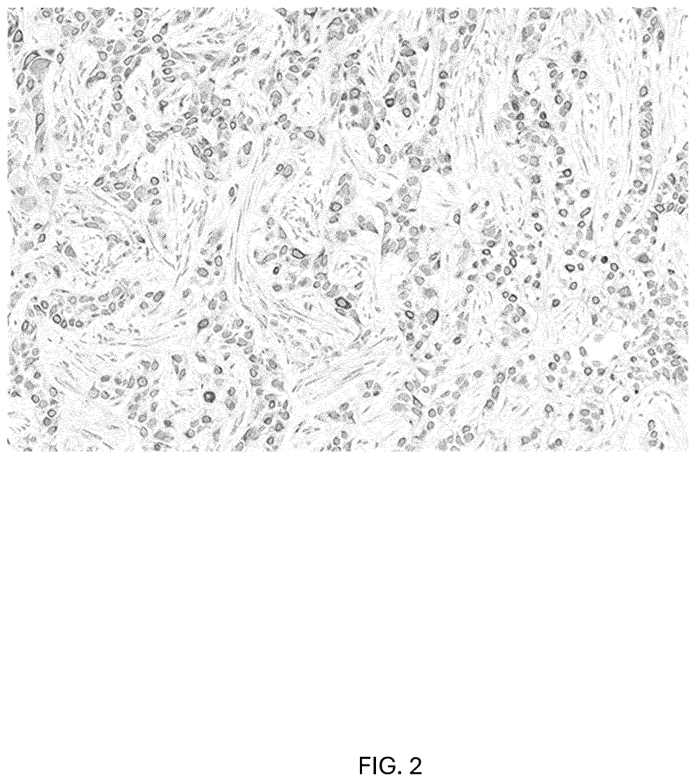

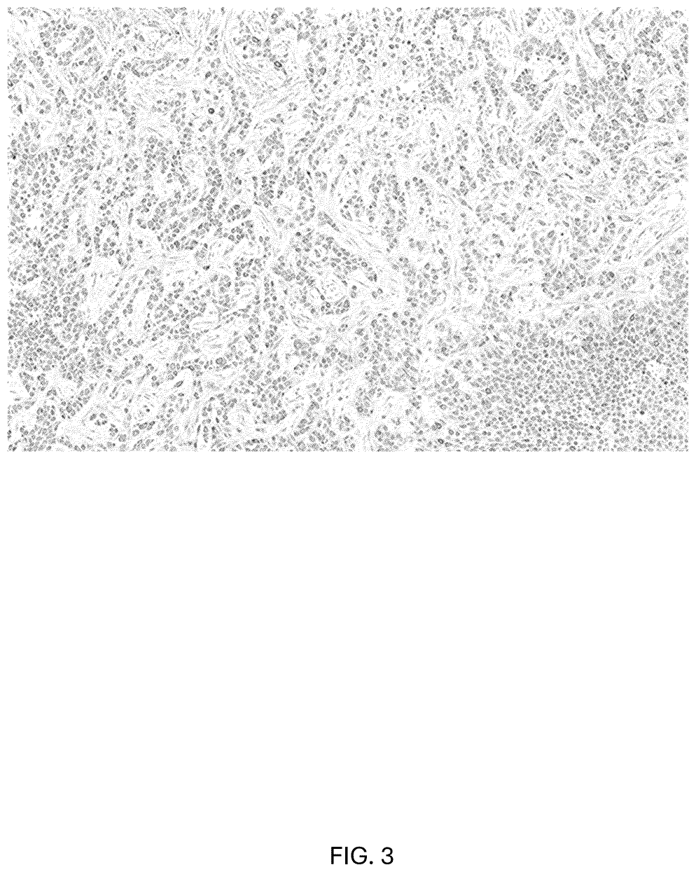

The selection of the PDX model was based on EPHA5 gene expression levels (Jackson Laboratory database) and protein expression by immunohistochemistry (IHC) (See FIGS. 2 and 3). PDX models showing moderate to high EphA5 expression, with a favorable growth curve and deemed responsive to common drugs such as cisplatin, docetaxel and doxorubicin were considered for anti-tumor activity studies. Models resistant to multiple drugs were eliminated from the selection, and models naïve to treatment were prioritized. Based on these criteria, the TM01141 PDX model (JAX Laboratories, Bangor, ME) was selected for the present study.

The initial and final diagnosis of the patient from which the model was derived was squamous cell carcinoma, no grade provided. The primary site of human tumor was the head, and the sample site of the tumor for the PDX model was the primary site of the tumor. The patient from which the sample was derived was a 56-year-old female, with race and ethnicity unreported and unknown if she was a smoker. The sample was obtained by surgical resection. The host strain of the sample for engraftment was NOD.Cg-Prkdc^{scid} Il2rg^{tm1Wjl}/SzJ (aka NSG™ or NOD Scid gamma). Mice were housed in specific pathogen and opportunist-free (SOPF) rooms with controlled temperature (20±2° C.), humidity (50±10%), light/dark cycle (light, 7:00 to 19:00; dark, 19:00 to 7:00), and access to food and water ad libitum at the research animal facilities of the Rutgers Cancer Institute of New Jersey (Newark, NJ). Littermates were randomly assigned to experimental groups. A total of 40 female mice implanted with PDX TM01141 were used in the studies. Mice were received at the study location at 10-12 weeks of age and remained in the study until 18-12 weeks of age on average. Body weights ranged from 22 g to 28 g at the beginning of the studies and 22 g to 28 g at the end of the studies.

### 2. Methods

Mice received 5 weekly doses of TA1 at different doses (2.5 mg/kg, 5 mg/kg and 10 mg/kg) or only the vehicle (PBS) over a 5-week period via tail vein injection. Animals were dosed on days 0, 7, 14, 21 and 28 followed by 3 weeks of observation (day 28-50) without treatment. The number of mice used per group averaged n=8 with some mice presenting with more than 1 tumor implanted subcutaneously. In those cases, each tumor size was measured and recorded independently. Measurements were performed 2-3 times weekly with the aid of a digital caliper (Fisherbrand™ Traceable™ Digital Carbon Fiber Calipers). Studies lasted on average 50 days or until maximum allowed tumor volume was reached and/or skin ulceration was noted. All studies included a control group of an equal number of mice to those in the treatment group. Control groups received PBS tail vein injections and were submitted to the same handling procedures as TA1 treated mice. First treatment was administered when tumors reached approximately 200 mm³ volume.

The body weights of the TM01141-bearing mice enrolled in the studies were measured prior to the first treatment (pre) and at termination. Following termination of the studies, any remaining tumors were excised and weighed on a laboratory balance.

### 3. Data Analysis

The statistical analysis and graphical plotting of the tumor sizes and the body weights was performed using GraphPad Prism 9 and Microsoft Excel (Version 16.63.1). The statistical significance of the measured differences between the test and control groups were tested with Student's t-test or analysis of variance (one-way or two-way ANOVA) using GraphPad Prism 9.

### 4. Results

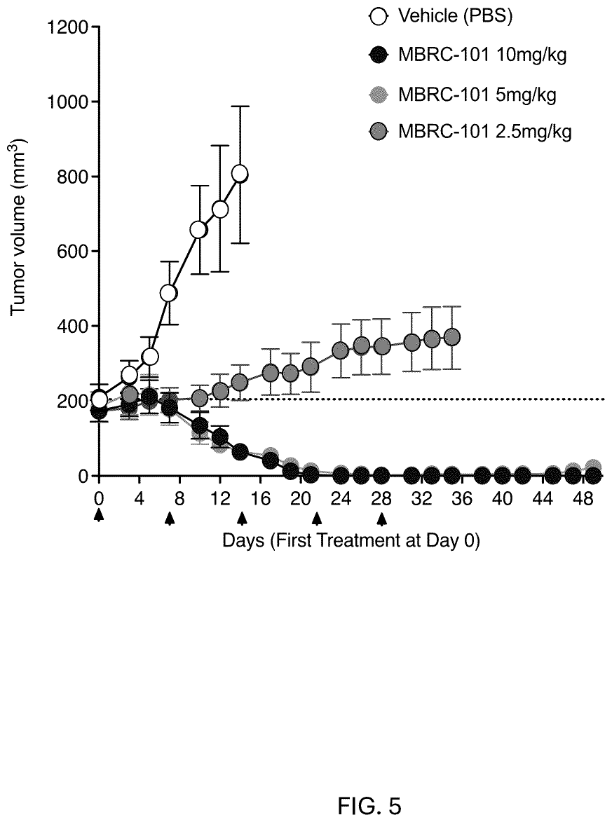

Weekly administration of TA1 to mice bearing TM01141 tumors using different doses of TA-1 (2.5 mg/kg, 5 mg/kg and 10 mg/kg) or only the vehicle (PBS) over a 5-week period resulted in regression of tumors (n=8), with 5 mg·kg and 10 mg·kg displaying sustained regression (FIG. 5).

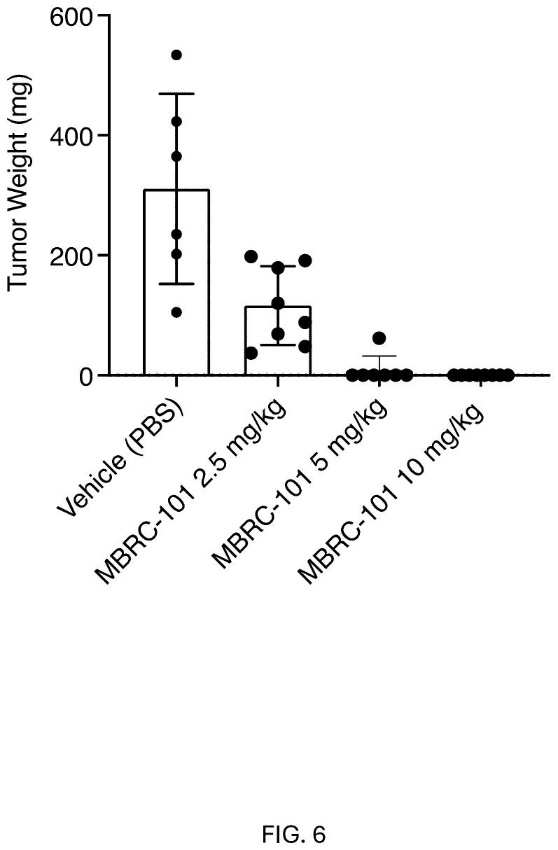

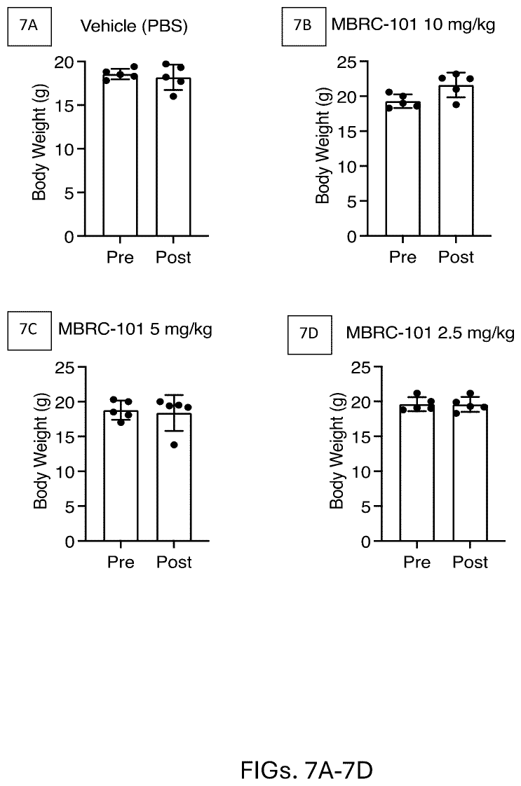

The TA1 treated group 10 mg/kg, the tumors regressed completely, and tumor volume could not be determined at study termination (FIG. 6). In the TA1 treated group receiving 5 mg/kg, all but one treated tumor regressed completely, with the single tumor recovered weighing approximately 0.006 g (FIG. 6). In the TA1 treated group receiving 2.5 mg/kg, eight tumors remained at the end of the study with weights ranging from 0.005 g to 0.021 g, reduced significantly from the tumor weights observed in the placebo treated mice (FIG. 6). As shown in FIGS. 7A-7D, there were no significant changes in body weight of mice in any of the treatment groups before and after treatment with TA1 or placebo.

### Selected Conclusions

TA1 administration to mice bearing the TM01141 PDX model of head and neck SCC over a five-week period resulted in initial regression in the study group receiving 2.5 mg/kg, and sustained regression of tumors in the animals receiving 5 mg/kg and 10 mg/kg during the 50-day study period. Animals treated with TA1 or placebo did not show significant changes in body weight during the study, suggesting the TA1 administration did not have an adverse impact on the animals' ability to thrive. This suggests TA1 is a promising drug candidate for further development for treatment of head and neck SCC.

### Example 2—Synthesis of VH5VK3 TB®-Glu-(Val-Cit-PAB-MMAE)-PEG24-Me

Synthetic examples utilize peptide-peptide and peptide-linker coupling reagents, protecting groups and reactions that are well known to those in the art, see for example, Tarfah I, et al., J. Saudi Chem. Soc., 16, (2), pp. 97-116 (2012).

Some of the linker constructs (L), components of linker constructs, and linker-drug (L-D) constructs are commercially available and can be used in some of the syntheses described below. For example, GMP H-Val-Cit-PAB-MMAE (structure below)

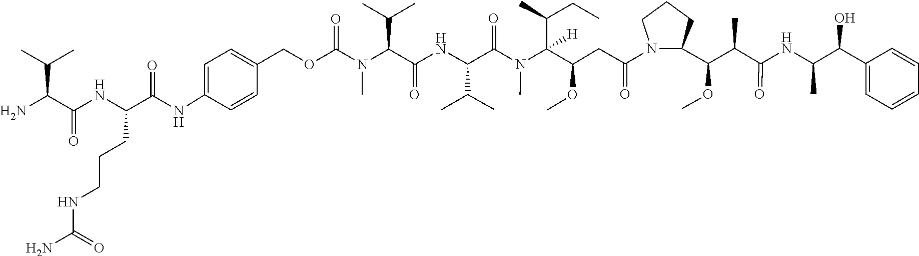 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

is available from MedChemExpress LLC, Princeton NJ, USA (www.medchemexpress.com/).

### Synthesis of TBR-Glu-(Val-Cit-PAB-MMAE)-PEG₂₄-Me

4-[2,2-bis[(p-tolylsulfonyl) methyl]acetyl]benzoic acid-NHS ester was prepared according to Brocchini et al., Nat. Protoc. 1 (2006) 2241-2252. Reaction of EDCI (1-ethyl-3-(3-dimethylaminopropyl)carbodiimide) with the carboxyl group of 4-[2,2-bis[(p-tolylsulfonyl) methyl]acetyl]benzoic acid and reaction with N-hydroxysuccinimide provided the TBR linking reagent as the NHS ester.

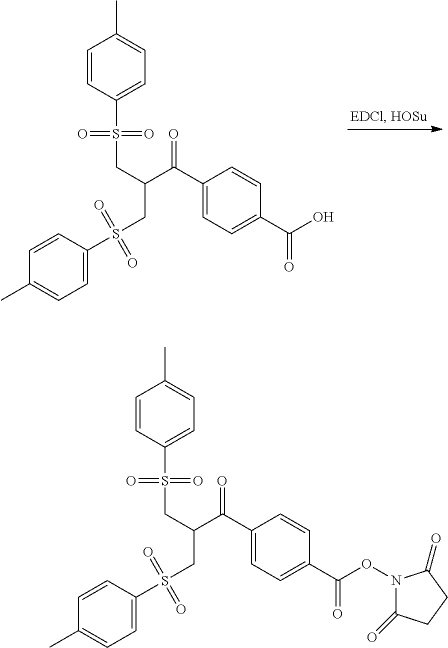 [SMILES: Cc1ccc(S(=O)(=O)CC(CS(=O)(=O)c2ccc(C)cc2)C(=O)c2ccc(C(=O)O)cc2)cc1 ; Cc1ccc(S(=O)(=O)CC(CS(=O)(=O)c2ccc(C)cc2)C(=O)c2ccc(C(=O)ON3C(=O)CCC3=O)cc2)cc1]

4-[2,2-bis[(p-tolylsulfonyl) methyl]acetyl]benzoic acid-NHS ester was reacted with 5-t-butoxy-Glu to give (A).

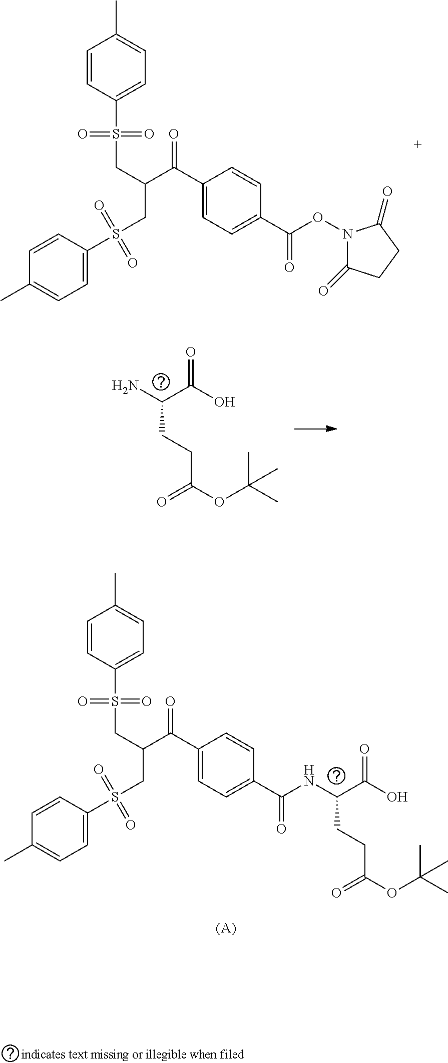 [SMILES: Cc1ccc(S(=O)(=O)CC(CS(=O)(=O)c2ccc(C)cc2)C(=O)c2ccc(C(=O)ON3C(=O)CCC3=O)cc2)cc1 ; CC(C)(C)OC(=O)CC[C@H](N)C(=O)O ; Cc1ccc(S(=O)(=O)CC(CS(=O)(=O)c2ccc(C)cc2)C(=O)c2ccc(C(=O)N[C@@H](CCC(=O)OC(C)(C)C)C(=O)O)cc2)cc1 ; "(A)"]

The intermediate (A) was amidated with H2N-PEG24-Me and subsequently deprotected via TFA hydrolysis. The resulting pegylated free acid intermediate (B) was coupled with Val-Cit-PAB-MMAE using the amide conjugation reagents 1-[Bis(dimethyl-amino) methylene]-1H-1,2,3-triazolo[4,5-b]pyridinium 3-oxide hexafluorophosphate (HA TU) and N-methylmorpholine (NMM) in dimethylformamide (DMF) to form the TB® L-D reagent (C).

As an alternative to HATU and NMM in DMF, the amide conjugation reagents EDCI and HOPO (2-hydroxypyridine-N-oxide) in acetonitrile and water were also used to form (C).

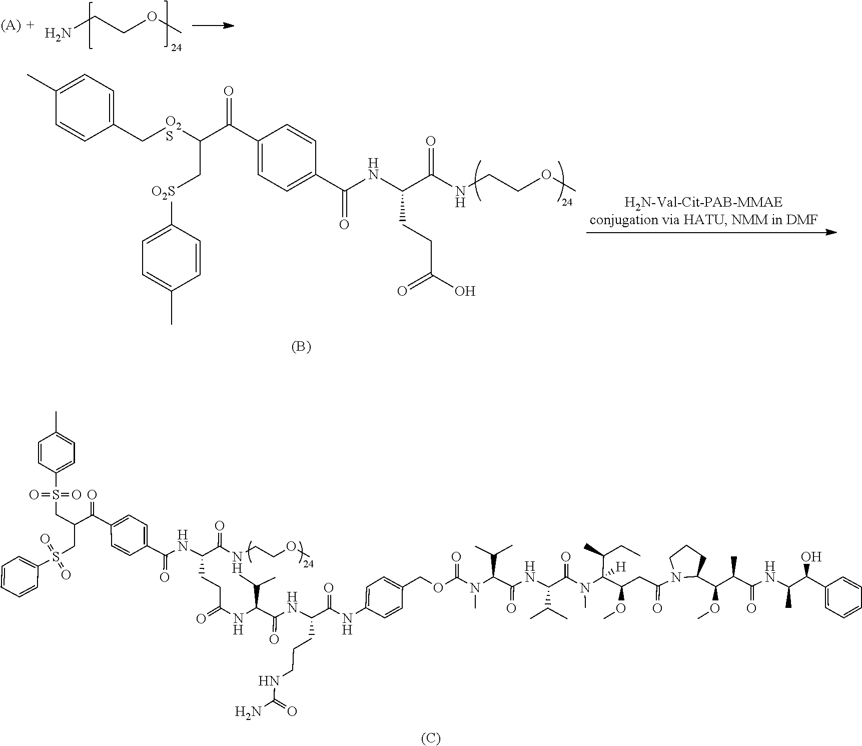

Conjugation of Bis-Sulfone Linker Reagent (C) with Reduced Antibody.

To a solution of antibody at 5 mg/mL in 20 mM sodium phosphate, pH 7.5, 150 mM NaCl and 20 mM EDTA is added a 5 mM TCEP (tris (2-carboxyethyl) phosphine) solution (18 μL) and the resulting mixture is incubated at 40° C. for 1 h. The reduced antibody solution is diluted to 3.33 mg/mL with 20 mM sodium phosphate buffer, pH 7.5, 150 mM NaCl and 20 mM EDTA.

To the obtained reduced VH5VK3 antibody solution, a solution of the bis-sulfone reagent (C) in DMF (at about 3.23 mg/mL) is added and the resulting solution is mixed and incubated at 22° C. for 22 h. The reaction is quenched by addition of 50 mM N-acetyl-L-cysteine (36 μL) and incubated at 22° C. for a further 1 h. The resulting ADC from the quenched conjugation reaction was then purified by hydrophobic interaction chromatography. Reaction time and purification conditions are controlled to achieve an average DAR of 4. Further antibody reduction and bis-sulfone linker conjugation reaction conditions are disclosed in US20210093733 and in “Bridged Cysteine Conjugations.” Methods in molecular biology (Clifton, N.J.) vol. 2078 (2020).

### Example 3: In Vivo Anti-Tumor Activity of ADCs Directed to EphA5 in Two Patient-Derived Xenograft Model of Colorectal Cancer

The objective of this study was to evaluate the therapeutic anti-tumor activity of the ADC MBRC-101 directed to a novel target, EphA5, using two PDX models of colorectal cancer.

### 5. Materials

Test Article 1 (TA1): MBRC-101, as used in Example 1. Test Article 2 (TA2): irinotecan.

### 6. Test Animals

The present studies were performed using female NSG™ mice implanted with the PDX model TM00375 or TM00980 purchased from Jackson Laboratories.

TM00375 was selected due to low EphA5 expression. The diagnosis of the patient from which the model was derived was metastatic colon adenocarcinoma, stage IV. The primary site of human tumor was the colon, and the sample site of the tumor for the PDX model was the muscle metastatic site. The patient from which the sample was derived was a 59-year-old female, with race and ethnicity unreported and smoker status unreported. The sample was obtained by surgical biopsy.

TM00980 was selected due to high EphA5 expression. The diagnosis of the patient from which the model was derived was colon carcinoma, stage IV. The primary site of human tumor was the colon, and the sample site of the tumor for the PDX model was the colon. The patient from which the sample was derived was a 60-year-old male, with race and ethnicity unreported and smoker status unreported. The sample was obtained by an unknown procedure.

### 7. Methods

Mice were treated with weekly injections of 5 mg/kg TA1 and PBS via tail vein injections, or 15 mg/kg irinotecan via intraperitoneal injection as described in Example 1.

### 8. Data Analysis

The statistical analysis and graphical plotting of the tumor sizes and the body weights was performed using GraphPad Prism 9 and Microsoft Excel (Version 16.63.1). The statistical significance of the measured differences between the test and control groups were tested with Student's t-test or analysis of variance (one-way or two-way ANOVA) using GraphPad Prism.

### 9. Results

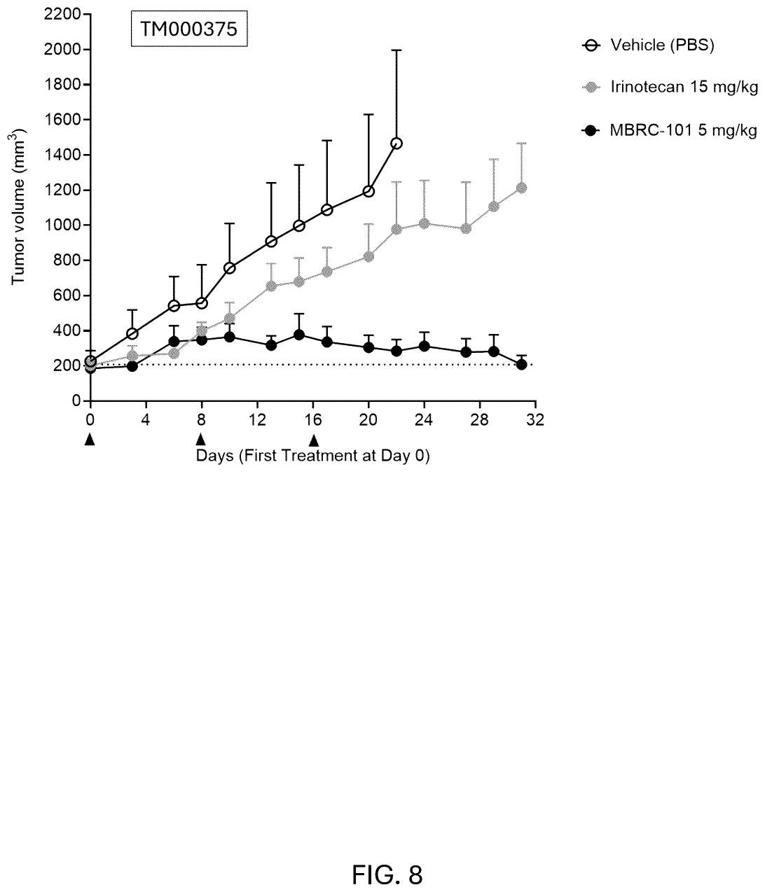

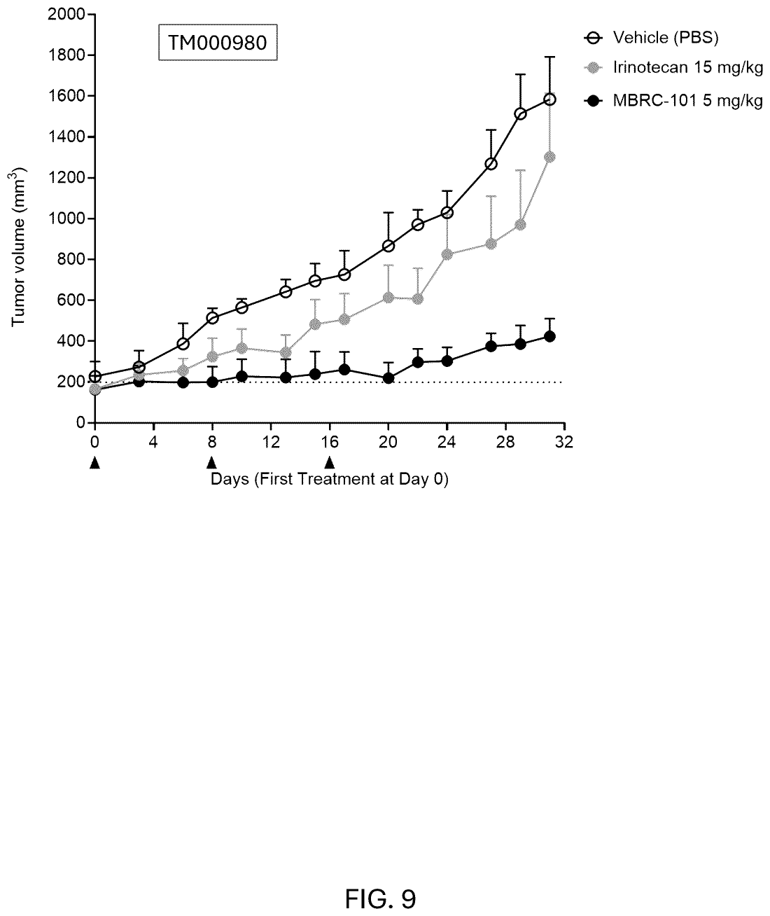

For TM00375, weekly administration of 5 mg/kg TA1 resulted in tumor stasis without complete regressions while weekly administration of 15 mg/kg irinotecan resulted in a slight delay in tumor growth as compared to PBS control (FIG. 8). For TM00980, weekly administration of 5 mg/kg TA1 also resulted in tumor stasis without complete regressions while weekly administration of 15 mg/kg irinotecan resulted in a slight delay in tumor growth as compared to PBS control (FIG. 9). The improvement in anti-tumor activity of TA1 in comparison to irinotecan, a standard of care in the treatment of colorectal cancer, suggests TA1 is a promising drug candidate for further development for treatment of CRC.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 4: In Vivo Anti-Tumor Activity of ADCs Directed to EphA5 in a Patient-Derived Xenograft Model of Pancreatic Cancer

The objective of this study was to evaluate the therapeutic anti-tumor activity of the ADC MBRC-101 in a PDX model of pancreatic cancer.

### 1. Materials

Test Article 1 (TA1): MBRC-101, as used in Example 1. Test Article 2 (TA2): datopotamab deruxtecan, an anti-TROP2 ADC with an average of 4 DXd drugs per antibody.

### 2. Test Animals

The present studies were performed using female NSG™ mice implanted with the PDX model TM01212 purchased from Jackson Laboratories.

TM01212 was derived from a primary tumor site in the pancreas from a male patient.

### 3. Methods

Mice were treated with a single injection of 5 mg/kg TA1 or PBS, or two weekly injections of 10 mg/kg TA2 via tail vein injections as described in Example 1.

### 4. Data Analysis

The statistical analysis and graphical plotting of the tumor sizes and the body weights was performed using GraphPad Prism 9 and Microsoft Excel (Version 16.63.1). The statistical significance of the measured differences between the test and control groups were tested with Student's t-test or analysis of variance (one-way or two-way ANOVA) using GraphPad Prism.

### 5. Results

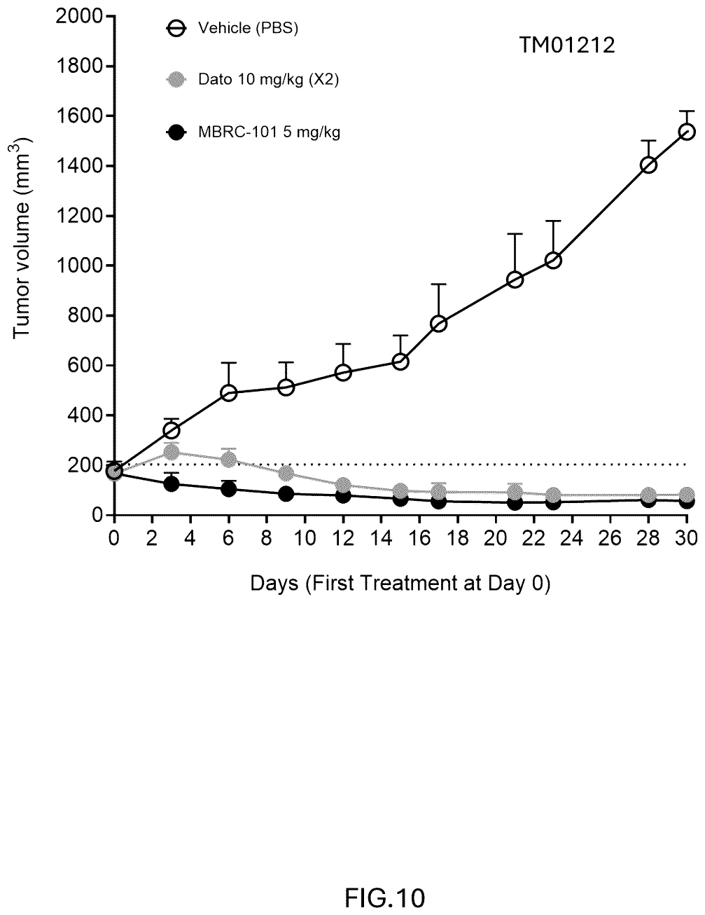

A single administration of 5 mg/kg MBRC-101 resulted in tumor regression with a single complete regression while twice weekly administration of 10 mg/kg datopotamab deruxtecan resulted in tumor regression without complete regression as compared to PBS control (FIG. 10). The anti-tumor activity of TA1 in comparison to TA2, a novel agent being evaluated in multiple cancer types including a recent approval in hormone receptor-positive HER2-negative breast cancer, suggests TA1 is a promising drug candidate for further development for treatment of pancreatic cancer.

### OTHER EMBODIMENTS

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating, ameliorating, and/or preventing squamous cell carcinoma of the head and neck in a subject, the method comprising administering to the subject an effective amount of a composition comprising the components Ab(antibody)-L(linker)-D(drug) as follows: 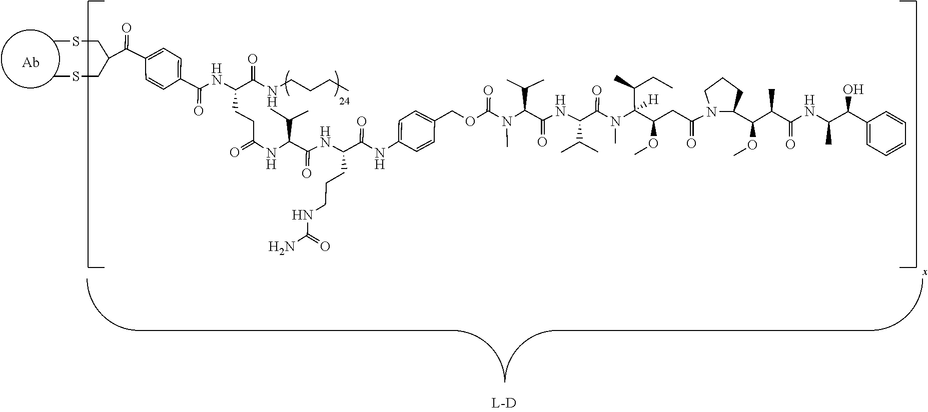
wherein x is an integer from 1 to 8; and
wherein Ab is a human or humanized monoclonal antibody comprising:
a variable heavy chain comprising at least 90% sequence identity to the amino acid chain set forth in SEQ ID NO: 6; and a variable light chain comprising at least 90% sequence identity to the amino acid chain set forth in SEQ ID NO: 11.

2. The method of claim 1, wherein said variable heavy chain comprises the amino acid chain set forth in SEQ ID NO: 6; and wherein said variable light chain comprises the amino acid chain set forth in SEQ ID NO: 11.

3. The method of claim 1, wherein the composition is administered by injection or infusion at a dose of 0.5 or more mg/kg, 1.0 or more mg/kg, 2.0 or more mg/kg, 4.0 or more mg/kg, or 10.0 or more mg/kg.

4. The method of claim 1, wherein the composition is administered by injection or infusion twice a week, three time a week, weekly, bi-weekly, every three weeks, or monthly.

5. A method for treating, ameliorating, and/or preventing squamous cell carcinoma of the head and neck in a subject, the method comprising administering to the subject an effective amount of a composition comprising the components Ab(antibody)-L(linker)-D(drug) as follows: 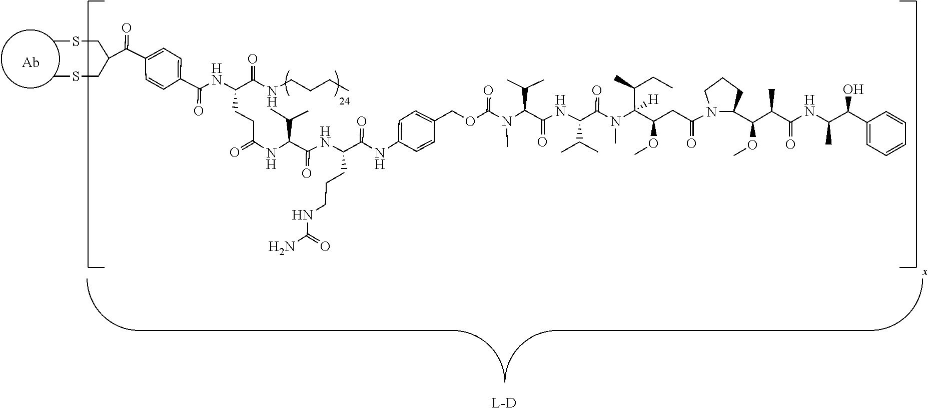
wherein x is an integer from 1 to 8; and
wherein Ab is a human or humanized monoclonal antibody comprising:
(a) a first VH CDR comprising at least 90% sequence identity to the amino acid chain set forth in SEQ ID NO: 16;
(b) a second VH CDR comprising at least 90% sequence identity to the amino acid chain set forth in SEQ ID NO: 18;
(c) a third VH CDR comprising at least 90% sequence identity to the amino acid chain set forth in SEQ ID NO: 20;
(d) a first VL CDR comprising at least 90% sequence identity to the amino acid chain set forth in SEQ ID NO: 21;
(e) a second VL CDR comprising at least 90% sequence identity to the amino acid chain set forth in SEQ ID NO: 23; and
(f) a third VL CDR comprising at least 90% sequence identity to the amino acid chain set forth in SEQ ID NO: 24.

6. The method of claim 5, wherein said first VH CDR comprises the amino acid chain set forth in SEQ ID NO: 16; said second VH CDR comprises the amino acid chain set forth in SEQ ID NO: 18; said third VH CDR comprises the amino acid chain set forth in SEQ ID NO: 20; said first VL CDR comprises the amino acid chain set forth in SEQ ID NO: 21; said second VL CDR comprises the amino acid chain set forth in SEQ ID NO: 23; and said third VL CDR comprises the amino acid chain set forth in SEQ ID NO: 24.

7. The method of claim 5, wherein the composition is administered by injection or infusion at a dose of 0.5 or more mg/kg, 1.0 or more mg/kg, 2.0 or more mg/kg, 4.0 or more mg/kg, or 10.0 or more mg/kg.

8. The method of claim 5, wherein the composition is administered by injection or infusion twice a week, three time a week, weekly, bi-weekly, every three weeks, or monthly.

9. A method for treating, ameliorating, and/or preventing squamous cell carcinoma of the head and neck in a subject, the method comprising administering to the subject an effective amount of a composition comprising the components Ab(antibody)-L(linker)-D(drug) as follows: 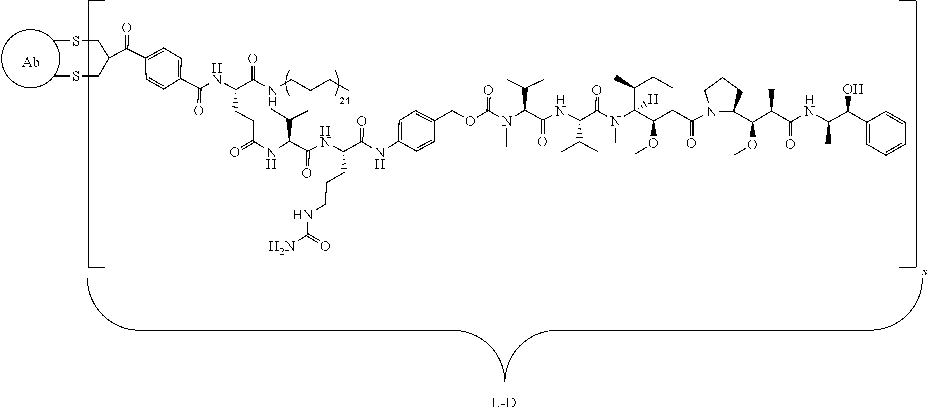
wherein x is an integer from 1 to 8; and
wherein Ab is a human or humanized monoclonal antibody comprising:
a variable heavy chain comprising at least 90% sequence identity to the amino acid chain set forth in SEQ ID NO: 39; and a variable light chain comprising at least 90% sequence identity to the amino acid chain set forth in SEQ ID NO: 40.

10. The method of claim 9, wherein said variable heavy chain comprises the amino acid chain set forth in SEQ ID NO: 39; and wherein said variable light chain comprises the amino acid chain set forth in SEQ ID NO: 40.

11. The method of claim 9, wherein the composition is administered by injection or infusion at a dose of 0.5 or more mg/kg, 1.0 or more mg/kg, 2.0 or more mg/kg, 4.0 or more mg/kg, or 10.0 or more mg/kg.

12. The method of claim 9, wherein the composition is administered by injection or infusion twice a week, three time a week, weekly, bi-weekly, every three weeks, or monthly.

13. A pharmaceutical composition for use in treating, ameliorating, and/or preventing squamous cell carcinoma of the head and neck in a subject in need thereof, wherein the cancer is associated with expression of EPH Receptor A5, and the composition comprises the components Ab(antibody)-L(linker)-D(drug) as follows: 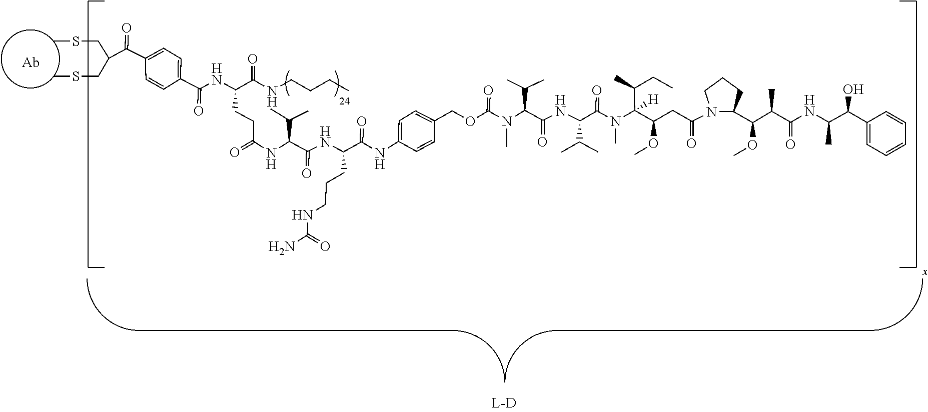
wherein the L-D portion of the composition has an x is from 1 to 8; and
wherein the Ab component is a human or humanized monoclonal antibody comprising:
a variable heavy chain comprising at least 90% sequence identity to the amino acid chain set forth in SEQ ID NO: 6; and a variable light chain comprising at least 90% sequence identity to the amino acid chain set forth in SEQ ID NO: 11.

14. The pharmaceutical composition of claim 13, wherein said variable heavy chain comprises the amino acid chain set forth in SEQ ID NO: 6; and wherein said variable light chain comprises the amino acid chain set forth in SEQ ID NO: 11.

15. The pharmaceutical composition of claim 13, wherein the composition is formulated to dose by injection or infusion at 0.5 or more mg/kg, 1.0 or more mg/kg, 2.0 or more mg/kg, 4.0 or more mg/kg, or 10.0 or more mg/kg.

16. The pharmaceutical composition of claim 13, wherein the composition is formulated for administration twice a week, three time a week, weekly, bi-weekly, every three weeks, or monthly.

17. A pharmaceutical composition for use in treating, ameliorating, and/or preventing squamous cell carcinoma of the head and neck in a subject in need thereof, wherein the cancer is associated with expression of EPH Receptor A5, and the composition comprises the components Ab(antibody)-L(linker)-D(drug) as follows: 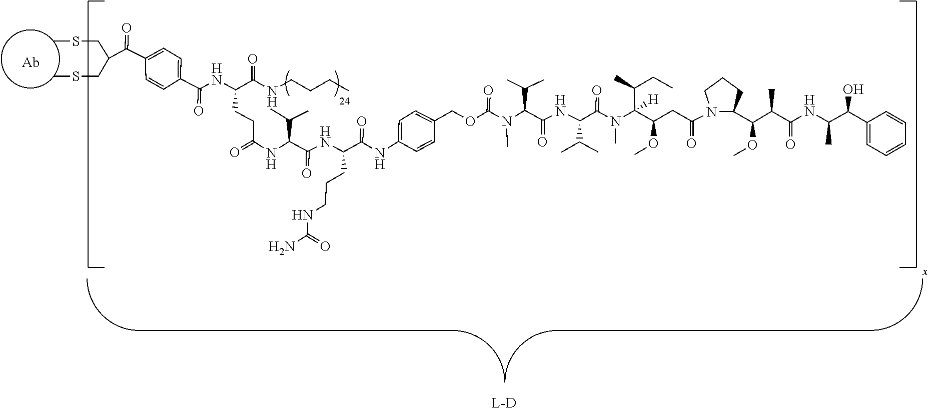
wherein the L-D portion of the composition has an x is from 1 to 8; and
wherein the Ab component is a human or humanized monoclonal antibody comprising:
a variable heavy chain comprising at least 90% sequence identity to the amino acid chain set forth in SEQ ID NO: 39; and a variable light chain comprising at least 90% sequence identity to the amino acid chain set forth in SEQ ID NO: 40.

18. The pharmaceutical composition of claim 17, wherein said variable heavy chain comprises the amino acid chain set forth in SEQ ID NO: 39; and wherein said variable light chain comprises the amino acid chain set forth in SEQ ID NO: 40.

19. The pharmaceutical composition of claim 17, wherein the composition is formulated to dose by injection or infusion at 0.5 or more mg/kg, 1.0 or more mg/kg, 2.0 or more mg/kg, 4.0 or more mg/kg, or 10.0 or more mg/kg.

20. The pharmaceutical composition of claim 17, wherein the composition is formulated for administration twice a week, three time a week, weekly, bi-weekly, every three weeks, or monthly.

21. A pharmaceutical composition for use in treating, ameliorating, and/or preventing squamous cell carcinoma of the head and neck in a subject in need thereof, wherein the cancer is associated with expression of EPH Receptor A5, and the composition comprises the components Ab(antibody)-L(linker)-D(drug) as follows: 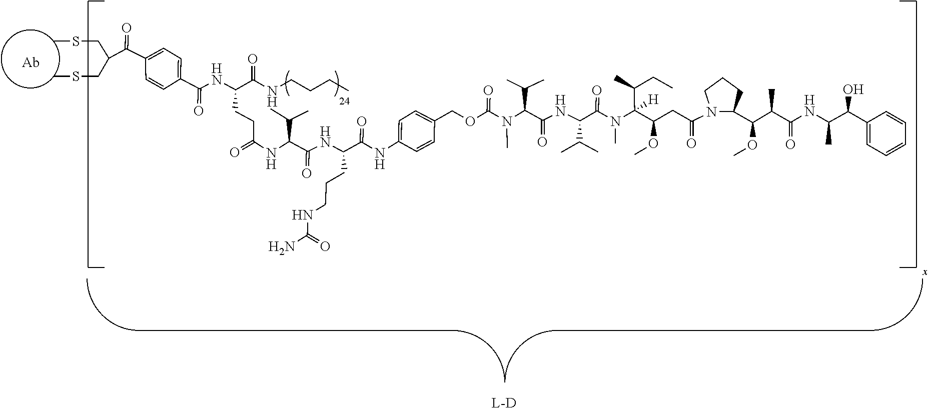
wherein the L-D portion of the composition has an x is from 1 to 8; and
wherein the Ab component is a human or humanized monoclonal antibody comprising:
(a) a first VH CDR comprising at least 90% sequence identity to the amino acid chain set forth in SEQ ID NO: 16;
(b) a second VH CDR comprising at least 90% sequence identity to the amino acid chain set forth in SEQ ID NO: 18;
(c) a third VH CDR comprising at least 90% sequence identity to the amino acid chain set forth in SEQ ID NO: 20;
(d) a first VL CDR comprising at least 90% sequence identity to the amino acid chain set forth in SEQ ID NO: 21;
(e) a second VL CDR comprising at least 90% sequence identity to the amino acid chain set forth in SEQ ID NO: 23; and
(f) a third VL CDR comprising at least 90% sequence identity to the amino acid chain set forth in SEQ ID NO: 24.

22. The pharmaceutical composition of claim 21, wherein said first VH CDR comprises the amino acid chain set forth in SEQ ID NO: 16; said second VH CDR comprises the amino acid chain set forth in SEQ ID NO: 18; said third VH CDR comprises the amino acid chain set forth in SEQ ID NO: 20; said first VL CDR comprises the amino acid chain set forth in SEQ ID NO: 21; said second VL CDR comprises the amino acid chain set forth in SEQ ID NO: 23; and said third VL CDR comprises the amino acid chain set forth in SEQ ID NO: 24.

23. The pharmaceutical composition of claim 21, wherein the composition is formulated to dose by injection or infusion at 0.5 or more mg/kg, 1.0 or more mg/kg, 2.0 or more mg/kg, 4.0 or more mg/kg, or 10.0 or more mg/kg.

24. The pharmaceutical composition of claim 21, wherein the composition is formulated for administration twice a week, three time a week, weekly, bi-weekly, every three weeks, or monthly.

25. A method for treating, ameliorating, and/or preventing colorectal cancer in a subject, the method comprising administering to the subject an effective amount of a composition comprising the components Ab(antibody)-L(linker)-D(drug) as follows: 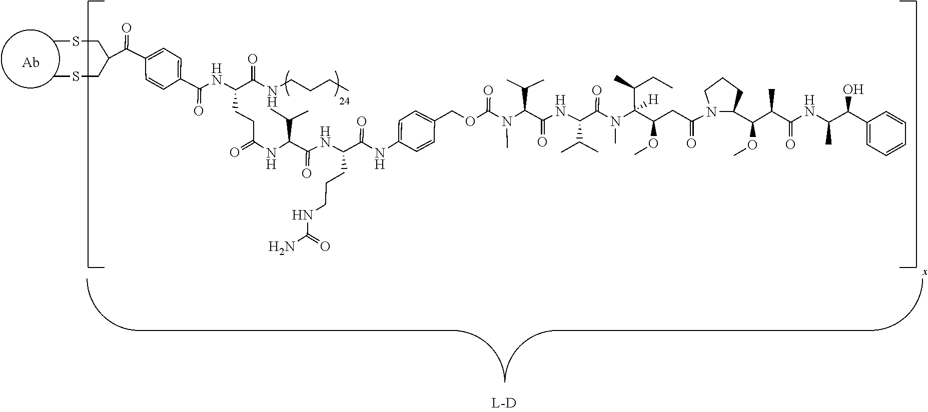
wherein x is an integer from 1 to 8; and
wherein Ab is a human or humanized monoclonal antibody comprising:
a variable heavy chain comprising at least 90% sequence identity to the amino acid chain set forth in SEQ ID NO: 6; and a variable light chain comprising at least 90% sequence identity to the amino acid chain set forth in SEQ ID NO: 11.

26. The method of claim 25, wherein said variable heavy chain comprises the amino acid chain set forth in SEQ ID NO: 6; and wherein said variable light chain comprises the amino acid chain set forth in SEQ ID NO: 11.

27. The method of claim 25, wherein the composition is administered by injection or infusion at a dose of 0.5 or more mg/kg, 1.0 or more mg/kg, 2.0 or more mg/kg, 4.0 or more mg/kg, or 10.0 or more mg/kg.

28. The method of claim 25, wherein the composition is administered by injection or infusion twice a week, three time a week, weekly, bi-weekly, every three weeks, or monthly.

29. A method for treating, ameliorating, and/or preventing colorectal cancer in a subject, the method comprising administering to the subject an effective amount of a composition comprising the components Ab(antibody)-L(linker)-D(drug) as follows: 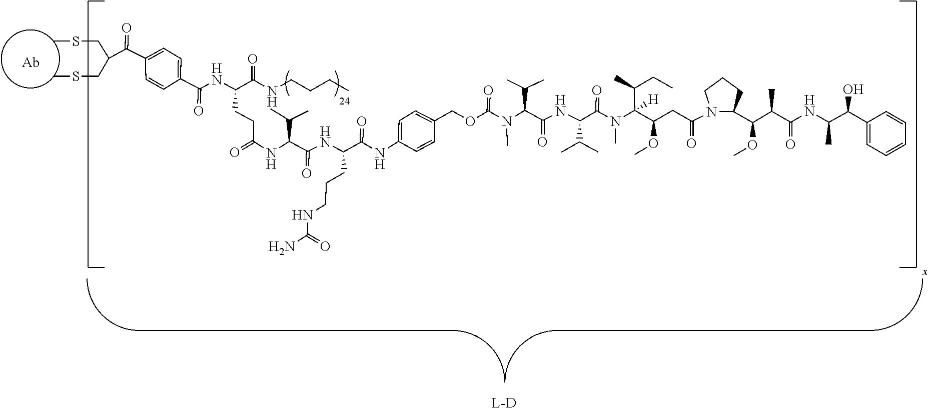
wherein x is an integer from 1 to 8; and
wherein Ab is a human or humanized monoclonal antibody comprising:
(a) a first VH CDR comprising at least 90% sequence identity to the amino acid chain set forth in SEQ ID NO: 16;
(b) a second VH CDR comprising at least 90% sequence identity to the amino acid chain set forth in SEQ ID NO: 18;
(c) a third VH CDR comprising at least 90% sequence identity to the amino acid chain set forth in SEQ ID NO: 20;
(d) a first VL CDR comprising at least 90% sequence identity to the amino acid chain set forth in SEQ ID NO: 21;
(e) a second VL CDR comprising at least 90% sequence identity to the amino acid chain set forth in SEQ ID NO: 23; and
(f) a third VL CDR comprising at least 90% sequence identity to the amino acid chain set forth in SEQ ID NO: 24.

30. The method of claim 29, wherein said first VH CDR comprises the amino acid chain set forth in SEQ ID NO: 16; said second VH CDR comprises the amino acid chain set forth in SEQ ID NO: 18; said third VH CDR comprises the amino acid chain set forth in SEQ ID NO: 20; said first VL CDR comprises the amino acid chain set forth in SEQ ID NO: 21; said second VL CDR comprises the amino acid chain set forth in SEQ ID NO: 23; and said third VL CDR comprises the amino acid chain set forth in SEQ ID NO: 24.

31. The method of claim 29, wherein the composition is administered by injection or infusion at a dose of 0.5 or more mg/kg, 1.0 or more mg/kg, 2.0 or more mg/kg, 4.0 or more mg/kg, or 10.0 or more mg/kg.

32. The method of claim 29, wherein the composition is administered by injection or infusion twice a week, three time a week, weekly, bi-weekly, every three weeks, or monthly.

33. A method for treating, ameliorating, and/or preventing colorectal cancer in a subject, the method comprising administering to the subject an effective amount of a composition comprising the components Ab(antibody)-L(linker)-D(drug) as follows: 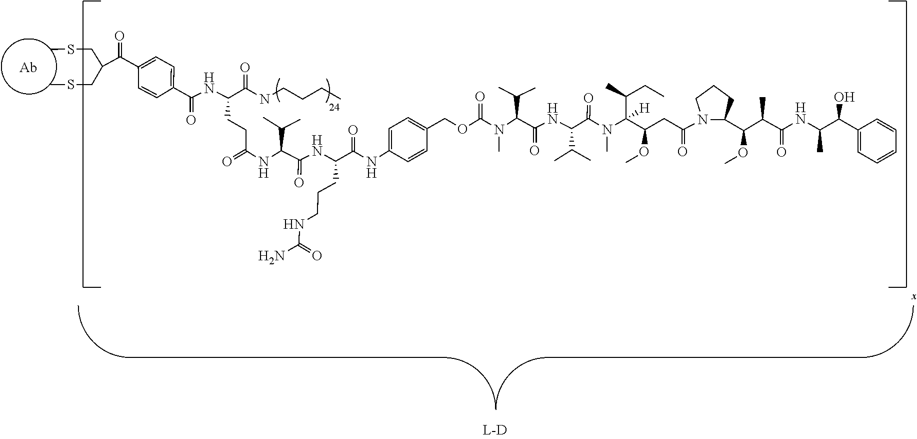
wherein x is an integer from 1 to 8; and
wherein Ab is a human or humanized monoclonal antibody comprising:
a variable heavy chain comprising at least 90% sequence identity to the amino acid chain set forth in SEQ ID NO: 39; and a variable light chain comprising at least 90% sequence identity to the amino acid chain set forth in SEQ ID NO: 40.

34. The method of claim 33, wherein said variable heavy chain comprises the amino acid chain set forth in SEQ ID NO: 39; and wherein said variable light chain comprises the amino acid chain set forth in SEQ ID NO: 40.

35. The method of claim 33, wherein the composition is administered by injection or infusion at a dose of 0.5 or more mg/kg, 1.0 or more mg/kg, 2.0 or more mg/kg, 4.0 or more mg/kg, or 10.0 or more mg/kg.

36. The method of claim 33, wherein the composition is administered by injection or infusion twice a week, three time a week, weekly, bi-weekly, every three weeks, or monthly.

37. A pharmaceutical composition for use in treating, ameliorating, and/or preventing colorectal cancer in a subject in need thereof, wherein the cancer is associated with expression of EPH Receptor A5, and the composition comprises the components Ab(antibody)-L(linker)-D(drug) as follows: 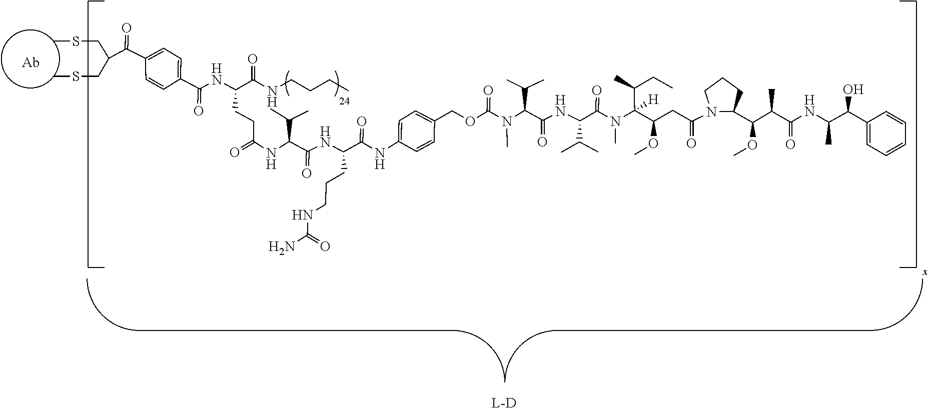
wherein the L-D portion of the composition has an x is from 1 to 8; and
wherein the Ab component is a human or humanized monoclonal antibody comprising:
a variable heavy chain comprising at least 90% sequence identity to the amino acid chain set forth in SEQ ID NO: 6; and a variable light chain comprising at least 90% sequence identity to the amino acid chain set forth in SEQ ID NO: 11.

38. The pharmaceutical composition of claim 37, wherein said variable heavy chain comprises the amino acid chain set forth in SEQ ID NO: 6; and wherein said variable light chain comprises the amino acid chain set forth in SEQ ID NO: 11.

39. The pharmaceutical composition of claim 37, wherein the composition is formulated to dose by injection or infusion at 0.5 or more mg/kg, 1.0 or more mg/kg, 2.0 or more mg/kg, 4.0 or more mg/kg, or 10.0 or more mg/kg.

40. The pharmaceutical composition of claim 37, wherein the composition is formulated for administration twice a week, three time a week, weekly, bi-weekly, every three weeks, or monthly.

41. A pharmaceutical composition for use in treating, ameliorating, and/or preventing colorectal cancer in a subject in need thereof, wherein the cancer is associated with expression of EPH Receptor A5, and the composition comprises the components Ab(antibody)-L(linker)-D(drug) as follows: 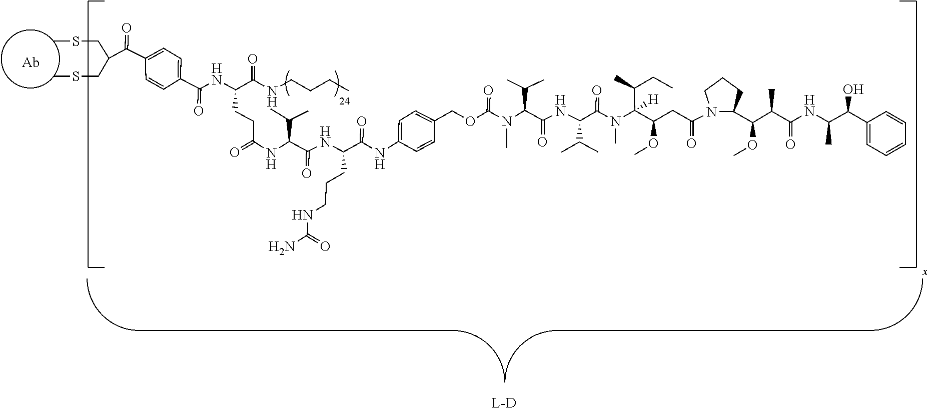
wherein the L-D portion of the composition has an x is from 1 to 8; and
wherein the Ab component is a human or humanized monoclonal antibody comprising:
a variable heavy chain comprising at least 90% sequence identity to the amino acid chain set forth in SEQ ID NO: 39; and a variable light chain comprising at least 90% sequence identity to the amino acid chain set forth in SEQ ID NO: 40.

42. The pharmaceutical composition of claim 41, wherein said variable heavy chain comprises the amino acid chain set forth in SEQ ID NO: 39; and wherein said variable light chain comprises the amino acid chain set forth in SEQ ID NO: 40.

43. The pharmaceutical composition of claim 41, wherein the composition is formulated to dose by injection or infusion at 0.5 or more mg/kg, 1.0 or more mg/kg, 2.0 or more mg/kg, 4.0 or more mg/kg, or 10.0 or more mg/kg.

44. The pharmaceutical composition of claim 41, wherein the composition is formulated for administration twice a week, three time a week, weekly, bi-weekly, every three weeks, or monthly.

45. A pharmaceutical composition for use in treating, ameliorating, and/or preventing colorectal cancer in a subject in need thereof, wherein the cancer is associated with expression of EPH Receptor A5, and the composition comprises the components Ab(antibody)-L(linker)-D(drug) as follows: 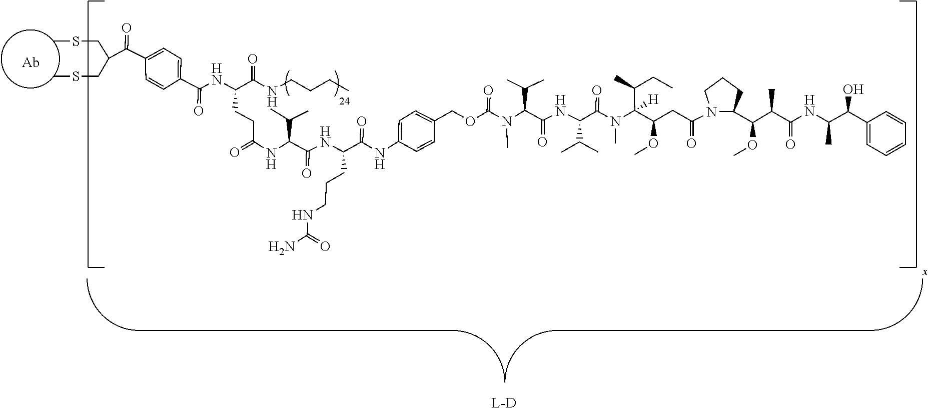
wherein the L-D portion of the composition has an x is from 1 to 8; and
wherein the Ab component is a human or humanized monoclonal antibody comprising:
(a) a first VH CDR comprising at least 90% sequence identity to the amino acid chain set forth in SEQ ID NO: 16;
(b) a second VH CDR comprising at least 90% sequence identity to the amino acid chain set forth in SEQ ID NO: 18;
(c) a third VH CDR comprising at least 90% sequence identity to the amino acid chain set forth in SEQ ID NO: 20;
(d) a first VL CDR comprising at least 90% sequence identity to the amino acid chain set forth in SEQ ID NO: 21;
(e) a second VL CDR comprising at least 90% sequence identity to the amino acid chain set forth in SEQ ID NO: 23; and
(f) a third VL CDR comprising at least 90% sequence identity to the amino acid chain set forth in SEQ ID NO: 24.

46. The pharmaceutical composition of claim 45, wherein said first VH CDR comprises the amino acid chain set forth in SEQ ID NO: 16; said second VH CDR comprises the amino acid chain set forth in SEQ ID NO: 18; said third VH CDR comprises the amino acid chain set forth in SEQ ID NO: 20; said first VL CDR comprises the amino acid chain set forth in SEQ ID NO: 21; said second VL CDR comprises the amino acid chain set forth in SEQ ID NO: 23; and said third VL CDR comprises the amino acid chain set forth in SEQ ID NO: 24.

47. The pharmaceutical composition of claim 45, wherein the composition is formulated to dose by injection or infusion at 0.5 or more mg/kg, 1.0 or more mg/kg, 2.0 or more mg/kg, 4.0 or more mg/kg, or 10.0 or more mg/kg.

48. The pharmaceutical composition of claim 45, wherein the composition is formulated for administration twice a week, three time a week, weekly, bi-weekly, every three weeks, or monthly.

49. A method for treating, ameliorating, and/or preventing pancreatic cancer in a subject, the method comprising administering to the subject an effective amount of a composition comprising the components Ab(antibody)-L(linker)-D(drug) as follows: 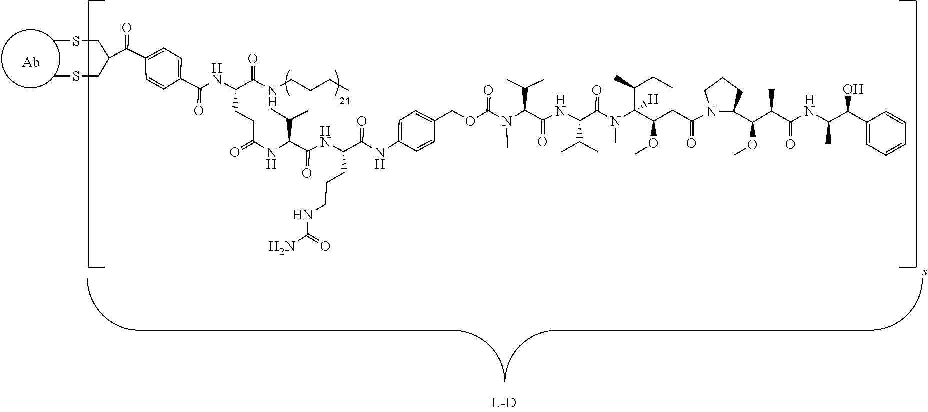
wherein x is an integer from 1 to 8; and
wherein Ab is a human or humanized monoclonal antibody comprising:
a variable heavy chain comprising at least 90% sequence identity to the amino acid chain set forth in SEQ ID NO: 6; and a variable light chain comprising at least 90% sequence identity to the amino acid chain set forth in SEQ ID NO: 11.

50. The method of claim 49, wherein said variable heavy chain comprises the amino acid chain set forth in SEQ ID NO: 6; and wherein said variable light chain comprises the amino acid chain set forth in SEQ ID NO: 11.

51. The method of claim 49, wherein the composition is administered by injection or infusion at a dose of 0.5 or more mg/kg, 1.0 or more mg/kg, 2.0 or more mg/kg, 4.0 or more mg/kg, or 10.0 or more mg/kg.

52. The method of claim 49, wherein the composition is administered by injection or infusion twice a week, three time a week, weekly, bi-weekly, every three weeks, or monthly.

53. A method for treating, ameliorating, and/or preventing pancreatic cancer in a subject, the method comprising administering to the subject an effective amount of a composition comprising the components Ab(antibody)-L(linker)-D(drug) as follows: 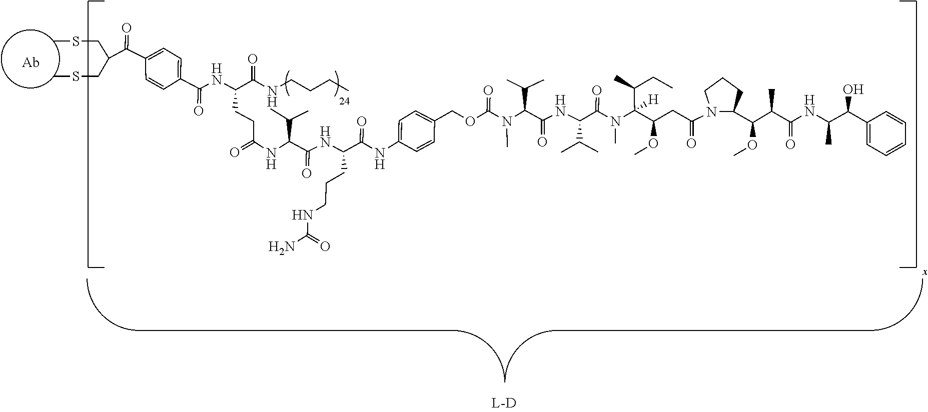
wherein x is an integer from 1 to 8; and
wherein Ab is a human or humanized monoclonal antibody comprising:
(a) a first VH CDR comprising at least 90% sequence identity to the amino acid chain set forth in SEQ ID NO: 16;
(b) a second VH CDR comprising at least 90% sequence identity to the amino acid chain set forth in SEQ ID NO: 18;
(c) a third VH CDR comprising at least 90% sequence identity to the amino acid chain set forth in SEQ ID NO: 20;
(d) a first VL CDR comprising at least 90% sequence identity to the amino acid chain set forth in SEQ ID NO: 21;
(e) a second VL CDR comprising at least 90% sequence identity to the amino acid chain set forth in SEQ ID NO: 23; and
(f) a third VL CDR comprising at least 90% sequence identity to the amino acid chain set forth in SEQ ID NO: 24.

54. The method of claim 53, wherein said first VH CDR comprises the amino acid chain set forth in SEQ ID NO: 16; said second VH CDR comprises the amino acid chain set forth in SEQ ID NO: 18; said third VH CDR comprises the amino acid chain set forth in SEQ ID NO: 20; said first VL CDR comprises the amino acid chain set forth in SEQ ID NO: 21; said second VL CDR comprises the amino acid chain set forth in SEQ ID NO: 23; and said third VL CDR comprises the amino acid chain set forth in SEQ ID NO: 24.

55. The method of claim 53, wherein the composition is administered by injection or infusion at a dose of 0.5 or more mg/kg, 1.0 or more mg/kg, 2.0 or more mg/kg, 4.0 or more mg/kg, or 10.0 or more mg/kg.

56. The method of claim 53, wherein the composition is administered by injection or infusion twice a week, three time a week, weekly, bi-weekly, every three weeks, or monthly.

57. A method for treating, ameliorating, and/or preventing pancreatic cancer in a subject, the method comprising administering to the subject an effective amount of a composition comprising the components Ab(antibody)-L(linker)-D(drug) as follows: 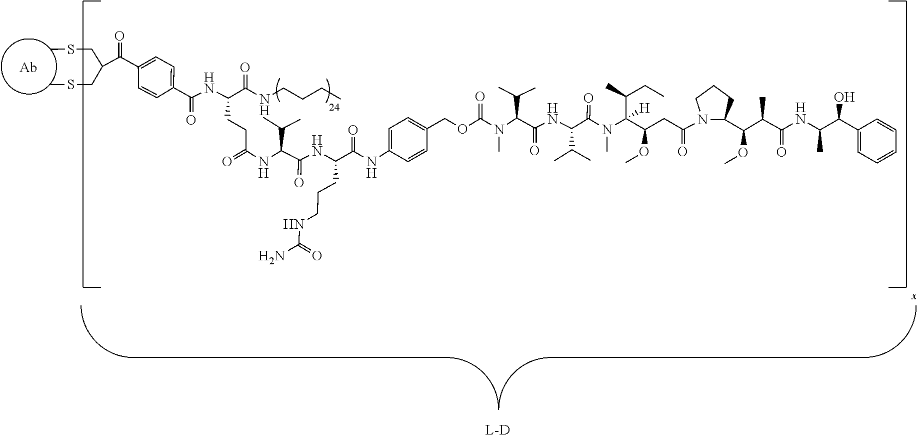
wherein x is an integer from 1 to 8; and
wherein Ab is a human or humanized monoclonal antibody comprising:
a variable heavy chain comprising at least 90% sequence identity to the amino acid chain set forth in SEQ ID NO: 39; and a variable light chain comprising at least 90% sequence identity to the amino acid chain set forth in SEQ ID NO: 40.

58. The method of claim 57, wherein said variable heavy chain comprises the amino acid chain set forth in SEQ ID NO: 39; and wherein said variable light chain comprises the amino acid chain set forth in SEQ ID NO: 40.

59. The method of claim 57, wherein the composition is administered by injection or infusion at a dose of 0.5 or more mg/kg, 1.0 or more mg/kg, 2.0 or more mg/kg, 4.0 or more mg/kg, or 10.0 or more mg/kg.

60. The method of claim 57, wherein the composition is administered by injection or infusion twice a week, three time a week, weekly, bi-weekly, every three weeks, or monthly.

61. A pharmaceutical composition for use in treating, ameliorating, and/or preventing pancreatic cancer in a subject in need thereof, wherein the cancer is associated with expression of EPH Receptor A5, and the composition comprises the components Ab(antibody)-L(linker)-D(drug) as follows: 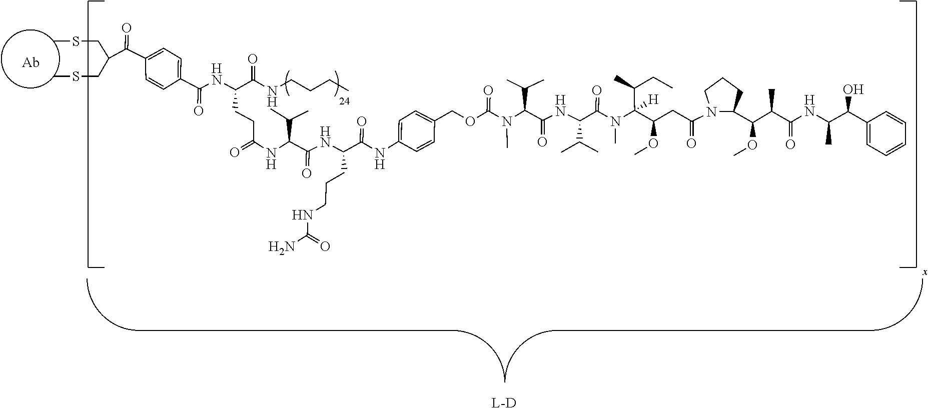
wherein the L-D portion of the composition has an x is from 1 to 8; and
wherein the Ab component is a human or humanized monoclonal antibody comprising:
a variable heavy chain comprising at least 90% sequence identity to the amino acid chain set forth in SEQ ID NO: 6; and a variable light chain comprising at least 90% sequence identity to the amino acid chain set forth in SEQ ID NO: 11.

62. The pharmaceutical composition of claim 61, wherein said variable heavy chain comprises the amino acid chain set forth in SEQ ID NO: 6; and wherein said variable light chain comprises the amino acid chain set forth in SEQ ID NO: 11.

63. The pharmaceutical composition of claim 61, wherein the composition is formulated to dose by injection or infusion at 0.5 or more mg/kg, 1.0 or more mg/kg, 2.0 or more mg/kg, 4.0 or more mg/kg, or 10.0 or more mg/kg.

64. The pharmaceutical composition of claim 61, wherein the composition is formulated for administration twice a week, three time a week, weekly, bi-weekly, every three weeks, or monthly.

65. A pharmaceutical composition for use in treating, ameliorating, and/or preventing pancreatic cancer in a subject in need thereof, wherein the cancer is associated with expression of EPH Receptor A5, and the composition comprises the components Ab(antibody)-L(linker)-D(drug) as follows: 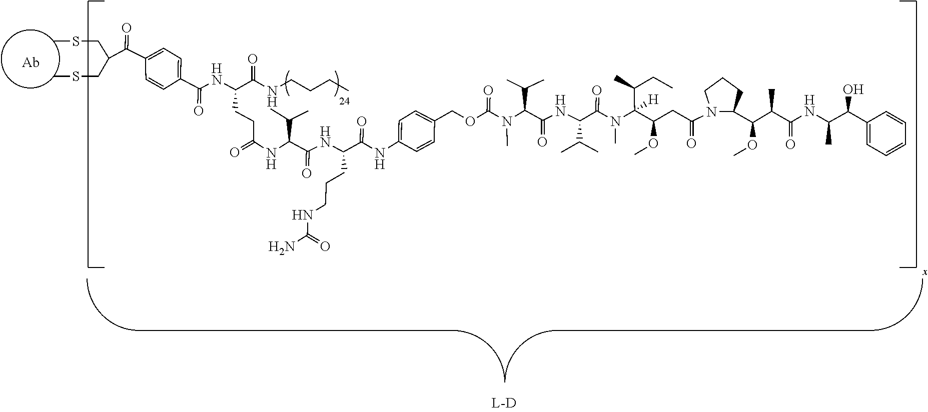
wherein the L-D portion of the composition has an x is from 1 to 8; and
wherein the Ab component is a human or humanized monoclonal antibody comprising:
a variable heavy chain comprising at least 90% sequence identity to the amino acid chain set forth in SEQ ID NO: 39; and a variable light chain comprising at least 90% sequence identity to the amino acid chain set forth in SEQ ID NO: 40.

66. The pharmaceutical composition of claim 65, wherein said variable heavy chain comprises the amino acid chain set forth in SEQ ID NO: 39; and wherein said variable light chain comprises the amino acid chain set forth in SEQ ID NO: 40.

67. The pharmaceutical composition of claim 65, wherein the composition is formulated to dose by injection or infusion at 0.5 or more mg/kg, 1.0 or more mg/kg, 2.0 or more mg/kg, 4.0 or more mg/kg, or 10.0 or more mg/kg.

68. The pharmaceutical composition of claim 65, wherein the composition is formulated for administration twice a week, three time a week, weekly, bi-weekly, every three weeks, or monthly.

69. A pharmaceutical composition for use in treating, ameliorating, and/or preventing pancreatic cancer in a subject in need thereof, wherein the cancer is associated with expression of EPH Receptor A5, and the composition comprises the components Ab(antibody)-L(linker)-D(drug) as follows: 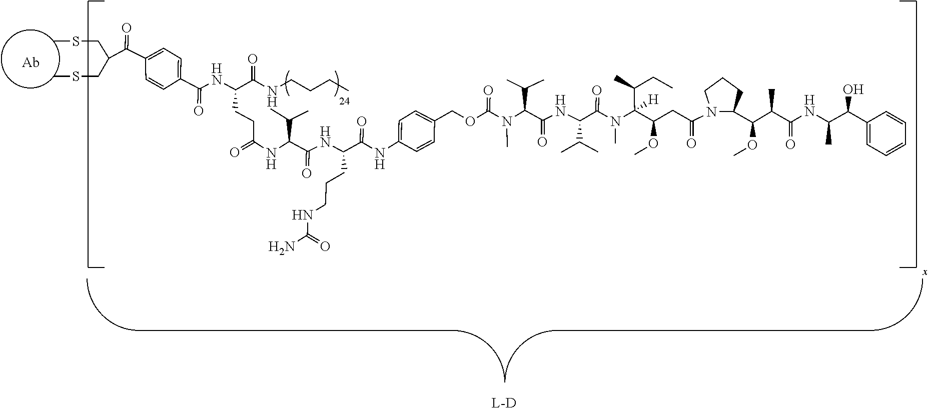
wherein the L-D portion of the composition has an x is from 1 to 8; and
wherein the Ab component is a human or humanized monoclonal antibody comprising:
(a) a first VH CDR comprising at least 90% sequence identity to the amino acid chain set forth in SEQ ID NO: 16;
(b) a second VH CDR comprising at least 90% sequence identity to the amino acid chain set forth in SEQ ID NO: 18;
(c) a third VH CDR comprising at least 90% sequence identity to the amino acid chain set forth in SEQ ID NO: 20;
(d) a first VL CDR comprising at least 90% sequence identity to the amino acid chain set forth in SEQ ID NO: 21;
(e) a second VL CDR comprising at least 90% sequence identity to the amino acid chain set forth in SEQ ID NO: 23; and
(f) a third VL CDR comprising at least 90% sequence identity to the amino acid chain set forth in SEQ ID NO: 24.

70. The pharmaceutical composition of claim 69, wherein said first VH CDR comprises the amino acid chain set forth in SEQ ID NO: 16; said second VH CDR comprises the amino acid chain set forth in SEQ ID NO: 18; said third VH CDR comprises the amino acid chain set forth in SEQ ID NO: 20; said first VL CDR comprises the amino acid chain set forth in SEQ ID NO: 21; said second VL CDR comprises the amino acid chain set forth in SEQ ID NO: 23; and said third VL CDR comprises the amino acid chain set forth in SEQ ID NO: 24.

71. The pharmaceutical composition of claim 69, wherein the composition is formulated to dose by injection or infusion at 0.5 or more mg/kg, 1.0 or more mg/kg, 2.0 or more mg/kg, 4.0 or more mg/kg, or 10.0 or more mg/kg.

72. The pharmaceutical composition of claim 69, wherein the composition is formulated for administration twice a week, three time a week, weekly, bi-weekly, every three weeks, or monthly.
